(19) United States
(12) Patent Application Publication
(10) Pub. No.: US 2010/0273207 A1
(43) Pub. Date: Oct. 28, 2010

(54) Methods for Diagnosis of Sepsis and Risk of Death

(76) Inventors: Raymond Langley, Albuquerque, NM (US); Stephen Kingsmore, St. Augustine, FL (US); Bo Chen, Durham, NC (US); Lawrence Carin, Durham, NC (US)

Correspondence Address: Ballard Spahr LLP, SUITE 1000, 999 PEACHTREE STREET, ATLANTA, GA, 30309-3915, US

(21) Appl. No.: 12/766,882

(22) Filed: Apr. 24, 2010

Related U.S. Application Data
(60) Provisional application No. 61/172,689, filed on Apr. 24, 2009.

(51) Int. Cl. C12Q 1/04

(52) U.S. Cl. 435/34

(57) ABSTRACT

The present invention is directed to methods for diagnosis of sepsis and high risk of sepsis death. Such methods are directed to subjects presenting to healthcare facilities with symptoms for sepsis. Clinico-metabolomic classifiers are tested and compared in the methods of the present invention.

### CROSS-REFERENCE TO RELATED APPLICATIONS

This application claims priority to U.S. Provisional Application No. 61/172,689 filed Apr. 24, 2009, which is hereby incorporated herein by reference in its entirety.

### BACKGROUND

Infection related complaints account for over 10 million emergency department visits in the United States annually. Sepsis, usually diagnosed by evidence of infection plus two or more SIRS criteria, causes an estimated 750,000 deaths per year and is the 10^{th} leading cause of death overall. The evaluation and management of patients with suspected sepsis is complicated by the lack of specific diagnostic criteria, heterogeneity of presentation and outcome. Early identification of patients likely to progress to death, who are candidates for aggressive treatment to prevent such death, is particularly difficult.

Current gold standards for prognostic assessment in sepsis include APACHE II (Acute Physiology and Chronic Health Evaluation), SOFA (Sepsis-related Organ Failure Assessment), and PRISM III (Pediatric Risk of Mortality) scores (Knaus et al., 1985; Vincent et al., 1996; Pollack et al., 1996). Additional potential treatments include admission to an intensive care unit, early goal directed therapy, activated protein C therapy, intensive glycemic control, hyperbaric or supplemental oxygen, or exogenous steroids (Otero et al., 2006; Russel 2008; Calzia et al., 2006; Muth et al., 2005; Annane 2005; Lin et al., 2005; Oter et al., 2005).

Biomarkers or reference characteristics are needed to stratify patients at presentation and identify patient subsets that need additional or more aggressive treatment. Additionally what is needed are methods for diagnosing sepsis and differentiating those with sepsis from those patients who do not have sepsis.

### SUMMARY OF THE INVENTION

The present invention comprises methods and kits for diagnosing sepsis in humans, methods for prognosis of a sepsis infection and outcomes, and methods for determining the sepsis status of a human who presents to a healthcare worker or facility as to whether the human does or does not have sepsis, and whether there is a high risk of death. Methods comprise measurement of the amounts of one or more clinico-metabolomic classifiers, which are identified clinical and metabolic changes in bodily fluids, such as plasma, of patients, for example, at time of presentation to a healthcare worker or facility, that distinguish sepsis from other disorders with similar presentation (NIS—non-infected SIRS-positive) (SIRS—systemic inflammatory response) and that differentiate sepsis patients that are likely to have uncomplicated courses from those patients that are likely to have complications, including death. An aspect of the methods comprises novel therapeutic targets for individualized intervention. Disclosed herein are methods and compositions of diagnosing sepsis in a human subject. Also disclosed are methods and compositions of identifying subjects at high risk of sepsis death.

### BRIEF DESCRIPTION OF THE DRAWINGS

The accompanying drawings, which are incorporated in and constitute a part of this specification, illustrate embodiments of the invention, and together with the description, serve to explain the principles of the methods and systems:

FIG. 1A-B shows Z-score scatter plots for biochemicals observed in human plasma obtained from subjects with SIRS (mean=0.71), which is used as the control, and from subjects with UCS (mean=0.78), subjects with severe sepsis (mean=0.97), subjects with septic shock (mean=0.78), and subjects with sepsis that died (mean=0.94); FIG. 1B shows the Z-score scatter plots for sepsis patients only, using the UCS group as the control (UCS (mean=0.71), severe sepsis (mean=1.26), septic shock (mean=0.91), and septic death (mean=1.33);

FIG. 2 shows variance decomposition with Pearson product-moment correlation for sepsis groups (SIRS+, UCS, severe sepsis, septic shock, and septic death) as well as chronic kidney disease and hemodialsyis (CKD(HD)), liver disease, and immunosuppressant therapy, and demonstrates that CDK(HD), liver disease and immunosuppreseant therapy are substantive, non-hypothesis components of variance. In FIG. 2, CKD(HD)=estimated glomerular filtration rate (eGFR) per chronic kidney disease criteria, and hemodialysis;

FIG. 3A-B shows the normalized energies of sepsis group membership (SIRS+Outcomes), renal category (CKD(HD)) and other clinical parameters in log-transformed plasma metabolites at t₀ (A) and t₂₄ (B). In FIG. 3, sepsis group membership (SIRS+Outcomes) was defined as non-infected SIRS-positive, sepsis survival, and sepsis death, and renal function was defined as (i) eGFR>74 mL/min=0; (ii) eGFR>32-74 mL/min=1; or (iii) eGFR<31 mL/min=2, and hemodialysis=3. Clinical parameters were fit to a normal distribution with mean of zero and standard deviation of one. Normalized energy of sepsis group membership (SIRS+Outcomes) increased from 0.06 at t₀ to 0.14 at t₂₄;

FIG. 4A-D shows variance decomposition with Pearson correlation of sepsis diagnosis (non-infected SIRS positive vs. sepsis survivor groups) at t₀ (A) and t₂₄ (B) and shows volcano plots illustrating significant metabolite differences between groups (i.e., the points above the line) by ANOVA with non-hypothesis components of variance as fixed effects at t₀ (FDR 10%) (C) and t₂₄ (FDR 5%) (D);

FIG. 5A-D shows plasma metabolite changes in sepsis outcomes (survival or death) in the discovery cohort at t₀ (A) and t₂₄ (B), and in the replication cohort at t₀ (C) and t₂₄ (D). In FIG. 5A-5D, the left panel shows variance decomposition with Pearson correlation of known parameters. In FIG. 5A-5D, the right panel shows volcano plots illustrating significant metabolite differences (i.e., the points above the line) by ANOVA with non-hypothesis variance parameters as fixed effects. At t₀ and t₂₄, FDR=5%. At t₀, replication=25% at t₂₄, replication=15%;

FIG. 6A-D shows plasma metabolite levels for 16 metabolites in sepsis at t₀ (A), t₂₄ (B) and in replication patients at t₀ (C) and t₂₄ (D). Y-axis displays average, scaled, plasma metabolite concentrations. Error bars are SEM. In (A) and (B), from left to right for each metabolite, the three columns respectively represent controls (non-infected, SIRS positive), sepsis survivors, and sepsis deaths. In (C) and (D), from left to right for each metabolite, the columns respectively represent sepsis survivors and sepsis death. Asterisks indicate significant differences from sepsis survivors by weighted ANOVA with 5% FDR in (A) and (B), 25% FDR in (C), or 15% FDR in (D);

FIG. 7A-D shows plasma metabolite levels for 16 metabolites in sepsis at t₀ (A), t₂₄ (B) and in replication patients at t₀ (C) and t₂₄ (D). Y-axis displays average, scaled, plasma metabolite concentrations. Error bars are SEM. In (A) and (B), from left to right for each metabolite, the three columns respectively represent controls (non-infected, SIRS positive), sepsis survivors, and sepsis deaths. In (C) and (D), from left to right for each metabolite, the columns respectively represent sepsis survivors and sepsis death. Asterisks indicate significant differences from sepsis survivors by weighted ANOVA with 5% FDR in (A) and (B), 25% FDR in (C), or 15% FDR in (D);

FIG. 8A-D shows plasma metabolite levels for 16 metabolites in sepsis at t₀ (A), t₂₄ (B) and in replication patients at t₀ (C) and t₂₄ (D). Y-axis displays average, scaled, plasma metabolite concentrations. Error bars are SEM. In (A) and (B), from left to right for each metabolite, the three columns respectively represent controls (non-infected, SIRS positive), sepsis survivors, and sepsis deaths. In (C) and (D), from left to right for each metabolite, the columns respectively represent sepsis survivors and sepsis death. Asterisks indicate significant differences from sepsis survivors by weighted ANOVA with 5% FDR in (A) and (B), 25% FDR in (C), or 15% FDR in (D);

FIG. 9A-D shows plasma metabolite levels for 17 metabolites in sepsis at t₀ (A), t₂₄ (B) and in replication patients at t₀ (C) and t₂₄ (D). Y-axis displays average, scaled, plasma metabolite concentrations. Error bars are SEM. In (A) and (B), from left to right for each metabolite, the three columns respectively represent controls (non-infected, SIRS positive), sepsis survivors, and sepsis deaths. In (C) and (D), from left to right for each metabolite, the columns respectively represent sepsis survivors and sepsis death. Asterisks indicate significant differences from sepsis survivors by weighted ANOVA with 5% FDR in (A) and (B), 25% FDR in (C), or 15% FDR in (D);

FIG. 10A-D shows principal components of plasma metabolomic variance with Pearson product-moment correlation of sepsis survivor groups at t₀ (A) and volcano plots of FDR-corrected (5%) ANOVAs with non-hypothesis components of variance as fixed effects (B, C, and D). In (B)-(D), significantly altered metabolite levels are indicated by dots above the line; however, the variation between these groups was 1.7%;

FIG. 11A-D shows principal components of variance decomposition with Pearson product-moment correlation for etiologic agents and clinical parameters at t₀ (A) and volcano plots of FDR-corrected (5%) ANOVAs (with non-hypothesis components of variance as fixed effects) (B, C, and D). The volcano plots indicate no significant differences between host metabolic response to bacteremia with E. coli (n=16) and S. pneumoniae (n=31) (B), or E. coli and S. aureus (n=27) (C), or S. pneumoniae and S. aureus (D). In (B)-(D), the variation explicable by these groups was 0.2%;

FIG. 12A-C shows Venn diagrams of significant differences (weighted ANOVA, 5% FDR) in plasma metabolite levels between non-infected patients (with SIRS) and sepsis survivors at t₀ and t₂₄ (A), concordance of direction of change of significantly altered metabolites (B), and concordance of direction of change of metabolites exhibiting significant differences at one of the time points (C);

FIG. 13A-E shows Venn diagrams of significant differences in plasma metabolite levels between sepsis survivors and deaths at t₀ and t₂₄ in the discovery and replication (Rt₀ and Rt₂₄) cohorts (A), concordance of direction of change of significantly different metabolites (B) and (D), and concordance of direction of change of metabolites with significant differences at one of the time points (C) and (E). Significant differences reflect weighted ANOVAs with 5% FDR (t₀ and t₂₄ in the discovery set), 25% FDR (to in the replication set) or 15% FDR (t₂₄ in the replication set); and

FIG. 14 shows the results of measuring nearly 550 biochemicals in venous plasma of subjects with non-infected SIRS positive (SIRS), subjects that survived sepsis (Sepsis Survivors or SS), and subjects with sepsis that died (Sepsis Deaths or SD). The biochemicals were measured at t₀ and t₂₄ using label-free, combined liquid and gas chromatography and MS.

### DETAILED DESCRIPTION

The present invention comprises methods and kits for diagnosing sepsis in humans, methods for prognosis of a sepsis infection and outcomes, and methods for determining the sepsis status of a human who presents to a healthcare worker or facility as to whether the human does or does not have sepsis, and whether the human with sepsis will live or if a high risk of death from sepsis exists. Methods comprise measurement of the amounts of one or more clinico-metabolomic classifiers, which are identified clinical and metabolic changes in bodily fluids, such as plasma, of patients, for example, at the time of presentation to a healthcare worker or facility, that distinguish sepsis from other disorders with similar presentation, NIS, (non-infected patients SIRS-positive), or SIRS, (systemic inflammatory response), and that differentiate sepsis patients that are likely to have uncomplicated courses from those patients that are likely to have complications, including death. As used herein, NIS and SIRS refer to the same or similar condition, and are used interchangeably.

An aspect of the present invention comprises, in a human, taking a bodily fluid sample from the human, and using an instrument to measure individual clinico-metabolomic classifiers, or measuring individual clinico-metabolomic classifiers directly without the need for taking a bodily sample from the human, and determining the level or amount of the clinico-metabolomic classifier (obtaining a value for the clinico-metabolomic classifier) or a change in the level or amount of the clinico-metabolomic classifier. The value or change in the level or amount of one or more clinico-metabolomic classifiers is compared to values or changes in the level or amount of the clinico-metabolomic classifier in a group of comparative subjects, such as subjects who had sepsis but did not die, or subjects who had sepsis and died, or in subjects who had SIRS or NIS, conditions that are not sepsis but have confusingly similar symptoms.

Particular changes in the amount or level of one or more clinico-metabolomic classifiers provide identification of the status of the subject as to its sepsis status, and these changes form a reference characteristic. For example, an increase in one or more particular clinico-metabolomic classifiers may be a reference characteristic for a condition such as sepsis, or high risk of death from sepsis, or lack of sepsis. The combination of the values or the changes in a particular set of clinico-metabolomic classifier may form a reference characteristic, or a pattern of response by the body, for a sepsis status, including sepsis, no sepsis or sepsis that results in death. In the disclosure, particular reference characteristics are noted, though all changes or clinico-metabolomic classifiers listed which may be reference characteristics may not be so identified. Exemplary reference characteristics are disclosed in the Examples herein.

A method of the present invention for diagnosis of a sepsis condition or the sepsis status, including diagnosing sepsis, high risk of death from sepsis, or the presence of SIRS/NIS or a condition that is not sepsis, may be determined in a human with symptoms at initial presentation to a healthcare worker or facility, or within 24 hours, 36 hours, 48 hours, 3 days, 4 days, or more days, including 25, 26, 27, 28, 29 or 30 days. Methods of determining the sepsis status of a human may be used one time or more than one time, for example, at initial presentation, and during the course of treatment to provide prognosis and monitoring of the sepsis status of the human.

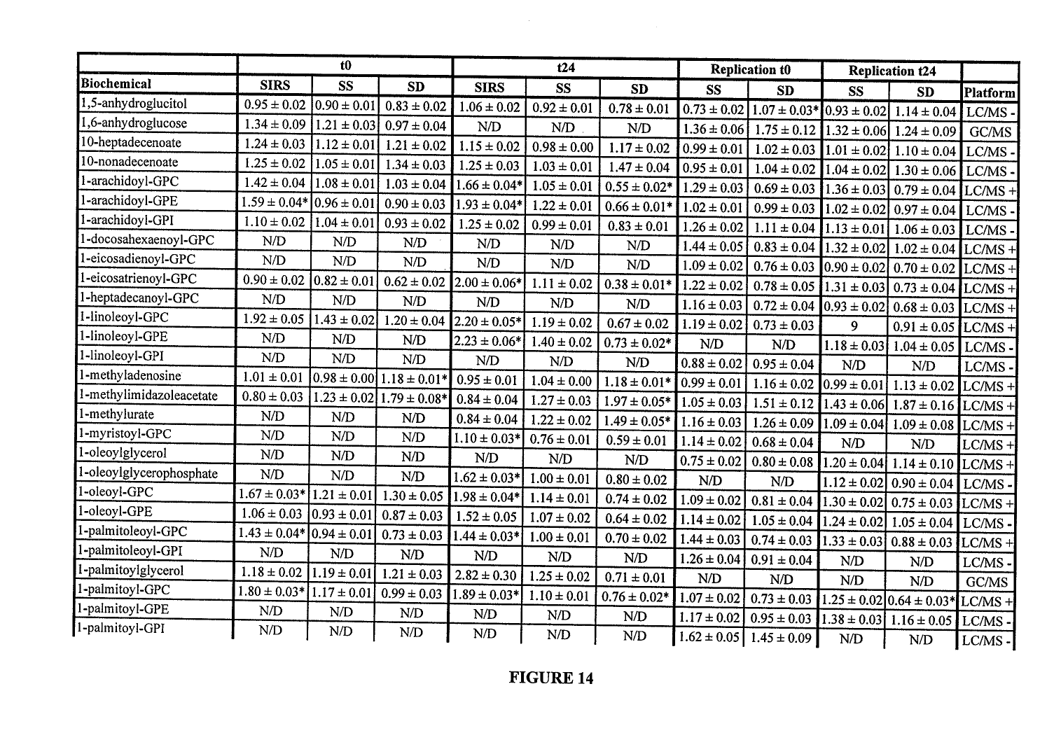

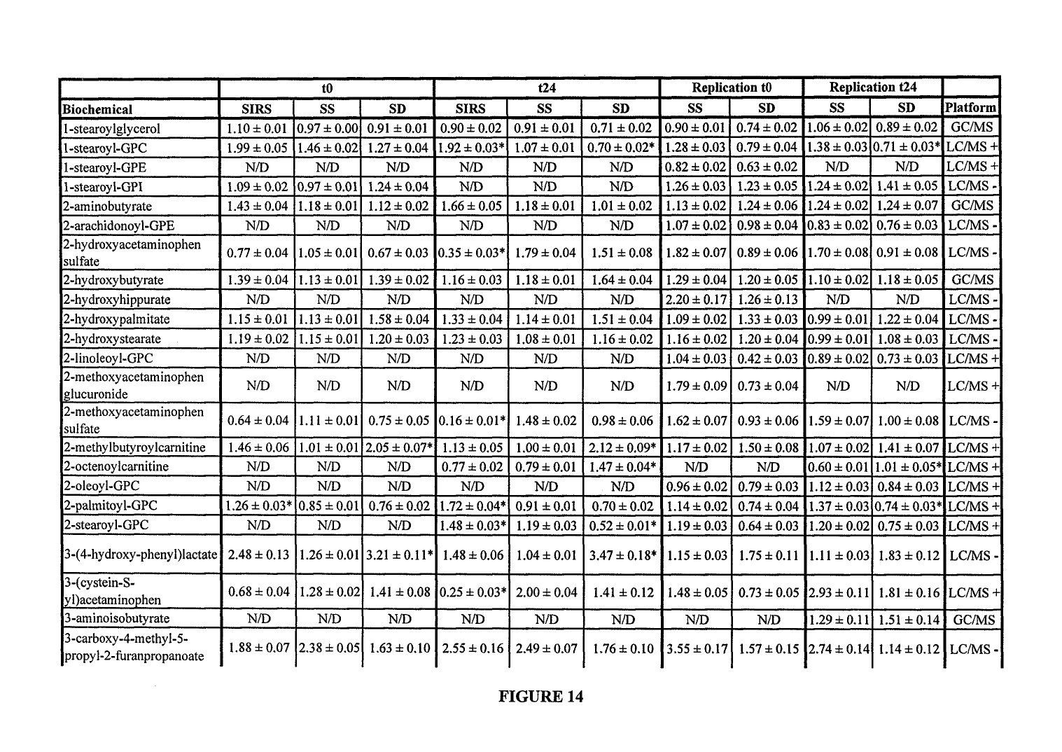

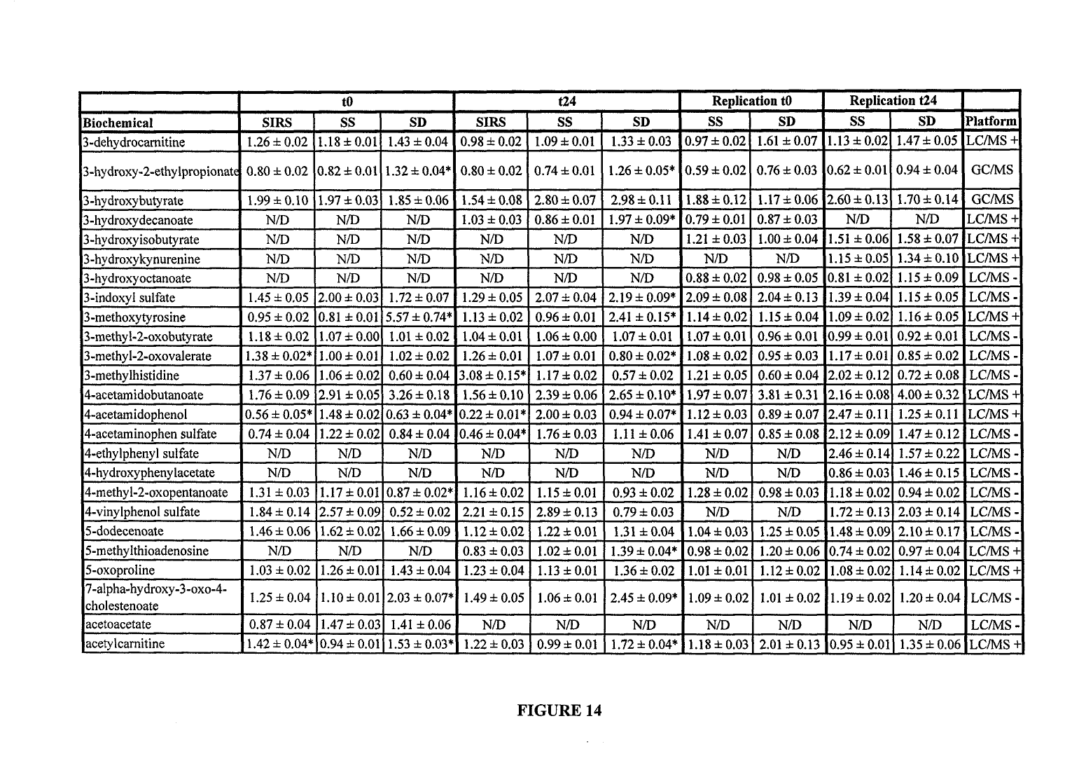

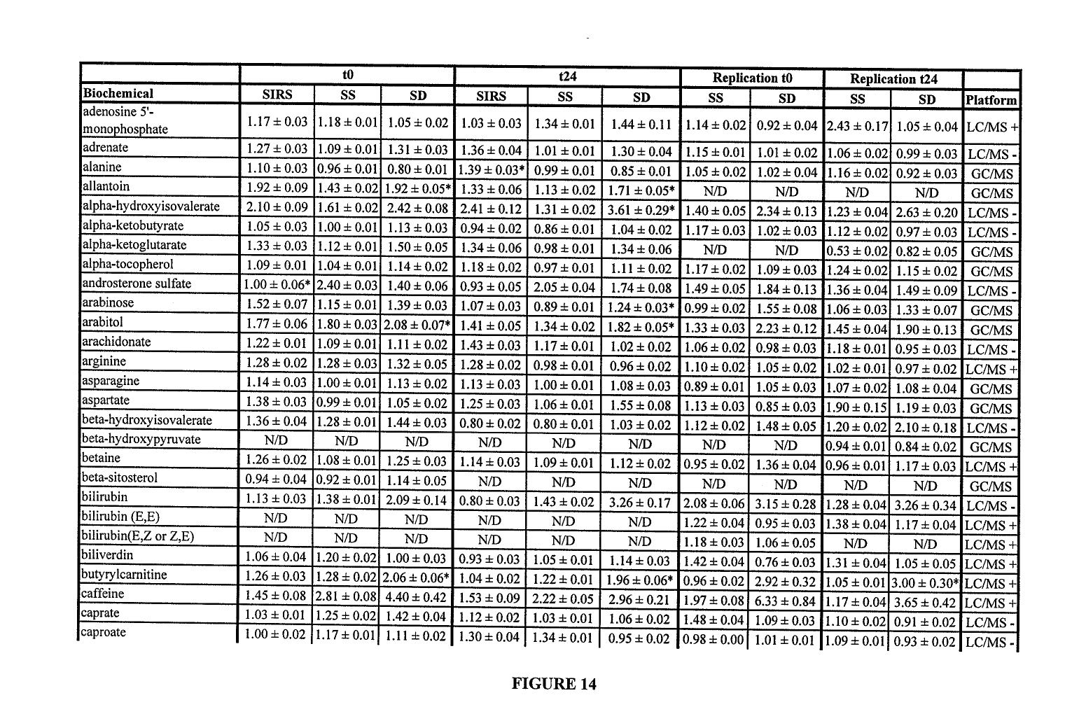

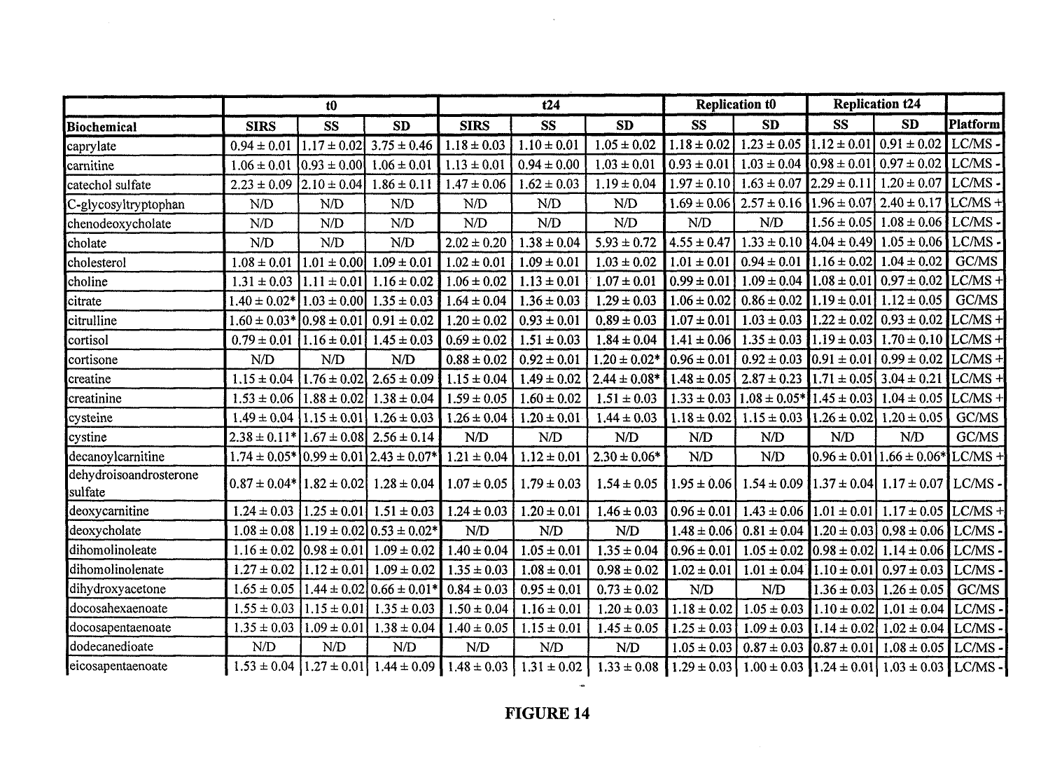

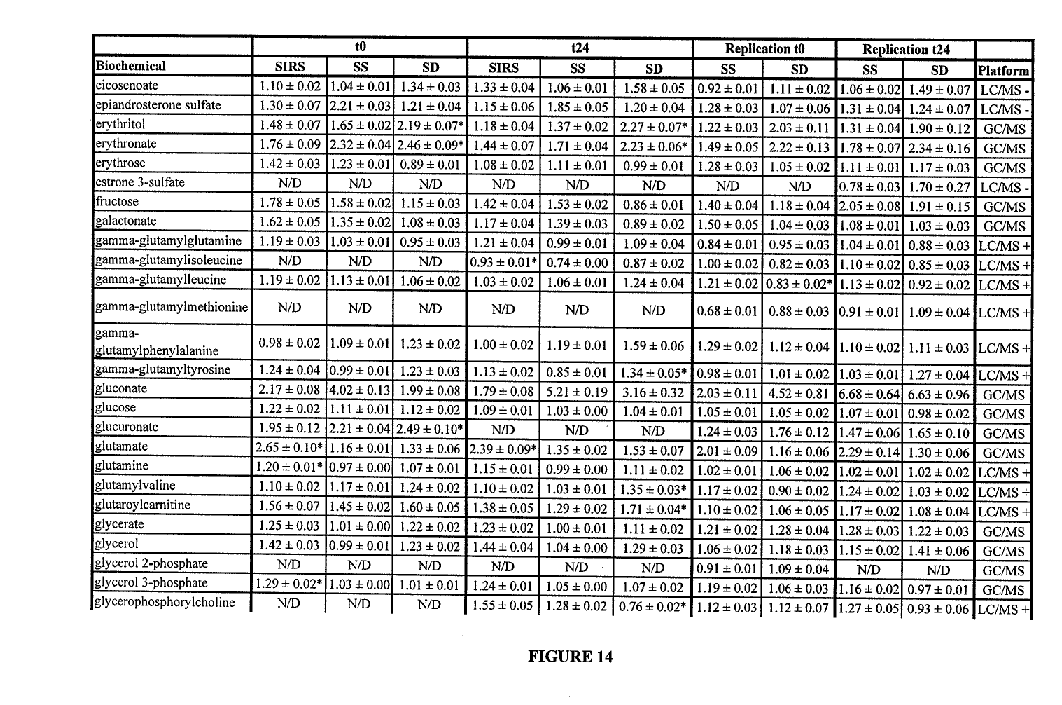

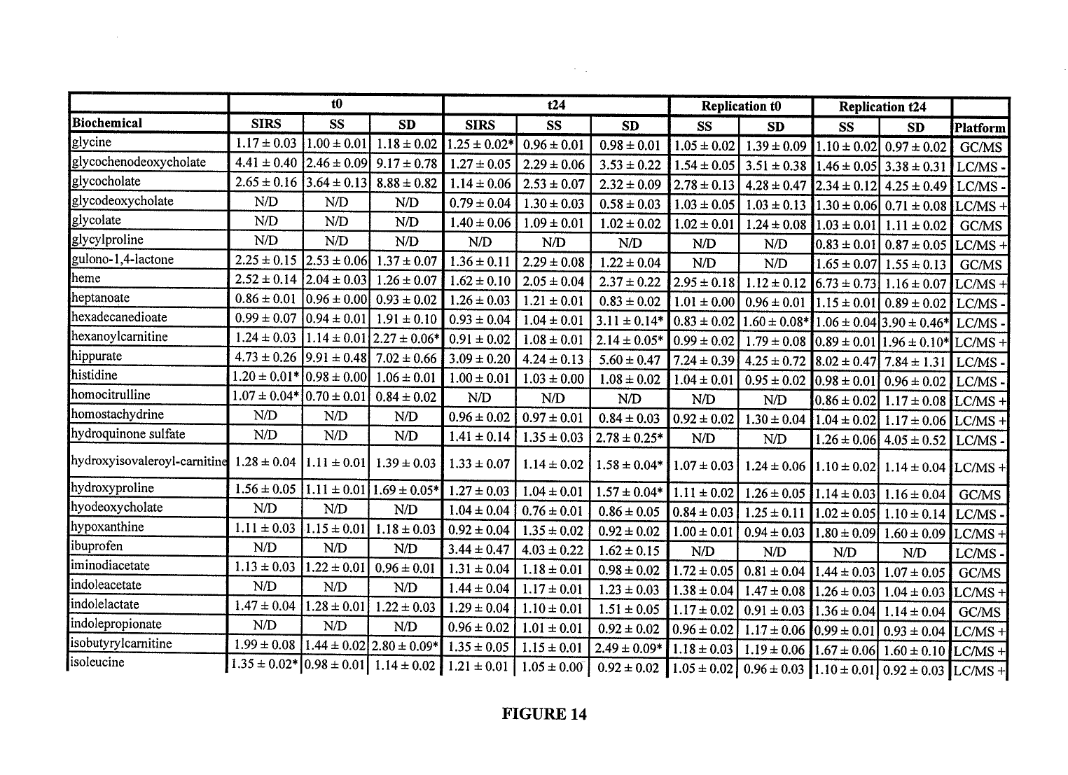

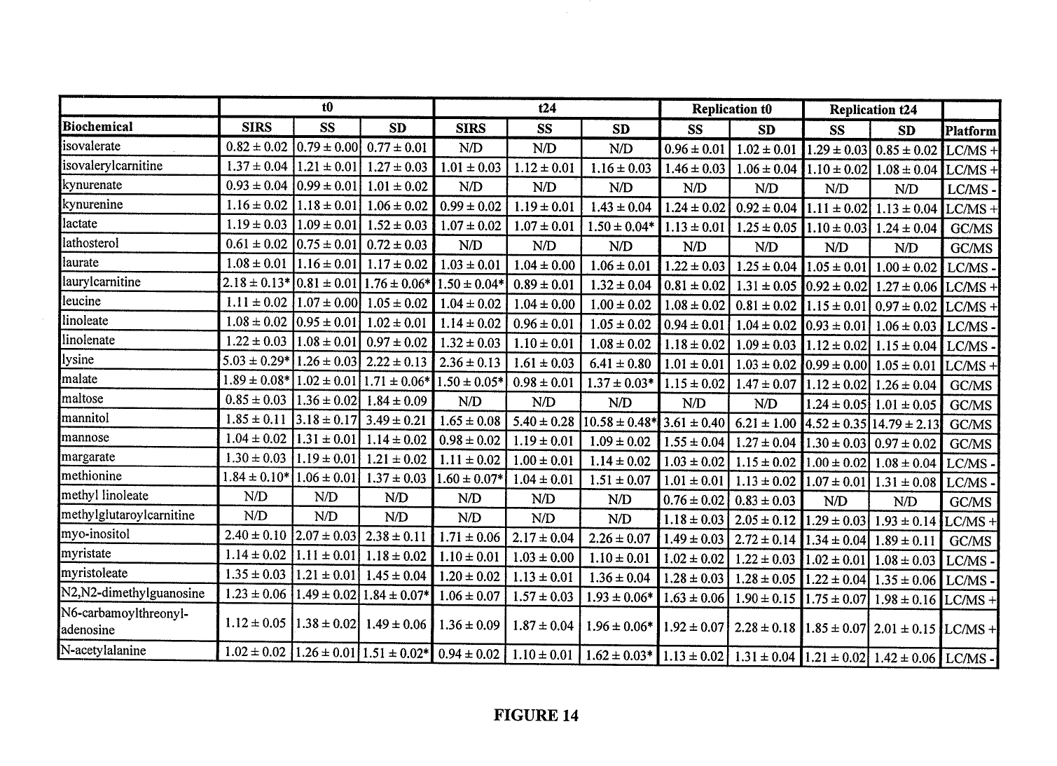

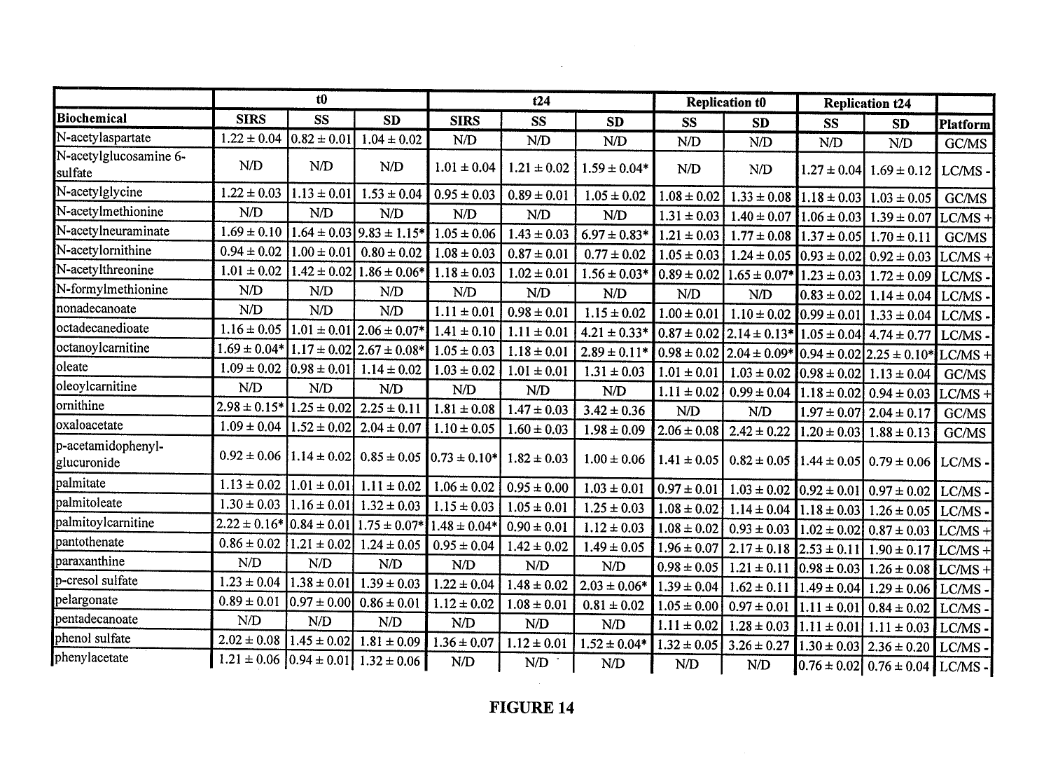

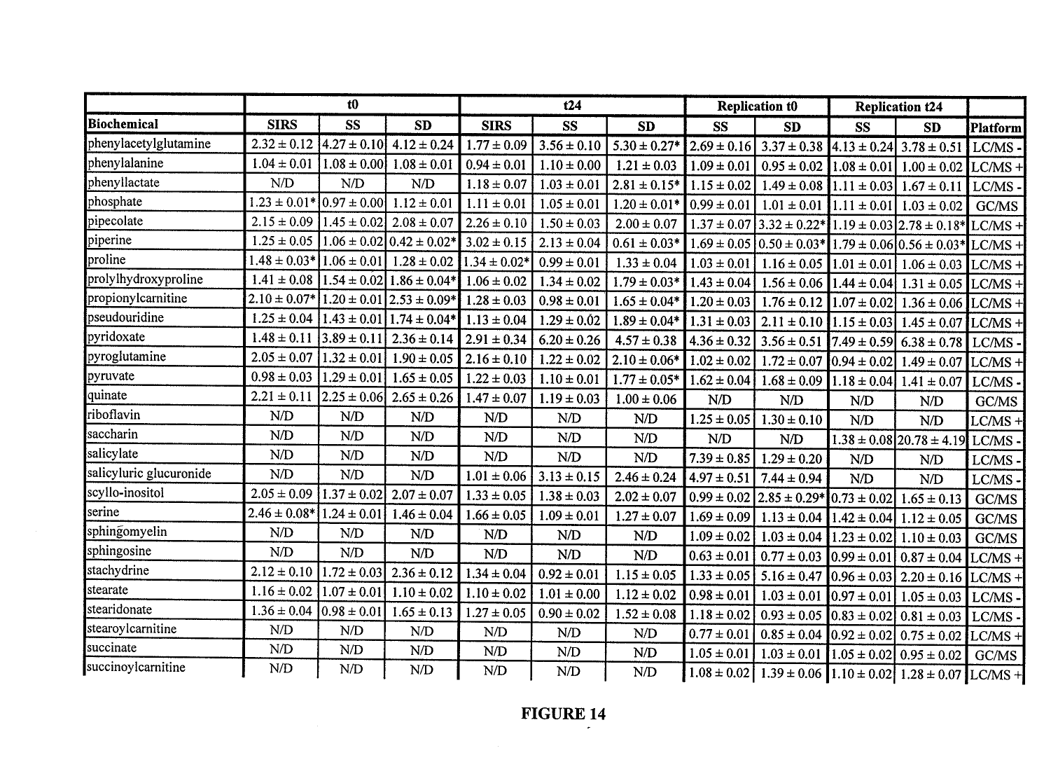

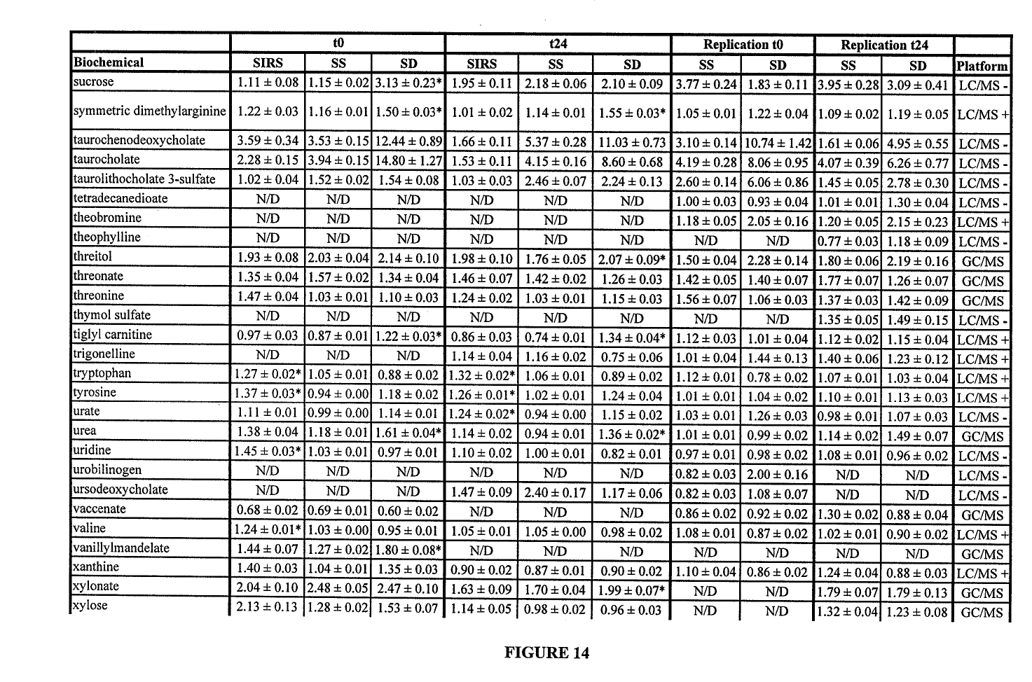

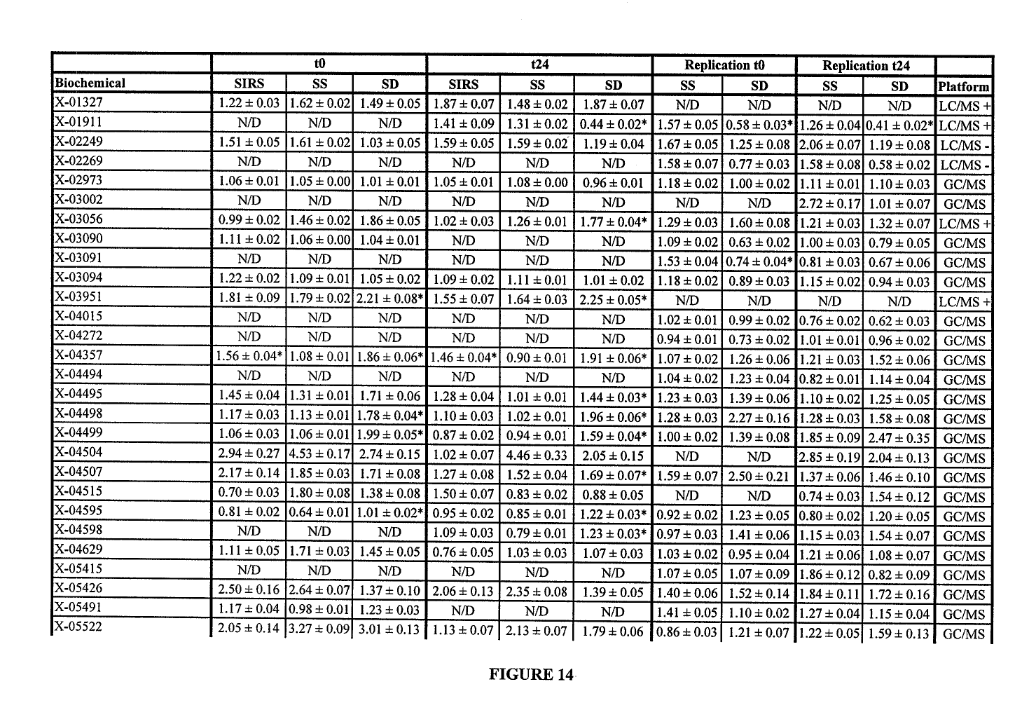

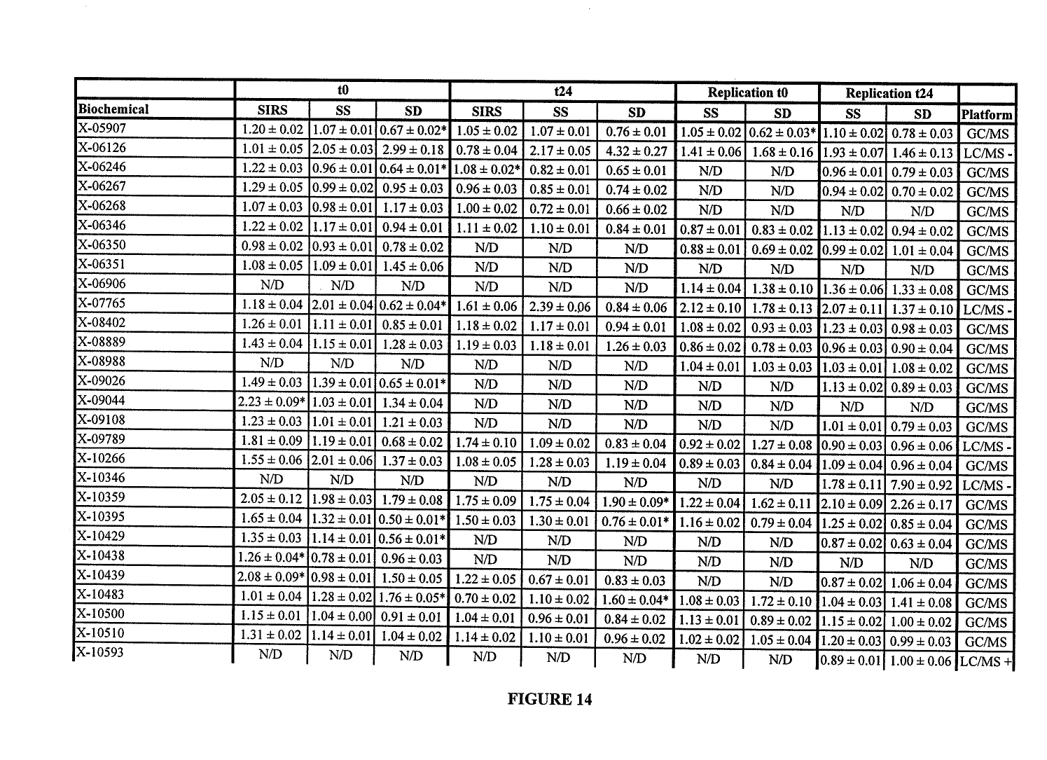

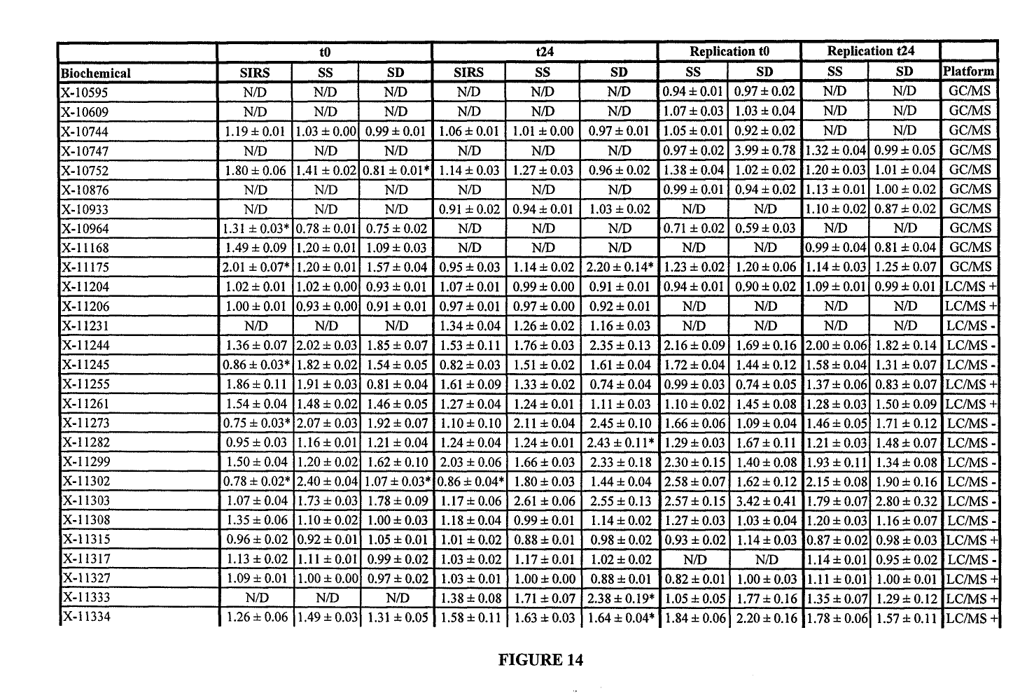

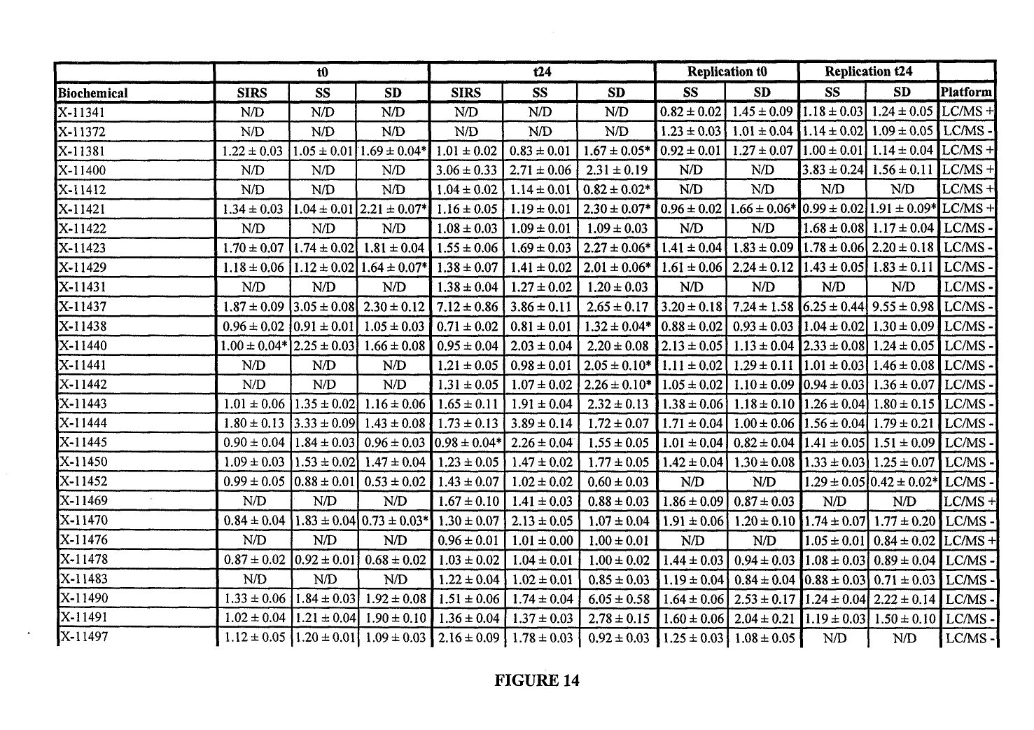

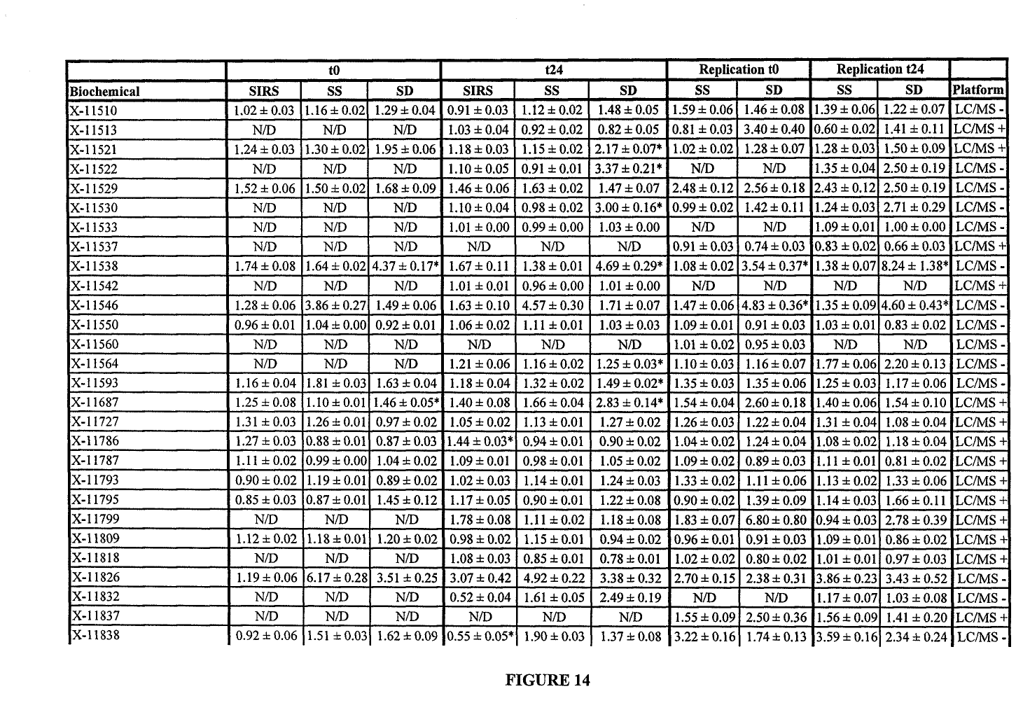

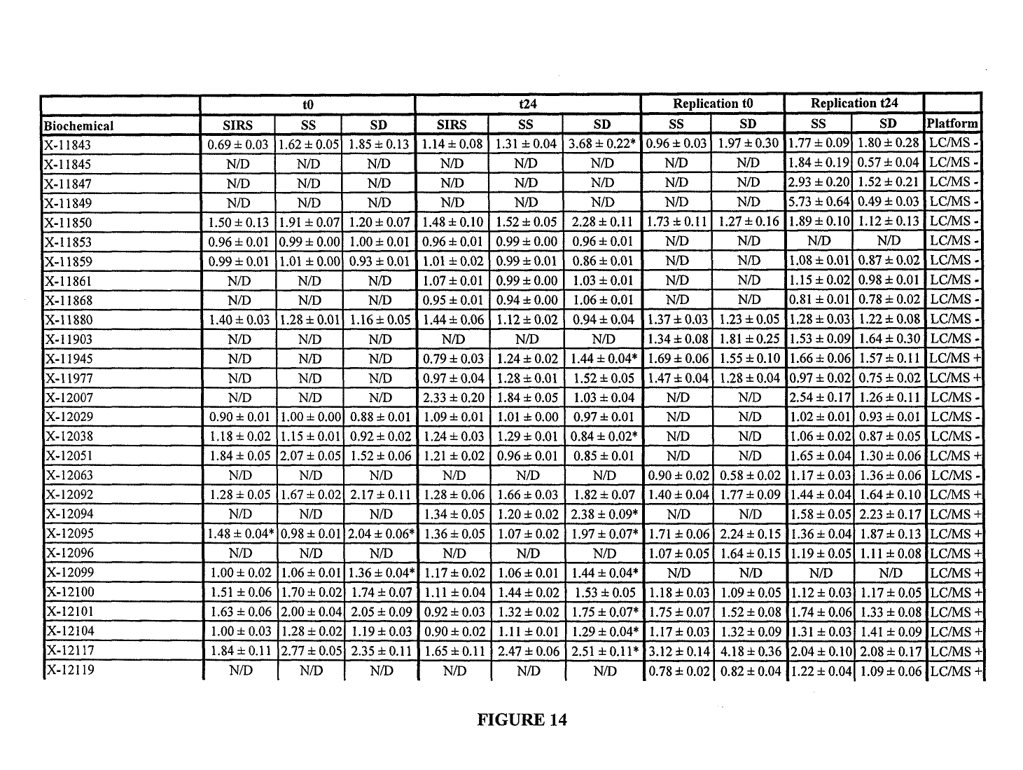

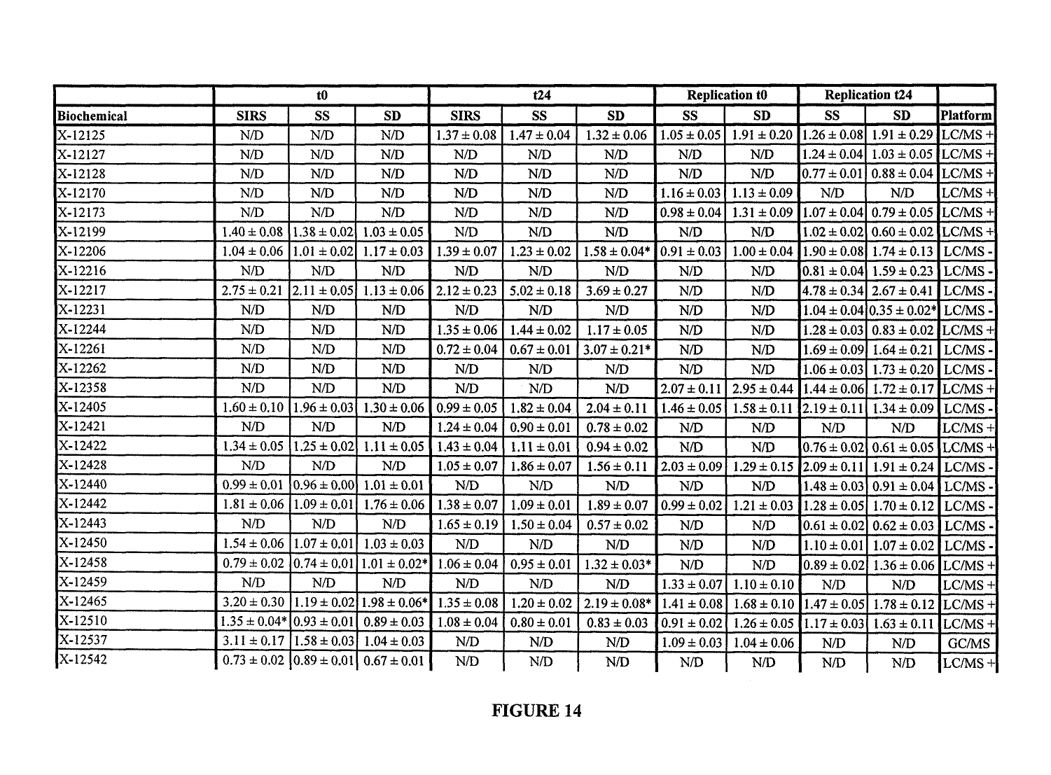

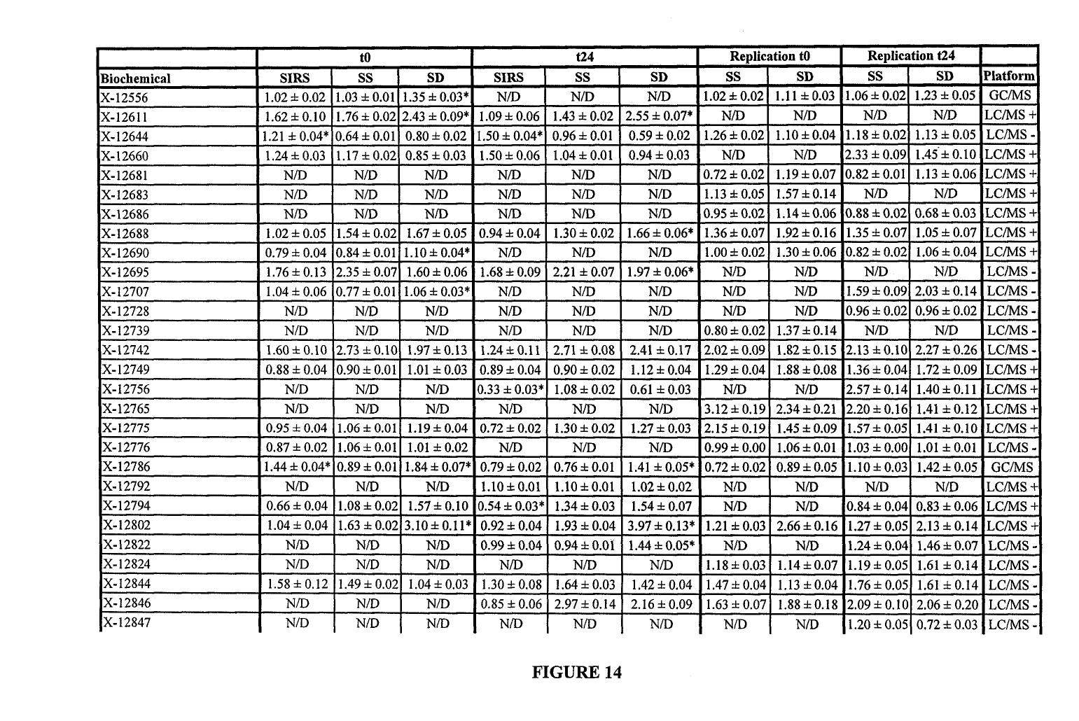

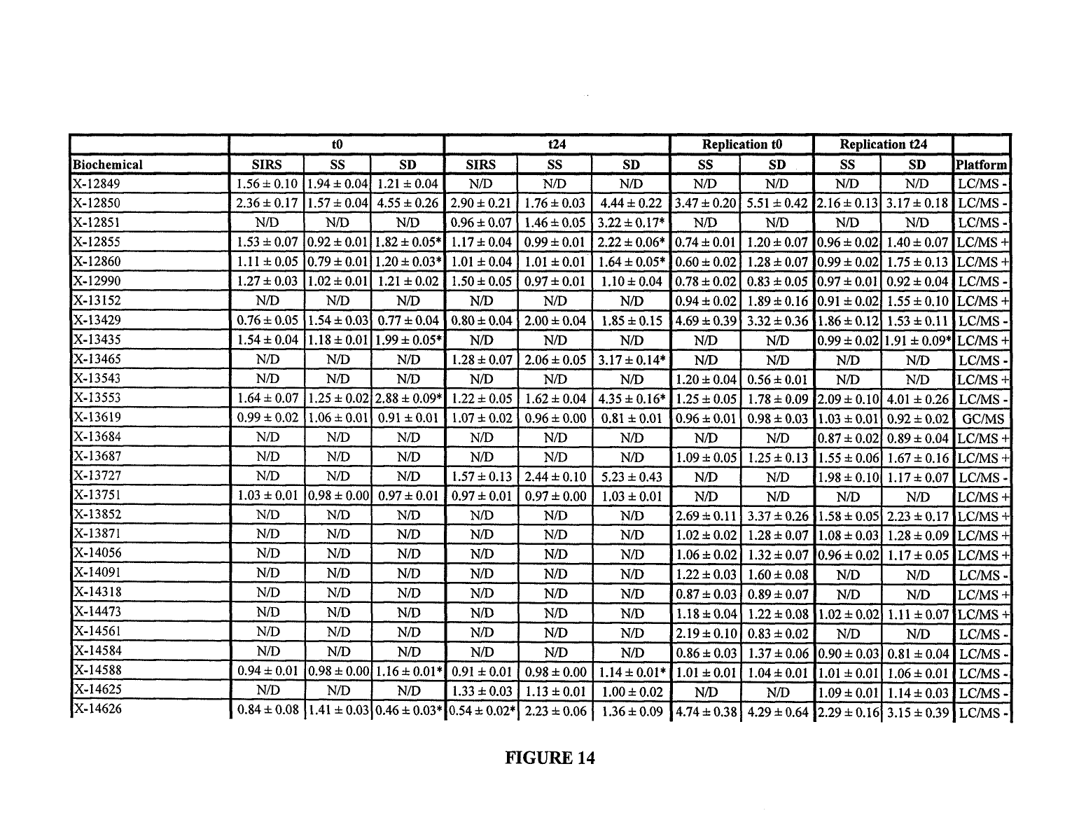

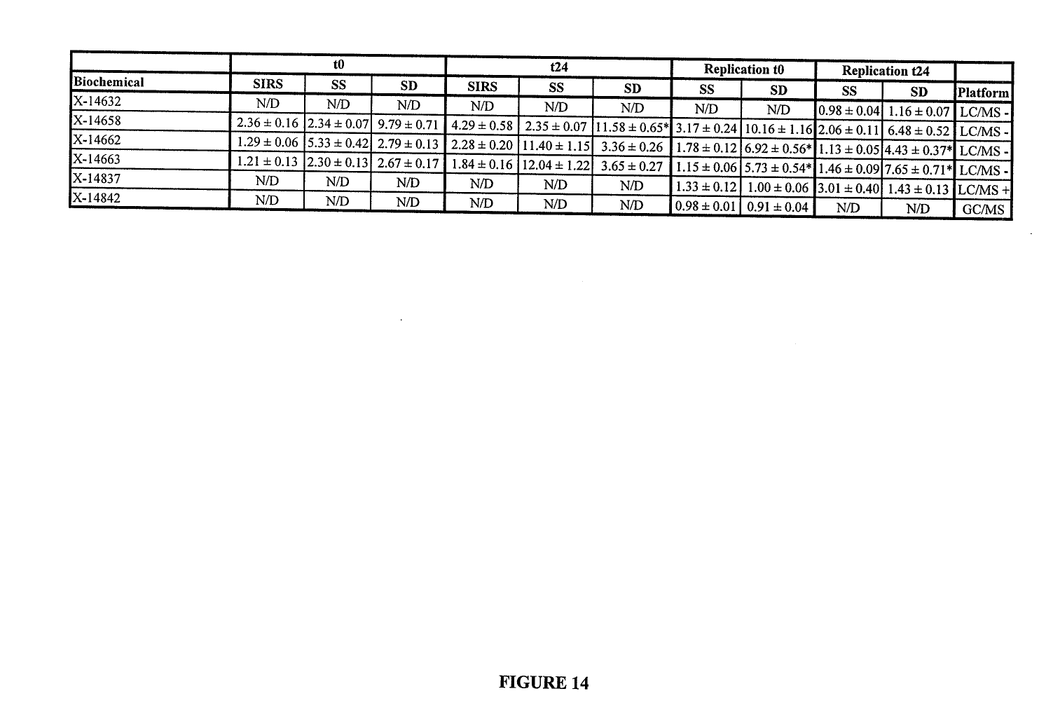

As used herein, clinico-metabolomic classifier means one of an analyte, unannotated analyte or clinical evaluators utilized by the disclosed methods and kits. For example, a method of the present invention comprises measuring one or more clinico-metabolomic classifiers comprising amino acids, peptides, carbohydrates, lipids, xenobiotics, cofactors, vitamins, and nucleotides. FIG. 14 lists exemplary nearly 550 analytes that can be useful in the present invention. Analytes can be measured in the subject's bodily fluid, such as blood, plasma, and serum. Analytes include, but are not limited to, citrulline, serine, creatine, lysine, glutamate, indolepropionate, laurylcarnitine, fructose, erythrose, glycerol-3-phosphate, 10-undecenoate, androsterone-sulfate, epiandrosterone-sulfate, dihydroepiandrosterone-sulfate (DHEAS), unannotated metabolite X-09789, X-11302, temperature, mean arterial pressure, billirubin, threonine, glycine, alanine, laurylcarnitine, acetylcarnitine, decanoylcarnitine, lactate, pyruvate, maltose, 1-palmitolcoylglycerophosphocholine (1-palmitoleoyl-GPC, C16:1), DHEAS, and unannotated metabolite X-11302.

Clinico-metabolomic classifiers include unannotated analytes designated in the text by the nomenclature “X-nnnnn”, where “n” is an integer. These unannotated analytes are biochemicals that can be unambiguously identified by their retention time (RT) and mass. For all liquid chromatography (LC) compounds, the masses represent the neutral mass of the quant ion, which may be the compound or an adduct of the compound. Because gas chromatography (GC) compounds are derivatized, the mass represents the mass of the derivatized compound, which was derivatized via silylation with trimethylsilane. The following unannotated metabolites are comprised in the disclosed methods and kits.

TABLE 1
UNANNOTATED ANALYTES

| | | | Retention | Quant | | |
|---|---|---|---|---|---|---|
| Analyte | ID | Platform | Time | Ion Mass | Polarity | Tentative Details |
| X-09789 | 32753 | LC/MS− | 2.62 | 153.1 | −i | probable empirical formula |
| | | | | | | C7H6O4; possible cmpd: |
| | | | | | | cyclopenta-1,3-diene-1,2- |
| | | | | | | dicarboxylic acid |
| X-11302 | 32619 | LC/MS− | 5.01 | 397.3 | −i | pregn steroid monosulfate |
| X-10395 | 25459 | GC/MS | 9.94 | 156.0 | +L | |
| X-11538 | 32855 | LC/MS− | 4.93 | 312.3 | −i | |
| X-11908 | 33253 | LC/MS− | 5.27 | 429.4 | −i | retired for 7alpha-hydroxy-3-oxo- |
| | | | | | | 4-cholestenoic acid |
| X-11444 | 32761 | LC/MS− | 3.99 | 541.2 | −i | acc mass from MTRX plasma, JL, |
| | | | | | | Jun. 25, 2009 |
| X-02249 | 32587 | LC/MS− | 4.03 | 267.2 | −i | probable empirical formula |
| | | | | | | C14H20O5; possible cmpd: 3- |
| | | | | | | carboxy-4-methyl-5-pentyl-2- |
| | | | | | | furanpropionic acid |
| X-03056 | 32709 | LC/MS+ | 2.21 | 185.2 | +i | |
| X-06126 | 32557 | LC/MS− | 2.69 | 203.1 | −i | |
| X-07765 | 32560 | LC/MS− | 3.71 | 245.1 | −i | |
| X-11245 | 32562 | LC/MS− | 3.91 | 238.3 | −i | pregnen-diol disulfate |
| X-11255 | 32572 | LC/MS+ | 2.38 | 247.1 | +i | retired for betaine tryptophan p |
| X-11261 | 32578 | LC/MS+ | 3.69 | 286.2 | +i | empirical formula C15H27NO4; |
| | | | | | | possible cmpd: 2-octenoyl |
| | | | | | | carnitine |
| X-11301 | 32618 | LC/MS+ | 0.83 | 144.2 | +i | replaced with X-11512 |
| X-11302 | | LC/MS− | 5.01 | 398.3 | | sulfated compound |
| X-11381 | 32698 | LC/MS+ | 1.11 | 186.2 | +i | acc mass from MTRX plasma, |
| | | | | | | probable empirical formula |
| | | | | | | C9H15NO3 |
| X-11421 | 32738 | LC/MS+ | 4.54 | 314.2 | +i | MS/MS suggests a carnitine |
| | | | | | | probably 10:1 |
| X-11423 | 32740 | LC/MS− | 1.05 | 260.1 | −i | |
| X-11444 | | LC/MS− | 1.91 | 401.3 | | |
| X-11445 | 32762 | LC/MS− | 4.01 | 239.3 | −i | 5alpha-pregnan-3beta,20alpha-diol |
| | | | | | | disulfate |
| X-11450 | 32767 | LC/MS− | 4.11 | 224.2 | −i | Disulfated steroid |
| X-11478 | 32795 | LC/MS− | 4.30 | 165.2 | −i | |
| X-11538 | | LC/MS− | 4.93 | 312.2 | | |
| X-11546 | 32863 | LC/MS− | 5.02 | 448.4 | −i | |
| X-11793 | 33138 | LC/MS+ | 3.57 | 601.1 | +i | |
| X-11809 | 33154 | LC/MS+ | 5.23 | 381.5 | +i | |
| X-11826 | 33171 | LC/MS− | 1.48 | 194.1 | −i | Retired for p-hydroxyhippuric acid |
| X-11838 | 33183 | LC/MS− | 2.30 | 276.0 | −i | |
| X-11843 | 33188 | LC/MS− | 2.69 | 230.1 | −i | |
| X-11908 | | LC/MS− | 5.27 | 430.3 | | possible form of cholestonic acid |
| X-12029 | 33380 | LC/MS− | 0.68 | 329.1 | −i | |
| X-12051 | 33403 | LC/MS+ | 5.83 | 456.4 | +i | empirical formula C26H46O5; |
| | | | | | | possible cmpd: Ranol; Also |
| | | | | | | possible 27-nor-pentol |
| | | | | | | HMDB02126 |
| X-12092 | 33507 | LC/MS+ | 1.13 | 144.2 | +i | |
| X-12095 | 33510 | LC/MS+ | 1.60 | 153.1 | +i | |
| X-12100 | 33515 | LC/MS+ | 1.76 | 221.1 | +i | cannot be the 5-form - maybe the |
| | | | | | | 4 or 7-form empirical formula |
| | | | | | | C11H12N2O3 |
| X-12117 | 33532 | LC/MS+ | 1.44 | 204.2 | +i | Accurate mass 203.12698; |
| | | | | | | empirical formula C8H17N3O3; |
| | | | | | | Possible ala-orn or gly-lys |
| X-12206 | 33627 | LC/MS− | 0.64 | 255.1 | −i | |
| X-12217 | 33638 | LC/MS− | 2.32 | 203.1 | −i | possible sulfate |
| X-12405 | 33833 | LC/MS− | 2.55 | 212.1 | −i | |
| X-12442 | 33884 | LC/MS− | 5.28 | 223.4 | −i | |
| X-12465 | 33910 | LC/MS+ | 1.41 | 248.2 | +i | identical to X-12855 but elutes |
| | | | | | | approx 70 RI units later; probably |
| | | | | | | 3-hydroxybutyrylcarnitine or |
| | | | | | | similar; A carnitine - AME |
| X-12644 | 34244 | LC/MS− | 5.74 | 524.3 | −i | elutes approx 50 units after X- |
| | | | | | | 12645; isomeric with X12645, but |
| | | | | | | reasonably resolved |
| X-12688 | 34297 | LC/MS+ | 1.20 | 203.2 | +i | |
| X-12695 | 34305 | LC/MS− | 0.72 | 144.1 | −i | |
| X-12742 | 34352 | LC/MS− | 2.56 | 241.2 | −i | empirical formula C11H18N2O4 |
| | | | | | | AME; Possible glutamine |
| | | | | | | conjugate. AME |
| X-12775 | 34452 | LC/MS+ | 1.80 | 164.1 | +i | |
| X-12794 | 34477 | LC/MS+ | 0.87 | 264.2 | +i | |
| X-12802 | 34485 | LC/MS+ | 2.72 | 318.2 | +i | |
| X-12855 | 34539 | LC/MS+ | 1.36 | 248.2 | +i | looks identical to X-12465 but |
| | | | | | | elutes approx 70 Units earlier; |
| | | | | | | probably either 3- |
| | | | | | | hydroxybutyrlcarnitine or similar |
| X-12860 | 34544 | LC/MS+ | 2.25 | 276.2 | +i | X-12860 and X-13743 have |
| | | | | | | similar ms/ms; X-13743 elutes |
| | | | | | | approx 170 units after X-12860; |
| | | | | | | appears to be a carnitine |
| X-13553 | 35331 | LC/MS− | 1.42 | 263.0 | −i | |
| X-14588 | 36515 | LC/MS− | 0.64 | 151.0 | −i | |
| X-14626 | 36553 | LC/MS− | 4.80 | 288.8 | −i | Ion is doubly-charged; probable |
| | | | | | | sulfate |

Methods for the analysis of metabolites using LC-MS techniques are provided in U.S. Pat. Nos. 7,433,787 and 7,561,975, U.S. Patent Publication 20090017464 and using GC-MS techniques are provided in Lawton et al. (2008), Pharmacogenomics 9(4): 383-397, each of which is hereby incorporated herein by reference in its entirety.

The above table, Table 1: Unannotated Analytes, includes, for each listed Metabolite, the retention time (RT), retention index (RI), mass, and polarity obtained using the analytical methods described above. “Mass” refers to the mass of the C12 isotope of the parent ion used in quantification of the compound. “Polarity” indicates the polarity of the quantitative ion as being either positive (+) or negative (−). “Platform” indicates the compound was measured using GC/MS or LC/MS.

Methods of the present invention comprise clinico-metabolomic classifiers that are clinical evaluators including, but not limited to, patient demographics, exposure, symptoms, past medical history, results of physical examination, APACHE II score, SOFA score, DIC score, MELD score, and the development of ALI and ARDS. For example, clinical evaluators can comprise age, body temperature, mean arterial pressure, age, and platelet count

The term “sepsis” refers to a bloodstream infection with highly heterogeneous presentation, progression and high mortality. A patient presenting with sepsis is a patient who manifests the clinical criteria used to identify a patient with sepsis. Table 2 in the Examples, for example, provides selected clinical definitions for severe sepsis and septic shock. An aspect of the present invention is determining the sepsis status of an human, including whether or not the human has sepsis, and the risk for critical complications of sepsis, such as death. The present invention may provide a diagnosis of the sepsis status of a patient that is accurate more often than the clinical criteria and can rule out sepsis in certain patients who would otherwise be erroneously diagnosed with sepsis.

In aspects of methods of diagnosing sepsis in a human subject and methods of identifying a human subject at high risk of sepsis death, several reference characteristics are provided: (1) reference characteristics that were determined from subjects with sepsis, (2) reference characteristics that were determined from non-infected subjects with systemic inflammatory response (SIRS) criteria, (3) reference characteristics that were determined from subjects with sepsis that survive, (4) reference characteristics that were determined from subjects with sepsis that died within 28 days of presentation. These reference characteristics are used to make diagnoses and predictions related to sepsis status and likelihood of sepsis death.

A method of diagnosing sepsis in a human subject comprises, a) testing a subject for one or more clinico-metabolomic classifiers to determine the amount or level of one or more clinico-metabolomic classifiers in the subject to determine a value for the clinico-metabolomic classifier; b) comparing the value of one or more clinico-metabolomic classifiers from the subject to (i) a reference characteristic that was determined from the same clinico-metabolomic classifier obtained from subjects with sepsis, and to (ii) a reference characteristic that was determined from the same clinico-metabolomic classifier obtained from non-infected subjects with systemic inflammatory response (SIRS) or NIS criteria; and c) diagnosing the subject as having sepsis if the value of, or change in amount or level of, one or more clinico-metabolomic classifiers tested is found to be similar to the reference characteristic of subjects with sepsis.

For example, methods of diagnosing sepsis in a human subject may compare one or more of the subject's clinico-metabolomic classifier values to (i) a reference characteristic determined from the same clinico-metabolomic classifiers obtained from subjects with sepsis and to (ii) a reference characteristic determined from the same clinico-metabolomic classifier obtained from non-infected subjects with systemic inflammatory response (SIRS) criteria which may comprise measuring the amount or level of one clinico-metabolomic classifiers including, but not limited to, 10-undecenoate, 1-arachidonoyl-GPE (20:4), 1-methylimidazoleacetate, 1-palmitoleoylglycerophosphocholine (1-palmitoleoyl-GPC, C16:1), 1-palmitoyl-GPC (16:0), 2-hydroxybutyrate (AHB), 2-hydroxypalmitate, 3-(cystein-5-yl)acetaminophen, 3-methyl-2-oxovalerate, 4-acetamidobutanoate, 4-acetamidophenol, 5-dodecenoate (12:1n7), 5-oxoproline, acetylcarnitine (C2), allantoin, androsterone-sulfate, arabinose, bilirubin (E,E), caprate (10:0), caproate (6:0), C-glycosyltryptophan, citrulline, cortisol, creatine, dihydroepiandrosterone-sulfate (DHEAS), docosapentaenoate (DPA; 22:5n3), epiandrosterone-sulfate, erythronate, erythrose, fructose, galactonate, glutamate, glycerol-3-phosphate, heptanoate (7:0), hexadecanedioate (C16), indolepropionate, laurylcamitine, lysine, malate, maltose, mannose, N-acetylthreonine, palmitoylcarnitine (C16), pantothenate (Vitamin B5), phenol sulfate, phosphate, serine, tyrosine, uridine, X-07765, X-09789, X-11302, X-11381, X-11423, X-11793, X-11838, X-12029, X-12092, X-12442, X-12644, X-12644, X-12688, X-12794, X-12802, X-12860, X-14588, body temperature, mean arterial pressure, and bilirubin.

Methods of diagnosing sepsis may comprise measuring the amount or level of one clinico-metabolomic classifier, two clinico-metabolomic classifiers, three clinico-metabolomic classifiers, four clinico-metabolomic classifiers, five clinico-metabolomic classifiers, six clinico-metabolomic classifiers, or at least seven clinico-metabolomic classifiers. An example of a method where measurements of the amount or level of at least five clinico-metabolomic classifiers were made to result in a value for each clinico-metabolomic classifier, and in which each of the values is compared to reference characteristics for a particular sepsis status comprises measuring and comparing mean arterial pressure, body temperature, epiandrosterone-sulfate, X-09789, and X-11302.

A method of diagnosing sepsis in a human subject presenting with suspected sepsis, comprises a) testing a subject for one or more clinico-metabolomic classifiers to determine the amount or level of one or more clinico-metabolomic classifiers in the subject (a value); b) comparing the value of one or more clinico-metabolomic classifier from the subject to (i) a reference characteristic that was determined from the same clinico-metabolomic classifier obtained from subjects with sepsis who survive, and to (ii) a reference characteristic that was determined from the same clinico-metabolomic classifier obtained from non-infected subjects with systemic inflammatory response (SIRS or NIS) criteria; and c) diagnosing the subject as having sepsis if the value of, or change in amount or level of, one or more clinico-metabolomic classifiers tested is found within the reference characteristic of subjects with sepsis who survive.

For example, methods of diagnosing sepsis in a human subject may compare one or more of the subject's clinico-metabolomic classifier values to (i) a reference characteristic that was determined from the same clinico-metabolomic classifier obtained from subjects with sepsis that survive and to (ii) a reference characteristic that was determined from the same clinico-metabolomic classifier obtained from subjects with non-infected systemic inflammatory response (SIRS) criteria, which may comprise measuring the amount or level of one or more clinico-metabolomic classifiers including, but not limited to, 1-palmitoleoyl-GPC, 1-palmitoyl-GPC (16:0), 2-aminobutyrate, 3-methyl-2-oxovalerate, 4-acetamidobutanoate, 4-acetaminophen sulfate, 5-dodecenoate (12:1n7), acetylcarnitine, alanine, allantoin, androsterone sulfate, bilirubin (E,E), caprate, caproate, caprylate, citrate, citrulline, cortisol, creatine, creatinine, decanoylcarnitine, DHEAS, docosahexaenoate (DHA; 22:6n3), docosapentaenoate (DPA; 22:5n3), erythronate, fructose, glycerate, glycerol, glycine, heptanoate, hexadecanedioate, isoleucine, lactate, laurate, laurylcarnitine, leucine, maltose, myo-inositol, N-acetylornithine, octanoylcarnitine, phenylalanine, phosphate, proline, propionylcarnitine, pyruvate, serine, stearidonate (18:4n3), threonine, X-07765, X-11302, X-11421, X-11423, X-11793, X-11838, X-12206, X-12405, X-12465, X-12644, X-12794, X-12802, X-12855, age, body temperature, mean arterial pressure, age, and platelet count.

Methods of diagnosing sepsis may comprise measuring the amount or level of one clinico-metabolomic classifier, two clinico-metabolomic classifiers, three clinico-metabolomic classifiers, four clinico-metabolomic classifiers, five clinico-metabolomic classifiers, six clinico-metabolomic classifiers, or at least seven clinico-metabolomic classifiers. An example of a method where measurements of the amount or level of at least six clinico-metabolomic classifiers were made to result in a value for each clinico-metabolomic classifier, and in which the values are compared to reference characteristics for a particular sepsis status comprise measuring and comparing body temperature, alanine, glycine, acetylcarnitine, DHEAS, and X-11302.

A method of identifying a subject at high risk of sepsis death comprises a) testing a subject for one or more clinico-metabolomic classifiers to determine the amount or level of one or more clinico-metabolomic classifiers in the subject (a value); b) comparing the value of one or more clinico-metabolomic classifier from the subject to (i) a reference characteristic that was determined from the same clinico-metabolomic classifier obtained from subjects with sepsis that died, for example, within 28 days of presentation, and to (ii) a reference characteristic that was determined from the same clinico-metabolomic classifier obtained from subjects with sepsis that did not die, for example, within 28 days of presentation, or subjects with sepsis that survive; and c) diagnosing the subject as having a high risk of sepsis death if the value of, or change in amount or level of, one or more clinico-metabolomic classifiers tested is found within the reference characteristic of subjects with sepsis who die.

For example, methods of identifying a subject at high risk of sepsis death may compare the value of one or more clinico-metabolomic classifier from the subject to (i) a reference characteristic that was determined from the same clinico-metabolomic classifier obtained from subjects with sepsis that died, for example, within 28 days of presentation, and to (ii) a reference characteristic that was determined from the same clinico-metabolomic classifier obtained from subjects with sepsis that did not die, for example, within 28 days of presentation or subjects with sepsis that survive, may comprise measuring the amount or level of one or more clinico-metabolomic classifiers including, but not limited to, 1-arachidonoyl-GPC, 1-methylimidazole acetate, 1-palmitoleoyl-GPC, 2-methoxyacetaminophen sulfate, 3-(4-hydroxyphenyl)lactate (HPLA), 3-methoxytyrosine, 3-methylhistidine, 4-vinylphenol sulfate, 7-alpha-hydroxy-3-oxo-4-cholestenoate (7-HOCA), acetylcarnitine, alanine, androsterone sulfate, biliverdin, butyrylcarnitine, caproate, c-glycosyltryptophan, chenodeoxycholate, creatinine, decanoylcarnitine, dihydroxyacetone, epiandrosterone sulfate, gulono-1,4-lactone, heme, lactate, laurate, laurylcarnitine, N-acetylneuraminate, octanoylcarnitine, palmitoylcarnitine, piperine, piperine, propionylcarnitine, pyruvate, quinate, γ-tocopherol, X-02249, X-03056, X-06126, X-07765, X-10395, X-11255, X-11261, X-11302, X-11421, X-11444, X-11445, X-11450, X-11478, X-11538, X-11546, X-11809, X-11826, X-11843, X-11908, X-12051, X-12095, X-12100, X-12217, X-12405, X-12695, X-12742, X-12755, X-13553, X-14626, sodium, hematocrit, mean arterial pressure, and age.

Methods of identifying subjects at high risk for sepsis death may comprise measuring the amount or level of one clinico-metabolomic classifier, two clinico-metabolomic classifiers, three clinico-metabolomic classifiers, four clinico-metabolomic classifiers, five clinico-metabolomic classifiers, six clinico-metabolomic classifiers, or at least seven clinico-metabolomic classifiers. An example of a method where measurements of the amount or level of at least seven clinico-metabolomic classifiers were made to result in a value for each clinico-metabolomic classifier, and in which each of the values is compared to reference characteristics for a particular sepsis status comprises measuring and comparing sodium, lactate, age, hematocrit, HPLA, 3-methoxytyrosine, and X-11302.

A method of diagnosing or identifying a subject at high risk of sepsis death comprises a) testing a subject for one or more clinico-metabolomic classifiers to determine the amount or level of one or more clinico-metabolomic classifiers in the subject to determine a value for the clinico-metabolomic classifier; b) comparing the value of one or more clinico-metabolomic classifier from the subject (i) a reference characteristic that was determined from the same clinico-metabolomic classifier obtained from subjects with sepsis that died, for example, within 28 days of presentation, and to (ii) a reference characteristic that was determined from the same clinico-metabolomic classifier obtained from non-infected subjects with systemic inflammatory response (SIRS) or NIS criteria; and c) diagnosing the subject as having a high risk of sepsis death if the value of, or change in amount or level of, one or more clinico-metabolomic classifiers tested is found to be similar to the reference characteristic of subjects with high risk of sepsis death.

For example, methods of diagnosing or identifying a high risk of sepsis death in a human subject may compare one or more of the subject's clinico-metabolomic classifier values to (i) a reference characteristic determined from the same classifier obtained from subjects with sepsis that died, and to (ii) a reference characteristic determined from the same clinico-metabolomic classifier obtained from non-infected subjects with systemic inflammatory response (SIRS) criteria or (ii) a reference characteristic determined from the same clinico-metabolomic classifier obtained from subjects with sepsis, may comprise measuring the amount or level of 3-(4-hydroxyphenyl)lactate (HPLA), 3-methoxytyrosine, piperine, 3-methylhistidine, lactate, caproate, acetylcarnitine, octanoylcarntine, decanoylcarnitine, propionylcarnitine, creatinine, γ-tocopherol, chenodeoxycholate, X-10395, X-11261, X-11538, X-11908, X-12095, X-12100, X-12775, X-11302, X-13553, heme, sodium, hematocrit, mean arterial pressure, body temperature, age, and respiratory rate.

Methods of diagnosing sepsis death may comprise measuring the amount or level of one clinico-metabolomic classifier, two clinico-metabolomic classifiers, three clinico-metabolomic classifiers, four clinico-metabolomic classifiers, five clinico-metabolomic classifiers, six clinico-metabolomic classifiers, or at least seven clinico-metabolomic classifiers. An example of a method where measurements of the amount or level of at least six clinico-metabolomic classifiers were made to result in a value for each clinico-metabolomic classifier, and in which each of the values is compared to reference characteristics for a particular sepsis status comprises measuring and comparing mean arterial pressure, sodium, hematocrit, HPLA, piperine, and γ-tocopherol.

The methods disclosed herein may measure the amount or level or one or many clinico-metabolomic classifiers, from one to all or some of the list of clinico-metabolomic classifiers shown in FIG. 14, or disclosed herein. Measurements and comparisons may be made for 1 to 7 clinico-metabolomic classifiers, from 1-8, from 1-9, from 1-10, from 1-20, from 1-30, from 1-40, from 1-50, from 1-60, from 1-70, from 1-100, and more, including numbers in the ranges there between.

A method for determining a treatment regiment of a subject at high risk of sepsis death may comprise (a) determining whether that the subject has a high risk of sepsis death according to a method disclosed herein; and (b) performing a series of medical procedures that lessen the subject's risk of sepsis death. For example, a method for determining a treatment regimen of a subject at high risk of sepsis death may comprise a method of identifying a subject at high risk of sepsis death comprising: comparing the value of at least one clinico-metabolomic classifier from the subject to (i) a reference characteristic determined from the same clinico-metabolomic classifier obtained from subjects with sepsis that died, for example, within 28 days of presentation, and to (ii) a reference characteristic determined from the same clinico-metabolomic classifier obtained from subjects with sepsis that did not die, for example, within 28 days of presentation, wherein the clinico-metabolomic classifier comprises an analyte or a clinical evaluator, and c) diagnosing the subject as having a high risk of sepsis death if the value of, or change in amount or level of, one or more clinico-metabolomic classifiers tested is found to be similar to the reference characteristic of subjects with a high risk of sepsis death.

Methods for monitoring the progress of treatment of a subject with sepsis, or a subject at high risk of sepsis death may comprise repeating the methods steps of a disclosed diagnosis method, such as measuring the amount or level of one or more clinico-metabolomic classifiers, which may be the same clinico-metabolomic classifiers or different ones from those previously measured, and comparing the values obtained for each clinico-metabolomic classifier to reference characteristics for sepsis status. Changes in the clinico-metabolomic classifiers may indicate changes in the subject's condition and sepsis status.

A series of medical procedures that lessen the subject's risk of sepsis death may comprise admitting the patient to the Intensive Care Unit, providing early goal directed therapy (EGDT), administering activated protein C (e.g., Xigris®), providing intensive glycemic control, administering hyperbaric or supplemental oxygen, administering exogenous steroids, or other medical procedures known in the art.

A method of monitoring the progress of treatment of a patient suspected of having sepsis or a patient diagnosed as having sepsis may comprise serial application of the methods disclosed above, for example, repeating a method one or more times, over a period of one, two, three days, weekly or at frequent or infrequent intervals. For example, the method of monitoring the progress of treatment of a patient suspected of having sepsis or a patient diagnosed as having sepsis can comprise a method of identifying a subject at high risk of sepsis death comprising a) testing a subject for one or more clinico-metabolomic classifiers to determine the amount or level of one or more clinico-metabolomic classifiers in the subject to determine a value for the clinico-metabolomic classifier; b) comparing the value of one or more clinico-metabolomic classifier from the subject to (i) a reference characteristic that was determined from the same clinico-metabolomic classifier obtained from human subjects with sepsis that died, for example, within 28 days of presentation, and to (ii) a reference characteristic that was determined from the same clinico-metabolomic classifier obtained from non-infected subjects with systemic inflammatory response (SIRS) or NIS criteria; or to (ii) a reference characteristic that was determined from the same clinico-metabolomic classifier obtained from subjects with sepsis that did not die, and c) diagnosing the subject as having a high risk of sepsis death if the value of, or change in amount or level of, one or more clinico-metabolomic classifiers tested is found to be similar to the reference characteristic of subjects with sepsis death.

For disclosed methods, the value of the clinico-metabolomic classifiers is determined by measuring/quantifying the value of the classifier. The art will recognize that for some classifiers, both the reagent for detecting and a methods for quantitating are needed. For other classifiers, only the methods for quantitating is needed. For example, analytes, which can be measured in blood, plasma, or serum, can be measured by methods known in the art including but not limited to quantitative mass spectrometry assays and clinical chemistry assays (e.g., using conventional clinical chemistry analyzer). Clinical evaluators can be measured by methods that are standard in the art including but not limited to standards that are employed in emergency rooms and hospitals across the country.

Disclosed methods of diagnosing sepsis in a human subject and methods of identifying a human subject at high risk of sepsis death may comprise various combinations of clinico-metabolomic classifiers, for example, at least six clinico-metabolomic classifiers. For example, for the following forty-four (44) clinico-metabolomic classifiers—10-undeccnoate, 1-palmitolcoyl-GPC, 3-(4-hydroxyphenyl)lactatc (HPLA), 3-methoxytyrosine, 3-methylhistidine, acetylcamitine, age, alanine, androsterone-sulfate, billirubin, citrulline, creatine, creatinine, decanoylcamitine, dihydroepiandrosterone-sulfate (DHEAS), epiandrosterone-sulfate, erythrose, fructose, glutamate, glycerol-3-phosphate, glycine, hematocrit, heme, indolepropionate, lactate, laurylcamitine, lysine, maltose, mean arterial pressure, piperine, platelet count, pyruvate, respiratory rate, serine, sodium, temperature, threonine, X-09789, X-10395, X-11302, X-11444, X-11538, X-11908, and γ-tocopherol—yield approximately 5,082,517,440 different combinations comprising six unique clinico-metabolomic classifiers.

Disclosed herein is a kit for diagnosing sepsis comprising: a reagent for detecting one or more clinico-metabolomic classifiers including, but not limited to, 1-palmitoleoyl-GPC, 1-palmitoyl-GPC (16:0), 2-aminobutyrate, 3-methyl-2-oxovalerate, 4-acetamidobutanoate, 4-acetaminophen sulfate, 5-dodecenoate (12:1n7), acetylcarnitine, alanine, allantoin, androsterone sulfate, bilirubin (E,E), caprate, caproate, caprylate, citrate, citrulline, cortisol, creatine, creatinine, decanoylcarnitine, DHEAS, docosahexaenoate (DHA; 22:6n3), docosapentaenoate (DPA; 22:5n3), erythronate, fructose, glycerate, glycerol, glycine, heptanoate, hexadecanedioate, isoleucine, lactate, laurate, laurylcamitine, leucine, maltose, myo-inositol, N-acetylornithine, octanoylcarnitine, phenylalanine, phosphate, proline, propionylcarnitine, pyruvate, serine, stearidonate (18:4n3), threonine, X-07765, X-11302, X-11421, X-11423, X-11793, X-11838, X-12206, X-12405, X-12465, X-12644, X-12794, X-12802, and X-12855.

Disclosed methods can utilize at least two clinico-metabolomic classifiers, or at least three clinico-metabolomic classifiers, or at least four clinico-metabolomic classifiers, or at least five clinico-metabolomic classifiers, or at least six clinico-metabolomic classifiers, or at least seven clinico-metabolomic classifiers. An example of a method using the clinico-metabolomic classifiers can measure and compare alanine, glycine, acetylcarnitine, DHEAS, and X-11302. An example of a method using the clinico-metabolomic classifiers can measure and compare epiandrosterone-sulfate, X-09789, and X-11302

Kits for diagnosing sepsis disclosed herein can be utilized in the methods disclosed herein. For example, using the reagent for detecting a clinico-metabolomic classifier, the value of the subject's clinico-metabolomic classifier is compared to a first reference characteristic that was determined from same clinico-metabolomic classifier obtained from human subjects with sepsis and to a second reference characteristic that was determined from the same clinico-metabolomic classifier obtained from subjects with non-infected systemic inflammatory response criteria, and diagnosing the sepsis status.

Also disclosed herein is a kit for diagnosing a subject at high risk for sepsis death comprising a reagent for detecting a clinico-metabolomic classifier including, but not limited to, 1-arachidonoyl-GPC, 1-methylimidazole acetate, 1-palmitoleoyl-GPC, 2-methoxyacetaminophen sulfate, 3-(4-hydroxyphenyl)lactate (HPLA), 3-methoxytyrosine, 3-methylhistidine, 4-vinylphenol sulfate, 7-alpha-hydroxy-3-oxo-4-cholestenoate (7-HOCA), acetylcarnitine, alanine, androsterone sulfate, biliverdin, butyrylcarnitine, caproate, c-glycosyltryptophan, chenodeoxycholate, creatinine, decanoylcarnitine, dihydroxyacetone, epiandrosterone sulfate, gulono-1,4-lactone, heme, lactate, laurate, laurylcarnitine, N-acetylneuraminate, octanoylcarnitine, palmitoylcarnitine, piperine, piperine, propionylcarnitine, pyruvate, quinate, γ-tocopherol, X-02249, X-03056, X-06126, X-07765, X-10395, X-11255, X-11261, X-11302, X-11421, X-11444, X-11445, X-11450, X-11478, X-11538, X-11546, X-11809, X-11826, X-11843, X-11908, X-12051, X-12095, X-12100, X-12217, X-12405, X-12695, X-12742, X-12755, X-13553, X-14626 sodium, hematocrit, mean arterial pressure, and age.

Disclosed methods can utilize at least two clinico-metabolomic classifiers, or at least three clinico-metabolomic classifiers, or at least four clinico-metabolomic classifiers, or at least five clinico-metabolomic classifiers, or at least six clinico-metabolomic classifiers, or at least seven clinico-metabolomic classifiers. An example of a method using the clinico-metabolomic classifiers can measure and compare sodium, lactate, HPLA, 3-methoxytyrosine, and X-11302. Another example of a method using the clinico-metabolomic classifiers can measure and compare sodium, HPLA, piperine, and γ-tocopherol.

Disclosed kits can comprise various written instructions, vessels, buffers, reagents, and other compositions that are necessary to determine the value of the subject's at least one clinico-metabolomic classifier.

For disclosed kits, the value of the clinico-metabolomic classifiers may be determined by measuring or quantifying the level or amount of a clinico-metabolomic classifier. The art will recognize that for some clinico-metabolomic classifiers, both the reagent for detecting and a methods for quantitating are needed. For other clinico-metabolomic classifiers, only the methods for quantitating is needed. For example, analytes, which can be obtained from samples of bodily fluids such as saliva, nasal secretions, blood, plasma, serum or others known to those skilled in the art, can be measured by methods known in the art including, but not limited to, quantitative mass spectrometry assays and clinical chemistry assays (e.g., using a conventional clinical chemistry analyzer). Clinical evaluators can be measured by methods that arc standard in the art including, but not limited to, methods that are employed by physicians, nurses and other healthcare personnel in healthcare facilities such as emergency rooms and hospitals.

As aspect of the present invention comprises comparing of the value of the subject's clinico-metabolomic classifier to at least one reference characteristic using statistical and comparative methods of predictive modeling. A computer software program, such as JMP Genomics 4.0, may be used. To create a predictive model, data is collected for the clinico-metabolomic classifiers, a statistical model is formulated, and predictive modeling provides the resulting reference characteristic. Predictive modeling can be performing in a variety of ways known in the art including but not limited to (1) discriminant analysis, (2) general linear model selection (GLMS), (3) logistic regression, (4) partition trees, and (5) beta process factor analysis and group probit regression (BPFA-GPR). For example, in the disclosed methods and kits, predictive modeling using partition trees yielded a classifier group comprising sodium, lactate, age, hematocrit, HPLA, 3-methoxytyrosine, and X-11302. (Table 7). In the disclosed methods and kits, logistic regression of the sepsis prognostic indices yielded optimal classification of patients that would survive or die. (Table 8). In the disclosed methods and kits, predictive modeling using partition trees yielded a classifier group comprising mean arterial pressure, temperature, epiandrosterone-sulfate, X-09789, and X-11302. (Table 9). In the disclosed methods and kits, predictive modeling using BPFA-GPR yielded a classifier group comprising creatinine, 4-vinylphenol sulfate, c-glycosyltryptophan, X-11261, X-12095, x-12100, and X-13553. (Table 10).

The disclosed methods and kits can be performed by comparing of the value of the subject's clinico-metabolomic classifier to the same clinico-metabolomic classifier in at least one reference characteristic. One of skill in the art will recognize that there are multiple ways for developing a reference characteristic for a clinico-metabolomic classifier against which the value of a clinical measurement for that clinico-metabolomic classifier can be compared. One of skill in the art will also recognize that the choice of ways can be based on the intended use of the information to be obtained by the comparison. For example, ranges can be generated from a population of one (1) subject, wherein the range comprises the value of a clinico-metabolomic classifier at multiple time points. Multiple time points can include time points before and after various treatments. Alternatively, a reference characteristic can be generated from a population of multiple subjects, wherein the reference characteristic comprises the average value of the clinico-metabolomic classifier at a single time point. For example, Table 4 lists the means and standard errors for a variety of clinico-metabolomic classifiers, including sodium, hematocrit, respiratory rate, temperature, mean arterial pressure, and platelet count. Table 4 lists the values of these clinico-metabolomic classifiers for several populations of subjects including subjects with uncomplicated sepsis, subjects with severe sepsis, subjects with septic shock, and subjects with sepsis that died. Using a population of multiple subjects can also generate average values for clinico-metabolomic classifiers at multiple time points. Reference characteristics for disclosed methods are exemplified in the Examples.

The present invention comprises methods for determining one or more reference characteristics for sepsis status, including but not limited to, sepsis, high risk of death from sepsis or non-sepsis (NIS or SIRS). The method can be operational with numerous other general purpose or special purpose computing system environments or configurations. Examples of well known computing systems, environments, and/or configurations that may be suitable for use with the system and method include, but are not limited to, personal computers, server computers, laptop devices, and multiprocessor systems. Additional examples include set top boxes, programmable consumer electronics, network PCs, minicomputers, mainframe computers, distributed computing environments that include any of the above systems or devices, and the like.

The processing of the disclosed method can be performed by software components. The disclosed method may be described in the general context of computer-executable instructions, such as program modules, being executed by one or more computers or other devices. Generally, program modules include computer code, routines, programs, objects, components, data structures, etc. that perform particular tasks or implement particular abstract data types. The disclosed method may also be practiced in grid-based and distributed computing environments where tasks are performed by remote processing devices that are linked through a communications network. In a distributed computing environment, program modules may be located in both local and remote computer storage media including memory storage devices. The method may be practiced utilizing firmware configured to perform the methods disclosed herein in conjunction with system hardware.

The methods and systems of the present invention can employ Artificial Intelligence techniques such as machine learning and iterative learning. Examples of such techniques include, but are not limited to, expert systems, case based reasoning, Bayesian networks, behavior based AI, neural networks, fuzzy systems, evolutionary computation (e.g., genetic algorithms), swarm intelligence (e.g., ant algorithms), and hybrid intelligent systems (e.g., Expert inference rules generated through a neural network or production rules from statistical learning).

The method disclosed herein can be implemented via a general-purpose computing device in the form of a computer. The components of the computer can include, but are not limited to, one or more processors or processing units, a system memory and a system bus that couples various system components including the processor to the system memory.

The system bus represents one or more of several possible types of bus structures, including a memory bus or memory controller, a peripheral bus, an accelerated graphics port, and a processor or local bus using any of a variety of bus architectures. By way of example, such architectures can include an Industry Standard Architecture (USA) bus, a Micro Channel Architecture (MCA) bus, an Enhanced ISA (EISA) bus, a Video Electronics Standards Association (VESA) local bus, and a Peripheral Component Interconnects (PCI) bus also known as a Mezzanine bus. This bus, and all buses specified in this description can also be implemented over a wired or wireless network connection. The bus, and all buses specified in this description can also be implemented over a wired or wireless network connection and each of the subsystems, including the processor, a mass storage device, an operating system, a network adapter, patient data, clinico-metabolic classifer data, characteristic data, system memory, an Input/Output Interface, a display adapter, a display device, and a human machine interface, can be contained within one or more remote computing devices at physically separate locations, connected through buses of this form, in effect implementing a fully distributed system.

The computer typically includes a variety of computer readable media. Such media can be any available media that is accessible by the computer and includes both volatile and non-volatile media, removable and non-removable media. The system memory includes computer readable media in the form of volatile memory, such as random access memory (RAM), and/or non-volatile memory, such as read only memory (ROM). The system memory typically contains data such as patient data and metabolite data, and/or program modules such as operating system and metabolite software that are immediately accessible to and/or are presently operated on by the processing unit.

The computer may also include other removable/non-removable, volatile/non-volatile computer storage media. A mass storage device can provide non-volatile storage of computer code, computer readable instructions, data structures, program modules, and other data for the computer. For example, a mass storage device can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules can be stored on the mass storage device, including by way of example, an operating system and software relating to patient data, clinico-metabolomic classifier data, and/or characteristic data. Each of the operating system and software (or some combination thereof) may include elements of the programming and the relevant software. Patient data, clinico-metabolic classifer data, and characteristic data can also be stored on the mass storage device. Patient data, clinico-metabolic classifer data, and characteristic data can be stored in any of one or more databases known in the art. Examples of such databases include, DB2®, Microsoft® Access, Microsoft® SQL Server, Oracle®, mySQL, PostgreSQL, and the like. The databases can be centralized or distributed across multiple systems.

A user can enter commands and information into the computer via an input device. Examples of such input devices include, but are not limited to, a keyboard, pointing device (e.g., a “mouse”), a microphone, a joystick, a serial port, a scanner, and the like. These and other input devices can be connected to the processing unit via a human machine interface that is coupled to the system bus, but may be connected by other interface and bus structures, such as a parallel port, game port, or a universal serial bus (USB).

A display device can also be connected to the system bus via an interface, such as a display adapter. A computer can have more than one display adapter and a computer can have more than one display device. For example, a display device can be a monitor, an LCD (Liquid Crystal Display), or a projector. In addition to the display device, other output peripheral devices can include components such as speakers and a printer which can be connected to the computer via Input/Output Interface.

The computer can operate in a networked environment using logical connections to one or more remote computing devices. By way of example, a remote computing device can be a personal computer, portable computer, a server, a router, a network computer, a peer device or other common network node, and so on. Logical connections between the computer and a remote computing device can be made via a local area network (LAN) and a general wide area network (WAN). Such network connections can be through a network adapter. A network adapter can be implemented in both wired and wireless environments. Such networking environments are commonplace in offices, enterprise-wide computer networks, intranets, and the Internet.

For purposes of illustration, application programs and other executable program components such as the operating system are illustrated herein as discrete blocks, although it is recognized that such programs and components reside at various times in different storage components of the computing device, and are executed by the data processor(s) of the computer. An implementation of application software may be stored on or transmitted across some form of computer readable media. Computer readable media can be any available media that can be accessed by a computer. By way of example, and not limitation, computer readable media may comprise “computer storage media” and “communications media.” “Computer storage media” include volatile and non-volatile, removable and non-removable media implemented in any method or technology for storage of information such as computer readable instructions, data structures, program modules, or other data. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

While this invention has been described in connection with preferred embodiments and specific examples, it is not intended that the scope of the invention be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

The present invention, methods, compositions and kits are disclosed and described herein. It is to be understood that the methods, compositions and kits are not limited to specific synthetic methods, specific components, or to particular compositions. It is also to be understood that the terminology used herein is for the purpose of describing particular embodiments only and is not intended to be limiting.

Disclosed are components that can be used to perform the disclosed systems and methods. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present systems and methods may be understood more readily by reference to the following detailed description and the Examples included therein and to the Figures and their previous and following descriptions.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances in which said event or circumstance occurs and instances in which it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Ranges can be expressed herein as from “about” one particular value, and/or to “about” another particular value. When such a range is expressed, another embodiment includes—from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The term “sepsis survivors” refers to patients who have survived uncomplicated sepsis, sepsis shock (i.e., patients with definite infections who progressed to septic shock by day 3 post-presentation, but did not die by day 28 post-presentation), or severe sepsis (i.e., patients with definite infections who progressed to severe sepsis by day 3 post-presentation, but did not develop septic shock or death by day 28 post-presentation).

The term “sepsis death” refers to patients with infections, who had sepsis, who died by day 28 post-presentation.

As used herein, “reference range” refers to a range of values against which the value of a particular unknown sample is compared. The high and low values in a reference range can be empirically determined from a number of samples of known origin. Alternatively, the values in the reference range can be determined using predictive modeling. Depending on what condition the reference range identifies, the high and low values of the range can differ widely from each other (e.g., by orders of magnitude) or can differ minimally (e.g., by less than a whole number).

As used herein, the terms “patient”, “human”, “subject” have their usual and accepted meanings and are referred to interchangeably.

All patents, patent applications and references included herein are specifically incorporated by reference in their entireties.

It should be understood, of course, that the foregoing relates only to preferred embodiments of the present invention and that numerous modifications or alterations may be made therein without departing from the spirit and the scope of the invention as set forth in this disclosure.

The present invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLES

The following examples are put forth so as to provide those of ordinary skill in the art with a complete disclosure and description of how the compounds, compositions, articles, devices, apparatuses, and methods taught herein are made and evaluated, and are intended to be purely exemplary and are not intended to limit the scope of the methods and systems. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric.

### Example 1

### Identification of Capsod Study Sites and Patients

Inclusion criteria were presentation at the emergency department with known or suspected acute infection and presence of at least two of the systemic inflammatory response syndrome (SIRS) criteria (Bone et al., 1992). Exclusion criteria were age less than six years, pregnancy, presence of an imminently terminal co-morbid condition, recent treatment with an antibiotic for a bacterial or fungal infection, Human immunodeficiency virus (HIV) infection with a last known CD4 count of <50 mm³, acute presence of a cerebral vascular event, active gastrointestinal hemorrhage, seizure episode, drug overdose, burn injury, trauma or participation in an ongoing clinical trial, as previously described (Glickman et al., 2010). Patients were enrolled from 2005 through 2009 in emergency departments at each institution and written, informed consent was obtained by all study participants or their legal designates.

TABLE 2
CLINICAL EVALUATORS FOR SEVERE SEPSIS AND SEPTIC SHOCK

| Dysfunction | Measure | Range |
|---|---|---|
| Cardiovascular | Arterial systolic | ≦90 mmHG (≧18 years) or |
| dysfunction^{1,2} | pressure | ≦85 mmHG (13-17 years) |
| | Mean arterial pressure | ≦70 mmHG (≧18 years) |
| | Vasopressors | dopamine (≧5 μg kg/min) |
| | | norepinephrine, epinephrine or phenylephrine (any dose) |
| Renal dysfunction¹ | Urine output | <0.5 mL/kg/h |
| Respiratory | PaO₂/FiO₂³ | ≦250 |
| dysfunction | | ≦200 (if only SS criterion met, or if the lung is the |
| | | suspected site of infection) |
| Hematological | Platelet count | <80,000 (≧18 years of age) or <100,000 (≦17 years of |
| dysfunction | | age) or 50% decrease over 3 d |
| Metabolic | plasma pH | ≦7.3 (≧18 years of age) |
| dysfunction | | ≦7.28 (≦17 years of age) |
| | Base deficit (BD) + | 18 years or older, BD ≧ 5.0 mEq/L, Lactate > 1.5x |
| | lactate | upper limit of normal reporting lab |
| | CO₂ + lactate | 17 years or younger, CO₂ < 17 mEg/L, Lactate > 1.5x |
| | | upper limit of normal reporting lab |

¹Despite adequate fluid resuscitation or adequate intravascular volume
²If 17 or under, then must meet two cardiovascular dysfunction criteria
³If SaO₂ only, then PaO₂ calculated from standard oxyhemaglobin dissociation curve with assumption of

### Example 2

### Collection of Clinical Data

Patient demographics, exposure, symptoms, past medical history, results of physical examination, APACHE II score, SOFA score, DIC score, MELD score, development of ALI and ARDS and treatment were recorded at enrollment (t₀) and at 24 hours (t₂₄) by a nurse practitioner or physician using online electronic data capture (Prosanos, La Jolla, Calif.), as previously described (Glickman et al., 2010; Dellinger et al., 2008; Knaus et al., 1985; Vincent et al., 1996). Microbiologic evaluation was as indicated clinically together with urinary pneumococcal and legionella antigen tests. Finger-stick lactate values were obtained. Other laboratory, microbiology and radiographic tests were ordered by the treating physician according to standards of care. Following patient discharge or death, charts were reviewed and largest deviations of clinical and laboratory parameters from normal were recorded, together with occurrence of outcome measures, microbiologic results, treatment and time-to-events.

### Example 3

### Collection of Plasma

Blood was collected in bar-coded EDTA-plasma tubes at enrollment (t₀) and the following day (t₂₄). The blood was incubated on ice until centrifuged (within 4 hours), the plasma was separated. Aliquots were stored at −80° C.

### Example 4

### Audit of Clinical Data Audit and Selection of Discovery Cohort

To determine whether presenting symptoms and signs were due to infection, a board-certified study physician adjudicated independently the records from all enrollees (1152). The board-certified study physician also identified etiologic agent, site of infection, patient outcomes, and times-to-outcomes. Complex patient records were reviewed by a second physician. Approximately 10% of records were audited for correctness by a third physician. Based on a kappa for infection versus no infection of 0.82 (95% confidence interval 0.62-1.00), agreement among the physicians was high. Patients were assigned to categories, as described (Glickman et al., 2010): (1) definite infection, causative organism identified; (2) definite infection, causative organism uncertain; (3) indeterminate, infection possible; (4) no evidence of infection; and (5) no evidence of infection and diagnosis of a non-infectious process that accounted for SIRS. 150 patients were selected from the definite infection and non-infection categories for plasma metabolome and proteome analyses (Bone et al., 1992; Bone et al., 1989; Balk 2000). The breakdown of the 150 patients is as follows: (1) non-infected patients with SIRS (NIS, n=29); (2) uncomplicated sepsis (UCS; n=27) (Bone et al., 1992; Bone et al., 1989; Balk 2000); (3) severe sepsis (SS; patients with definite infections who progressed to severe sepsis by day 3, but did not develop septic shock or death by day 28, n=25, see, e.g., Table 2) (Bone et al., 1992; Bone et al., 1989; Balk 2000); (4) septic shock (SShock; patients with definite infections who progressed to septic shock by day 3 but did not die by day 28, n=38, see, e.g., Table 2) (Bone et al., 1992; Bone et al., 1989; Balk 2000); and (5) sepsis deaths (SD; patients with definite infections who died by day 28, n=31). Patients with definite infections were further selected to maximize the number caused by Escherichia coli, Staphylococcus aureus, and Streptococcus pneumoniae, and to provide a wide range of APACHE II scores. Within these constraints, groups were matched for age, race, sex, and enrollment site (Table 3). Fifty two additional sepsis survivors and sepsis deaths were selected and matched for replication t₀ and t₂₄ studies.

TABLE 3
MATCHED PATIENT DEMOGRAPHICS

| | | African- | | | | | | |
|---|---|---|---|---|---|---|---|---|
| | | American | White | Male | HFHS | Duke | APACHE | |
| | n | (%) | (%) | (%) | (%) | (%) | II | Age |
| Definite infection: Agent | 103 | 66.3 | 27.2 | 59.2 | 73.8 | 26.0 | 17.6 ± 7.9 | 57.3 ± 17.3 |
| identified | | | | | | | | |
| Definite infection: Agent | 18 | 61.0 | 39.0 | 50.0 | 66.7 | 33.3 | 22.2 ± 10.1 | 70.6 ± 17.4 |
| unidentified | | | | | | | | |
| Definite non-infection | 29 | 72.4 | 24.1 | 41.4 | 79.3 | 20.7 | 17.6 ± 7.2 | 65.8 ± 13.6 |
| (SIRS positive)¹ | | | | | | | | |
| Uncomplicated Sepsis | 27 | 66.7 | 29.6 | 66.7 | 59.3 | 40.7 | 11.6 ± 5.7 | 53.8 ± 15.4 |
| Sepsis Death^{1,3} | 31 | 74.2 | 22.6 | 55.2 | 87.1 | 12.9 | 23.5 ± 9.0 | 68.8 ± 16.7 |
| Septic Shock² | 38 | 52.6 | 36.8 | 50.0 | 65.8 | 34.2 | 19.4 ± 7.8 | 58.6 ± 18.5 |
| Severe Sepsis² | 25 | 76.0 | 24.0 | 64.0 | 84.0 | 20.0 | 18.6 ± 5.6 | 55 ± 16.8 |
| APACHE II⁴ ≧ 20 | 44 | 77.3 | 18.2 | 50.0 | 81.8 | 18.2 | 25.8 ± 6.1 | 67.5 ± 16.2 |
| APACHE II ≦ 19 | 67 | 61.2 | 34.3 | 58.2 | 70.1 | 29.9 | 13.2 ± 4.7 | 57.1 ± 17.0 |
| S. aureus⁵ | 27 | 70.4 | 29.6 | 77.7 | 62.9 | 37.0 | 16.7 ± 8.8 | 52.4 ± 15.3 |
| S. pneumoniae⁵ | 31 | 71.0 | 19.4 | 51.6 | 87.1 | 12.9 | 17.6 ± 7.1 | 57.4 ± 17.2 |
| E. Coli⁵ | 16 | 68.8 | 25.0 | 62.5 | 56.3 | 43.8 | 16.8 ± 8.8 | 57.6 ± 16.0 |
| Community acquired | 44 | 63.6 | 27.3 | 65.9 | 81.8 | 18.2 | 20.7 ± 9.5 | 60.7 ± 18.0 |
| pneumonia | | | | | | | | |

¹Constrained - little or no choice; ≧2 SIRS;
²Day 0-3
³Day 1-28
⁴Acute Physiology And Chronic Health Evaluation Score II
⁵Bacteremia

### Example 5

### Preparation of Metabolite Samples

Plasma samples were thawed on ice and 100 μL was extracted using an automated MicroLab STAR® system (Hamilton Company, Salt Lake City, Utah), as described (Lawton et al., 2008). To assess variability and sensitivity in the measurement of all consistently detected chemicals, a well characterized human plasma pool (“Matrix”, MTRX) was also included as a technical replicate. A single solvent extraction was performed with 400 μL of methanol, containing the recovery standards tridecanoic acid, fluorophenylglycine, chlorophenylalanine, and d6-cholesterol, by shaking for two minutes using a Geno/Grinder 2000 (Glen Mills Inc., Clifton, N.J.).

After extraction, the sample was centrifuged, the supernatant removed and split into four equal aliquots: two (2) for liquid chromatography/mass-spectrometry (LC/MS), one (1) for gas chromatography/mass-spectrometry (GC/MS), and one (1) reserve aliquot. Aliquots were dried under vacuum overnight on a TurboVap® (Zymark). Samples were maintained at 4° C. throughout the extraction process. For LC/MS analysis, aliquots were reconstituted in 0.1% formic acid for positive ion LC/MS or in 6.5 mM ammonium bicarbonate pH 8.0 for negative ion LC/MS. For GC/MS analysis, aliquots were derivatized using equal parts N,O-bistrimethylsilykrifluoroacetamide and a mixture of acetonitrile:dichloromethane:cyclohexane (5:4:1) with 5% triethylamine at 60° C. for 1 hour. The derivatization mixture also contained a series of alkyl benzenes that served as retention time markers.

LC/MS was carried out using an Acquity HPLC (Waters Corporation, Milford, Mass.) coupled to a linear trap quadrupole (LTQ) mass spectrometer (Thermo Fisher Scientific Inc., Waltham, Mass.) equipped with an electrospray ionization source. Two (2) separate LC/MS injections were performed on each sample. The first LC/MS injection was optimized for positive ions and the second LC/MS injection was optimized for negative ions. The mobile phase for positive ion analysis consisted of 0.1% formic acid in H₂O (solvent A) and 0.1% formic acid in methanol (solvent B). The mobile phase for negative ion analysis consisted of 6.5 mM ammonium bicarbonate, pH 8.0 (solvent A) and 6.5 mM ammonium bicarbonate in 95% methanol (solvent B). The acidic and basic extracts were monitored for positive and negative ions, respectively, using separate acid/base dedicated 2.1×100 mm Waters BEH C18 1.7 μm particle columns heated to 40° C. The extracts were loaded via a Waters Acquity autosampler and gradient eluted (0% B to 98% B, with an 11 minute run time) directly into the mass spectrometer at a flow rate of 350 μL/min. The LTQ alternated between full scan mass spectra (99-1000 m/z) and data dependent MS/MS scans, which used dynamic exclusion.

Derivatized samples were analyzed on a Thermo-Finnigan Trace DSQ fast-scanning single-quadrupole MS set at unit mass resolving power. The GC column was 20 m×0.18 mm with 0.18 μm film phase consisting of 5% phenyldimethyl silicone. The temperature program ramped from 60° C. to 340° C., with helium as the carrier gas. The MS was operated using electron impact ionization with a 50-750 amu scan range, tuned and calibrated daily for mass resolution and mass accuracy. Samples were randomized to avoid group block effects and were analyzed over five days. Six MTRX aliquots, an internal standard sample (see below) and control samples (without plasma extract) were included in each run.

Metabolites were identified by automated comparison to a reference library of purified external standards using Metabolon software developed for creating library entries from known chemical entities with automatic fitting of reference to experimental spectra. Peaks that eluted from the LC or GC methods were compared to the library at a particular retention time and associated spectra for that metabolite. An internal standard comprising of 30 organic molecules was used in the GC and LC methods to calibrate retention times of metabolites across all samples. Platform variability was determined by calculating the median relative standard deviation for the internal standard compounds. Overall variability (including sample preparation) was determined by the median RSD for 261 metabolites present in all MTRX samples. Peptides were identified using standard tandem mass spectrometry sequencing.

Raw area counts for each metabolite in each sample were normalized to correct for variation resulting from instrument inter-day tuning differences. For each metabolite, the raw area counts were divided by the median value for each run-day, therefore setting the medians to 1 for each run. This preserved variation between samples, but allowed metabolites of widely different raw peak areas to be compared on a similar graphical scale. Missing values were imputed with the observed minimum after normalization. However, metabolites with missing values in >50% of the samples were excluded from analysis.

### Example 6

### Preparation of Proteomic Sample and Analysis by Mass Spectrometer

Plasma samples were thawed on ice. The most abundant proteins (albumin, IgG, fibrinogen, transferrin, IgA, IgM, haptoglobin, α2-macroglobulin, α1-acid glycoprotein, α1-antitrypsin and apolipoprotein A-I and A-II) were removed using Seppro IgY12 Columns (GenWay Biotech Inc., San Diego, Calif.). Column flow-throughs were denatured by 8M urea, reduced by triethylphosphine, alkylated by iodoethanol and digested by trypsin, as described (Hale et al., 2004). Tryptic digests (˜20 μg) were analyzed using a Thermo-Fisher Scientific linear ion-trap mass spectrometer (LTQ) coupled with a Surveyor HPLC system. Peptides were separated on a C18 reverse phase column (i.d.=2.1 mm, length=50 mm) with a flow rate of 200 μL/min and eluted with a gradient from 5% to 45% acetonitrile developed over 120 min. All injections were randomized and the instrument was operated by the same operator for the study. Data were collected in the triple-play mode (MS scan, zoom scan and MS/MS scan). Data were filtered and analyzed by a proprietary algorithm (Higgs et al., 2005; Higgs et al., 2007). Database searches against the International Protein Index (IPI) human database and the non-Redundant-Homo Sapiens database were carried out using both the X!Tandem and SEQUEST algorithms (Yates et al., 1995; Craig and Beavis, 2004). Observed peptide MS/MS spectrum and theoretically derived spectra were used to assign quality scores (Xcorr in SEQUEST and e-Score in X!Tandem). Protein identities were assigned numerical priority scores (from “1” to “4”): “1” for high peptide confidence and multiple sequences; “2” for high peptide confidence; “3” for moderate peptide confidence and multiple sequences; or “4” for moderate peptide confidence. High peptide confidence corresponded to a highest confidence between 90%-100%, and moderate confidence to 75%-89%.

Protein quantification was carried out using a proprietary protein quantification algorithm (Higgs et al., 2005). Briefly, raw files were acquired from the LTQ and all extracted ion chromatograms (XIC) were aligned by retention time. For protein quantification, each aligned peak must match precursor ion, charge state, fragment ions (MS/MS data) and retention time (within a one minute window). After alignment, the area-under-the-curve (AUC) for each individually aligned peak from each sample was measured and compared for relative abundance. Peak intensities were log2 transformed before quantile normalization (Bolstad et al., 2003) to ensure that every sample has a peptide intensity histogram of the same scale, location, and shape. Normalization removed trends introduced by sample handling, sample preparation, total protein differences and changes in instrument sensitivity while running multiple samples. If multiple peptides had the same protein identification, then their quantile normalized log2 intensities were averaged to obtain log2 protein intensities.

### Example 7

### Performance of Statistical Analyses

Using JMP Genomics 4.0 (SAS Institute Inc., Cary, N.C.), overlaid kernel density estimates, univariate distribution results, correlation coefficients of pair wise sample comparisons, unsupervised principal components analysis (by Pearson product-moment correlation), and Ward hierarchal clustering of Pearson product-moment correlations were performed using log2 transformed data, as described (Mudge et al., 2008), herein incorporated in its entirety. Decomposition of principal components of variance, including patient demographics, past medical history, laboratory values, and clinical values, was performed to maximize sepsis-group-related components of variance and minimize residual variance. Guided by these analyses, ANOVA was performed between sepsis groups with five or 10% false discovery rate (FDR) correction and inclusion of substantive non-hypothesis components of variance as fixed effects. These included renal function, as determined by the estimated glomerular filtration rate (eGFR) using the four variable modification of diet in renal disease calculation, hemodialysis (HD), cirrhosis and liver disease, hepatitis, neoplastic disease, congenital disease, administration of exogenous immunosuppressants, drug abuse, metabolic dysfunction, respiratory dysfunction, serum glucose levels, and mean arterial pressure (MAP) in analyses as non-hypothesis components of certain metabolome and/or proteome datasets.

Predictive modeling was performed with JMP Genomics 4.0 using four approaches: (1) discriminant analysis, (2) general linear model selection (GLMS), (3) logistic regression, and (4) partition trees. Cross validation was performed using 50 iterations and 15% sample omission. Bayesian clinical factor analysis and BPFA-GPR was performed jointly for all metabolites and samples (Chen et al., manuscript submitted). To distinguish the effects of SIRS outcomes (non-infected SIRS+, sepsis survivors, and sepsis death) and relevant clinical factors on the metabolome, Bayesian clinical factor analysis [cⱼ=Byⱼ+A(sⱼ·zⱼ)+εⱼ] was used. The analysis correlates relevant metabolite patient changes to the clinical features to define the relevance of the clinical parameter. The formula defines B as the relationship between data and the clinical feature, while A defines random or undefined effects and ε accounts for random noise. The clinical features were further normalized to normal distribution with zero-mean and standard deviation. The significant features were then plotted on B-matrix as well as plotted as normalized energy of each clinical feature. In the BPFA-GPR analysis, for each metabolite there was an associated vector of responses, manifested by the response of each sample for that metabolite. Each such metabolite-dependent vector was represented as a linear combination of factors. A mixture model was employed, with each metabolite employing factors from one of the mixture components (clusters). The number of clusters needed to represent all metabolites was inferred via the Dirichlet process, metabolites were grouped into clusters and the latter were employed to build a sparse probit-regression classifier. Specifically, the classifier imposed that only a small subset of clusters be used (all metabolites from unused clusters were not employed in the classifier). However, when a cluster was used, multiple metabolites from the cluster were employed in the classifier. The use of a sparse set of metabolites mitigated over-training. Further, since multiple metabolites were used from the same cluster when that cluster is employed, correlated metabolites of relevance were retained.

### Experimental Results

### A. Review of Patient Demographics

Between 2005 and 2009 1152 individuals who presented at three urban, tertiary-care, emergency departments with suspected acute infection and at least two SIRS criteria (Bone et al., 1992) were enrolled in the Community Acquired Pneumonia and Sepsis Outcomes Diagnostic (CAPSOD) [NCT00258869]. Enrollee records were adjudicated for unambiguous sepsis classification. Sepsis was defined by either (1) the enrollment criteria plus the identification of causal microorganism, or (2) in cases of polymicrobial infections (i.e., infections in which organisms were identified only in cultures of non-sterile sites, or in unreplicated cultures yielding a likely commensal organism), by discharge diagnosis of sepsis. Patients meeting enrollment criteria, but with non-infectious discharge diagnosis and without confirmatory evidence of infection, were classified as non-infected SIRS-positive (NIS). The metabolomc and proteome were analyzed in plasma from five groups of approximately 30 patients selected from the 1152: (1) non-infected SIRS-positive (NIS), (2) uncomplicated sepsis (UCS, day 28 sepsis survivors who did not develop severe sepsis or septic shock within 3 days of enrollment (Bone et al., 1992; Bone et al., 1989; Balk 2000), (3) severe sepsis (SS; day 28 sepsis survivors who did not develop septic shock within 3 days but did develop severe sepsis, as defined by acute renal, respiratory, hematologic or hepatic dysfunction, Table 2 (Bone et al., 1992; Bone et al., 1989; Balk 2000), (4) septic shock (SShock; day 28 sepsis survivors who developed shock within 3 days), and (5) sepsis deaths (SD; within 28 days of enrollment). Sepsis patients were also selected to maximize those with known microbiologic etiology particularly Escherichia coli, Staphylococcus aureus, and Streptococcus pneumoniae. Groups were matched for age, race, sex and enrollment site (Table 3).

At enrollment (t₀), the APACHE II score, age, mean arterial pressure (MAP), temperature, serum sodium, and hematocrit differed significantly between these groups (Table 4). At t₀, the APACHE II score was elevated, as expected, in SS, SShock, and SD (Knaus et al., 1985; Russel 2008). At t₀, pyrexia was pronounced in patients who had UCS or SS (but not SShock or SD), which was consistent with the clinical analysis of 730 patients (Glickman et al., 2010).

TABLE 4
CLINICAL AND LABORATORY VALUES FOR PATIENTS (MEAN ± SE)

| | Total Number of Patients | | | | |
|---|---|---|---|---|---|
| | | 27 | 25 | 38 | 31 |
| | 29 | Uncomplicated | Severe | Septic | Sepsis |
| | Mimics | Sepsis | Sepsis | Shock | Death |
| Patient History | | | | | |
| Heart Failure | 3.4% | 14.8% | 28.0% | 23.7% | 25.8% |
| Liver Failure | 6.9% | 0.0% | 8.0% | 5.3% | 19.4% |
| Diabetes Melitus | 34.5% | 25.9% | 24.0% | 34.2% | 41.9% |
| Neoplastic Disease | 24.1% | 3.7% | 8.0% | 5.3% | 22.6% |
| Chronic Lung Disease | 37.9% | 14.8% | 36.0% | 28.9% | 25.8% |
| Renal Failure | 20.7% | 14.8% | 40.0% | 15.9% | 22.6% |
| Hemodialysis | 13.8% | 14.8% | 32.0% | 13.2% | 9.7% |
| Late HIV | 0.0% | 0.0% | 8.0% | 0.0% | 3.2% |
| Immunosuppresants | 3.4% | 0.0% | 12.0% | 5.3% | 6.5% |
| Smoker | 17.2% | 37.0% | 28.0% | 23.7% | 25.8% |
| Alcohol Use | 17.2% | 11.1% | 16.0% | 18.4% | 12.9% |
| Drug Abuse | 10.3% | 29.6% | 28.0% | 15.8% | 12.9% |
| Clinical Variables | | | | | |
| Age (years)* | 65.8 ± 13.6 | 53.8 ± 15.4 | 55.0 ± 16.8 | 58.6 ± 18.5 | 68.8 ± 16.7 |
| Heart Rate (per min) | 107.1 ± 20.1 | 110.0 ± 19.9 | 123.0 ± 20.3 | 115.7 ± 24.9 | 114.2 ± 25.3 |
| Respiratory Rate | 25.2 ± 6.4 | 23.1 ± 5.3 | 27.4 ± 9.7 | 24.7 ± 6.4 | 28.9 ± 10.4 |
| (per min) | | | | | |
| Temperature (° C.)* | 36.8 ± 1.1 | 38.2 ± 1.8 | 38.5 ± 1.0 | 37.7 ± 2.0 | 37.4 ± 1.7 |
| MAP (mmHG)* | 89.3 ± 20.1 | 90.4 ± 13.6 | 82.5 ± 15.8 | 71.3 ± 15.8 | 69.0 ± 13.5 |
| SOFA | 4.4 ± 2.9 | 3.7 ± 1.2 | 4.7 ± 2.2 | 5.7 ± 3.1 | 7.0 ± 3.6 |
| APACHE II* | 17.6 ± 7.2 | 11.1 ± 5.9 | 18.6 ± 5.6 | 19.1 ± 7.1 | 23.5 ± 9.0 |
| Lab Values | | | | | |
| Sodium (mMol/L)* | 136.9 ± 4.4 | 137.2 ± 3.2 | 134.2 ± 5.9 | 136.7 ± 5.1 | 141.6 ± 10.6 |
| Potassium (mMol/L) | 4.7 ± 1.3 | 3.9 ± 0.8 | 4.5 ± 1.2 | 4.3 ± 1.0 | 4.3 ± 1.1 |
| Creatinine (mg/100 mL) | 2.7 ± 3.7 | 2.6 ± 3.5 | 3.9 ± 4.3 | 2.7 ± 2.8 | 2.7 ± 2.9 |
| BUN (mg/dL) | 34.8 ± 27.2 | 20.6 ± 17.6 | 43.1 ± 41.9 | 31.0 ± 22.4 | 47.5 ± 40.0 |
| Glucose (mg/dL) | 151.0 ± 96.1 | 142.1 ± 82.0 | 190.5 ± 192.3 | 157.3 ± 107.1 | 164.3 ± 157.9 |
| Hematocrit (%)* | 34.8 ± 6.6 | 38.4 ± 5.2 | 37.5 ± 4.8 | 33.9 ± 7.7 | 30.6 ± 7.4 |
| WBC (1000 s/mm³) | 10.8 ± 4.2 | 12.9 ± 4.4 | 15.1 ± 8.7 | 16.4 ± 8.5 | 18.6 ± 18.3 |
| Platelet (10³/mm³) | 275.5 ± 98.9 | 240.3 ± 77.8 | 214.9 ± 163.5 | 235.6 ± 126.0 | 232.0 ± 151 |
| eGFR (mL/min) | 65.7 ± 53.0 | 75.5 ± 24.6 | 77.7 ± 48.6 | 75.7 ± 77.5 | 55.8 ± 40.3 |

*Significant difference between groups by ANOVA with Bonferroni correction (p ≦ 0.0031)

### B. Overview of the Metabolome

Of approximately 4,413 biochemicals detectable in human biofluids (Wishart et al., 2009), 439 were measured in venous plasma from the 150 discovery patients using label-free, combined liquid and gas chromatography and MS at t₀ or t₂₄, of which 332 were measured at both times. A total of 215 and 224 biochemicals, at t₀ and t₂₄, respectively, were annotated human metabolites (FIG. 14).

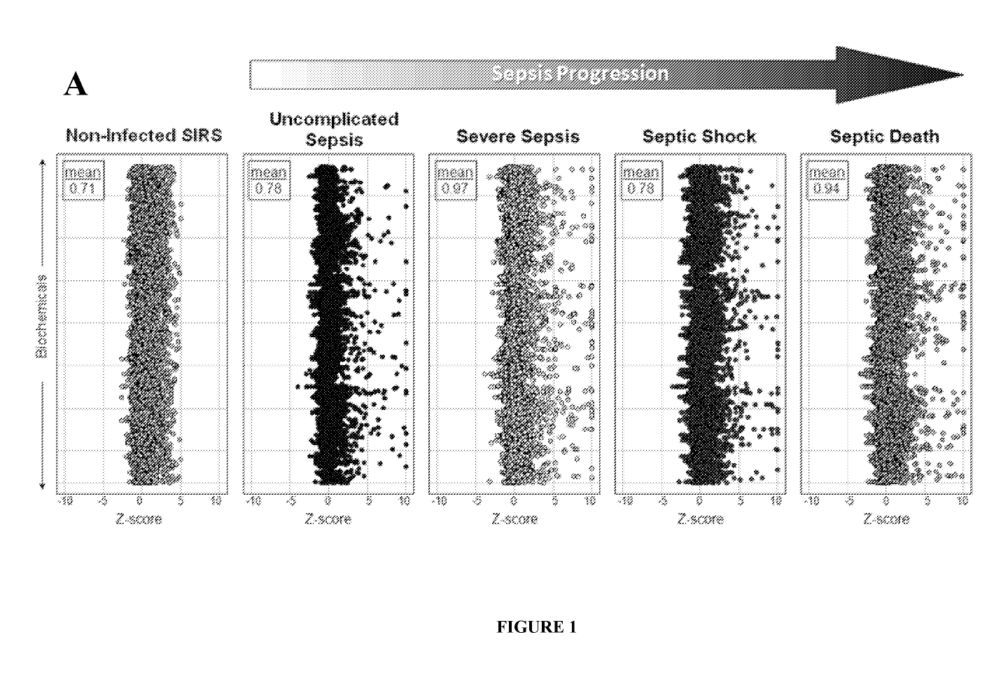

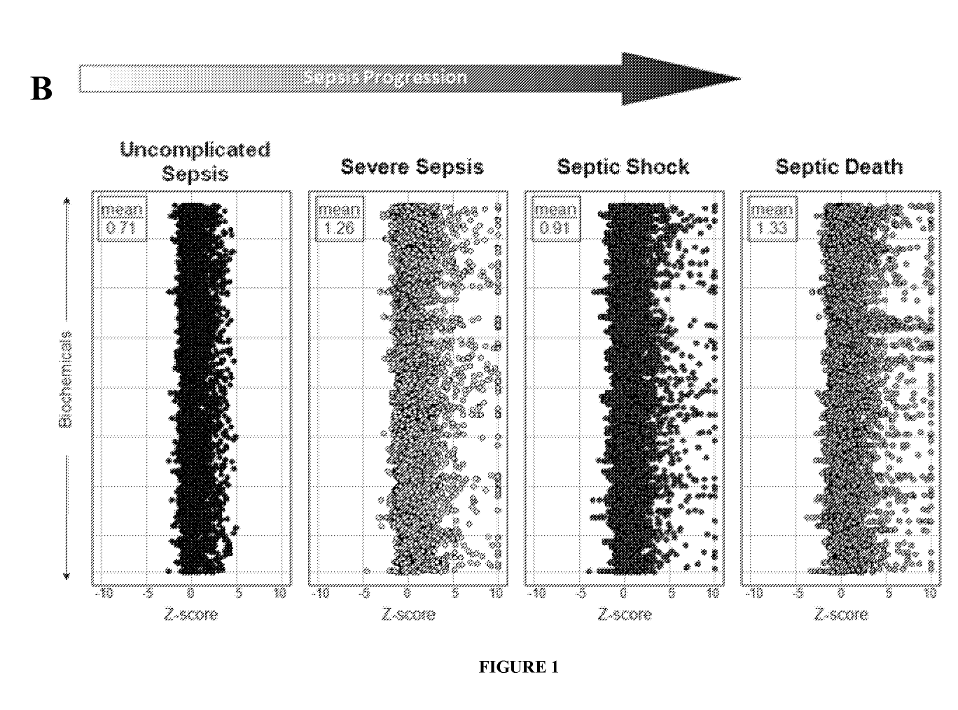

At t₀, the platform and overall variability were 10% and 17%, respectively. When measured by clinical chemistry and log2-transformed mass spectrometry, the serum and plasma creatinine (n=149), lactate (n=115), and glucose levels (n=149) at t₀ correlated well (r² 0.90, 0.58, 0.56, respectively). At t₀, no samples were outliers (as assessed by Mahalanobis differences). Overlaid kernel density estimates indicated approximately normal distribution of log-transformed metabolite values. Healthy controls typically have normally distributed metabolite Z-scores, with most metabolites distributed within two standard deviations of the mean and centered at the origin. At t₀, the Z-scores of metabolites in NIS were right-skewed, with most values falling within five standard deviations of the mean (FIG. 1A). This reflects a wide spectrum of illness severity (no complication (n=8); organ dysfunction within 72 hours (n=12); shock within 72 hours (n=6); death within 28 days (n=3)). Relative to NIS, at t₀, the Z-scores of metabolites of other groups were increased particularly those in SS and SD (FIG. 1A). FIG. 1A shows the Z-score scatter plots for biochemicals observed in human plasma obtained from mimic patients or UCS patients, with severe sepsis (by day 3 post-enrollment), septic shock (by day 3), or septic death (by day 28). Groups were defined by the most severe stage of sepsis attained by the individual. The zero point on the X-axis represents the mean of the control group (NIS). Each data point is expressed as the number of standard deviations from the mean of the control group. The Y-axis represents the biochemicals, with all data for any single biochemical represented on the same horizontal line. Patient data are also expressed in standard deviations from the control mean, thus revealing their change relative to control. The mean values shown represent the mScore, which is an average of the absolute values of Z-scores for all metabolites, calculated using non-truncated, non-imputed values (in NIS, mean=0.71; in UCS, mean=0.78; in severe sepsis, mean=0.97; in septic shock, mean=0.78; in septic death, mean=0.94). FIG. 1B shows the Z-score scatter plots for sepsis patients, using the UCS group as the control. Similar changes in the distribution of Z-scores were observed when UCS patients served as the reference group (UCS, mean=0.71; severe sepsis, mean=1.26; septic shock, mean=0.91; and septic death, mean=1.33). These data indicate sepsis to alter plasma metabolites at t₀ in a manner that varies according to stage of sepsis progression, reference characteristic.

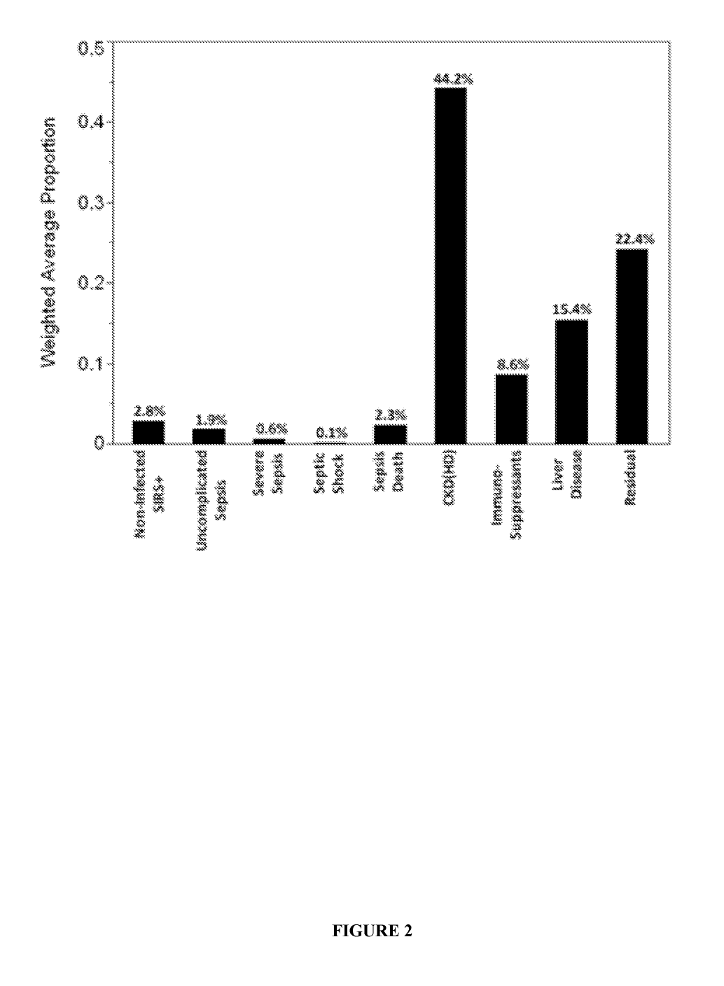

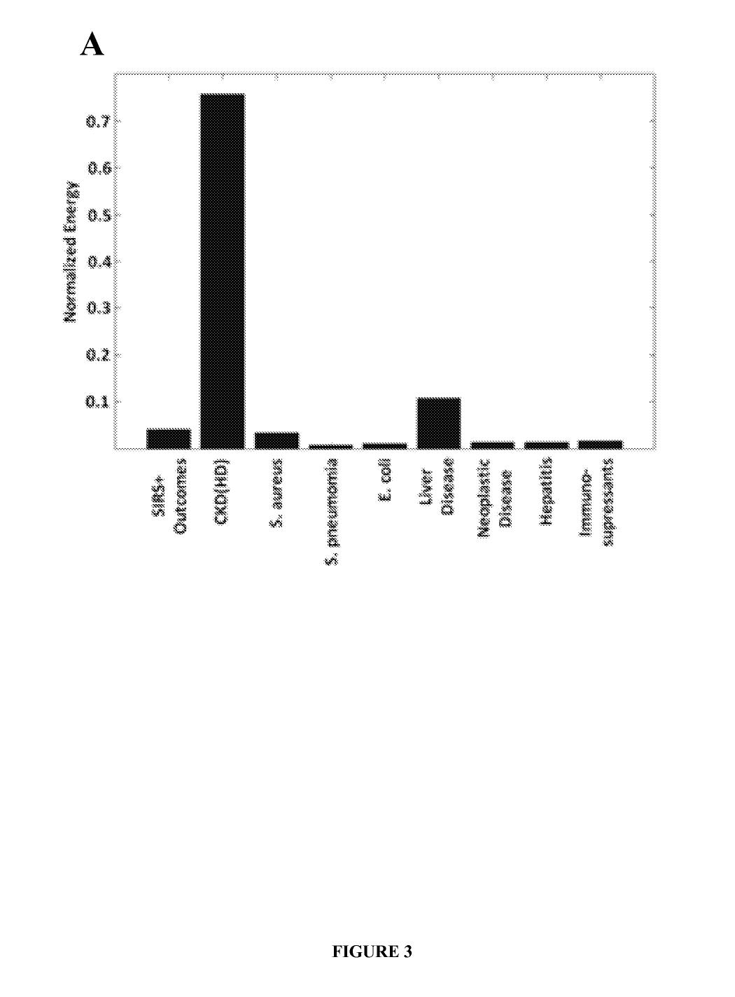

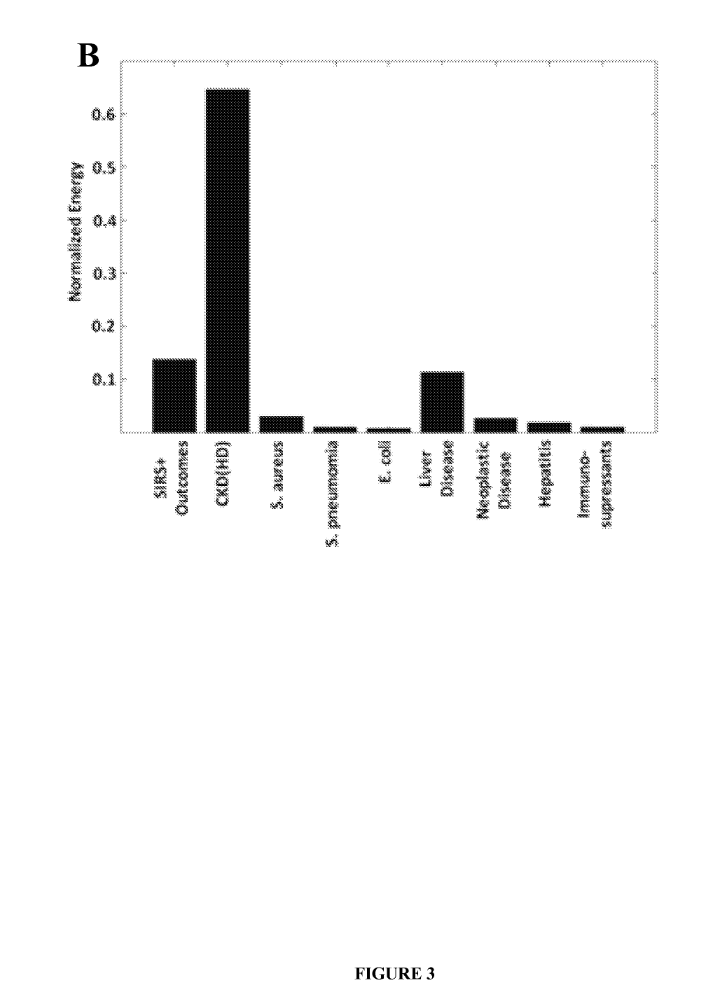

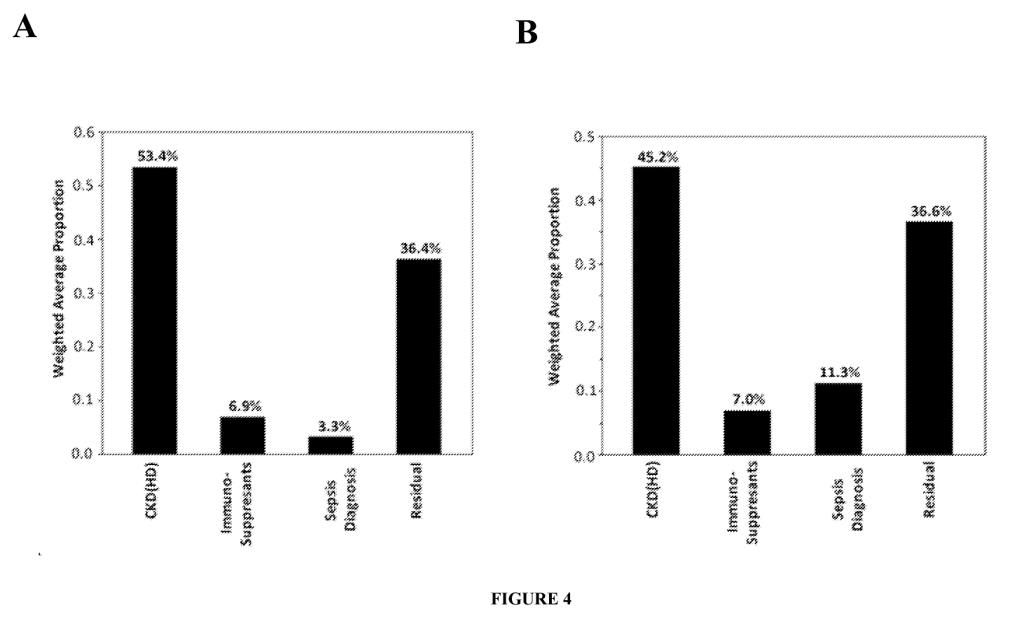

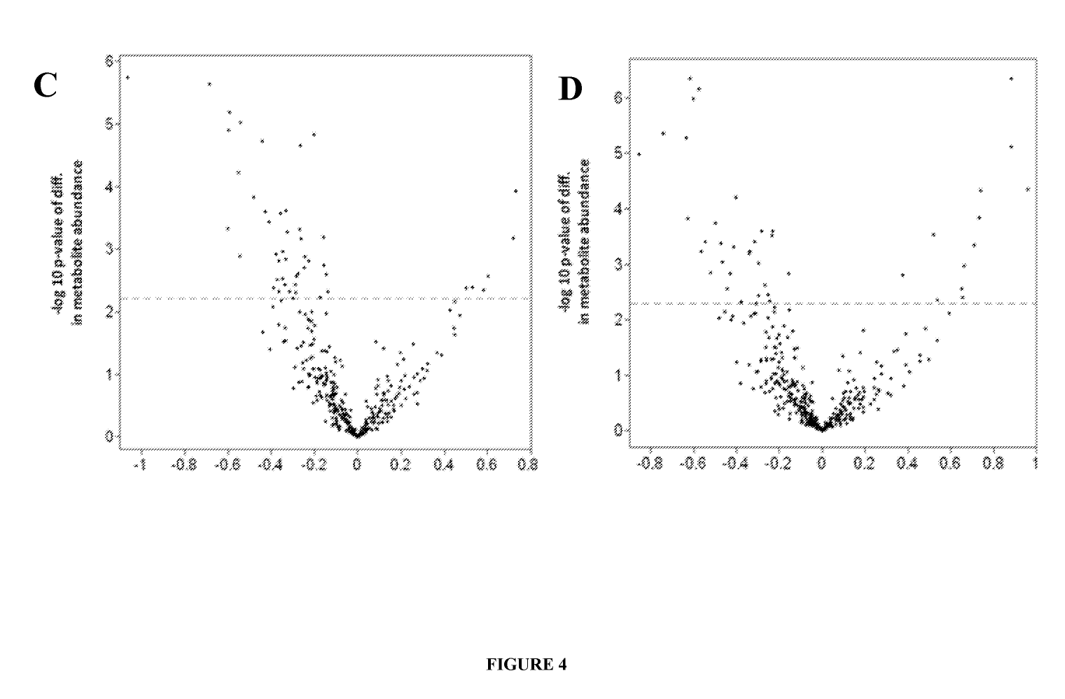

Principal component analysis and Bayesian factor analysis (Chen et al. submitted) and normalized energy plots showed renal function, liver disease and sepsis group membership to largely define, in descending order, variation in the plasma metabolome (FIG. 2-3). The magnitude of sepsis group effects, but not renal function or liver disease, was greater at t₂₄ than at t₀ (FIGS. 3-5), indicating metabolic perturbations associated with sepsis to increase in the day after seeking medical attention. At t₀, eGFR was as follows: 69.3±55.5 (Non-infected SIRS positive), 71.2±56.9 (Sepsis Survivors), and 60.2±40.2 (Sepsis Death). At t₂₄, eGFR was as follows: 68.1±48.9 (Non-infected SIRS positive), 75.9±55.6 (Sepsis Survivors), and 59.6±31.6 (Sepsis Death). The p-value was as follows: 0.83 (Non-infected SIRS positive), 0.16 (Sepsis Survivors), and 1 (Sepsis Death

Since the average and median time to death were 8 and 10.7 days, respectively, this increase was not due to imminent death and likely reflects enrollment at an early stage in sepsis progression. The unsupervised PCA of sepsis group membership and at t₀ demonstrated that for sepsis groups that CKD(HD), liver disease, and immunosuppressant therapy, were substantive, non-hypothesis components of variance. CKD(HD): estimated glomerular filtration rate (eGFR), per Chronic Kidney Disease (CKD) criteria, and hemodialysis. The renal function in log-transformed plasma metabolites demonstrated the following: CKD1/2 (GFR>74 mL/min, n=44), CKD3 (eGFR 32-74 mL/min, n=56), CKD4/5 (eGRF 0-31 mL/min, n=25), and hemodialysis (HD, n=24).

FIG. 3 shows the normalized energies of sepsis group membership (SIRS+Outcomes), renal category (CKD(HD)) and other clinical parameters in log-transformed plasma metabolites at t₀ and t₂₄. Sepsis group membership (SIRS+Outcomes) was defined as non-infected SIRS-positive, sepsis survival and sepsis death. Renal function was defined as eGFR>74 mL/min=0; 32-74 mL/min=1; <31 mL/min=2; and hemodialysis=3. Clinical parameters were fit to a normal distribution with mean of zero and standard deviation of one. Bayesian regression [cⱼ=Byⱼ+A(sⱼ·zⱼ)+cⱼ where B is the relationship between metabolite values and the clinical parameter, A is random or undefined effects and ε is random noise] of metabolite values and clinical parameters defined the relevance of the latter. CKD(HD), liver disease and SIRS+Outcomes largely define changes in the plasma metabolome at t₀ in descending order. Normalized energy of sepsis group membership (SIRS+Outcomes) increased from 0.06 at t₀ to 0.14 at t₂₄.

Differences between sepsis groups were sought by analysis of variance (ANOVA) and Bayesian factor analysis. Non-hypothesis-related effects were minimized, in the former, by inclusion of renal function and liver disease as fixed effects and, in the latter, by separation of renal and sepsis group effects. In death comparisons this was too strict since acute renal dysfunction partly co-segregated with sepsis death. (Table 5)

TABLE 5
PARTIAL OVERLAP OF EGFR GROUP AND
SEPSIS GROUP MEMBERSHIP

| Estimated Glomerular | | Non-infected | Uncomplicated | Severe | | Sepsis |
|---|---|---|---|---|---|---|
| Filtration Rate | N | SIRS-Positive | Sepsis | Sepsis | Septic Shock | Death |
| >74 ml/min | 45 | 13.3% | 26.7% | 15.6% | 24.4% | 20% |
| 32-74 ml/min | 54 | 27.8% | 20.4% | 14.8% | 16.7% | 20% |
| 0-31 ml/min | 28 | 17.9% | 3.6% | 10.7% | 35.7% | 32.1% |
| Chronic hemodialysis | 22 | 13.6% | 13.6% | 36.4% | 27.3% | 9.1% |

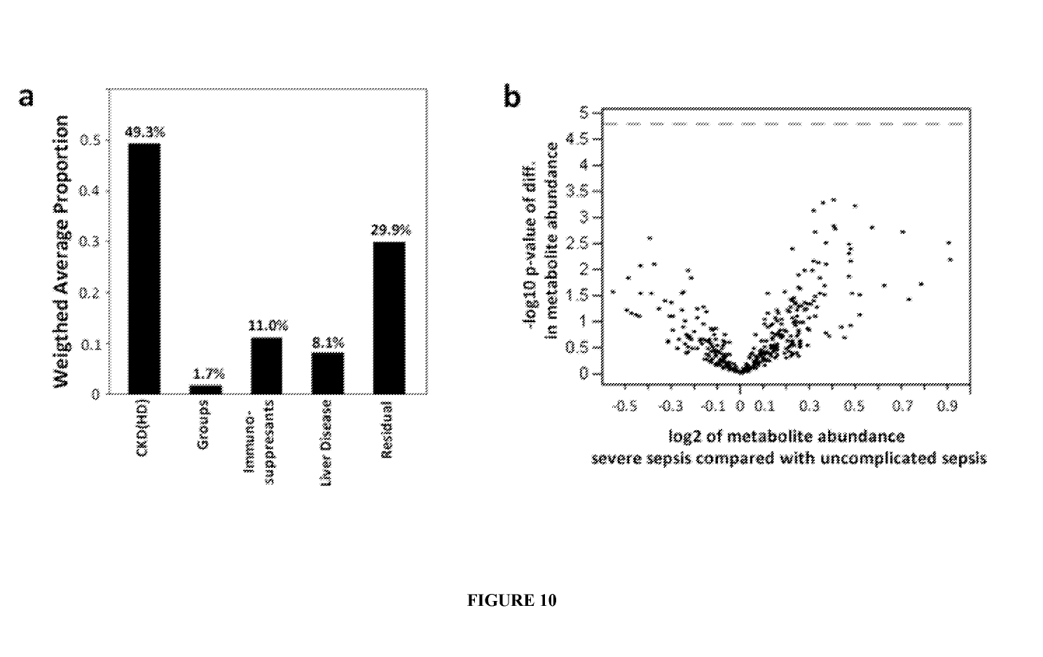

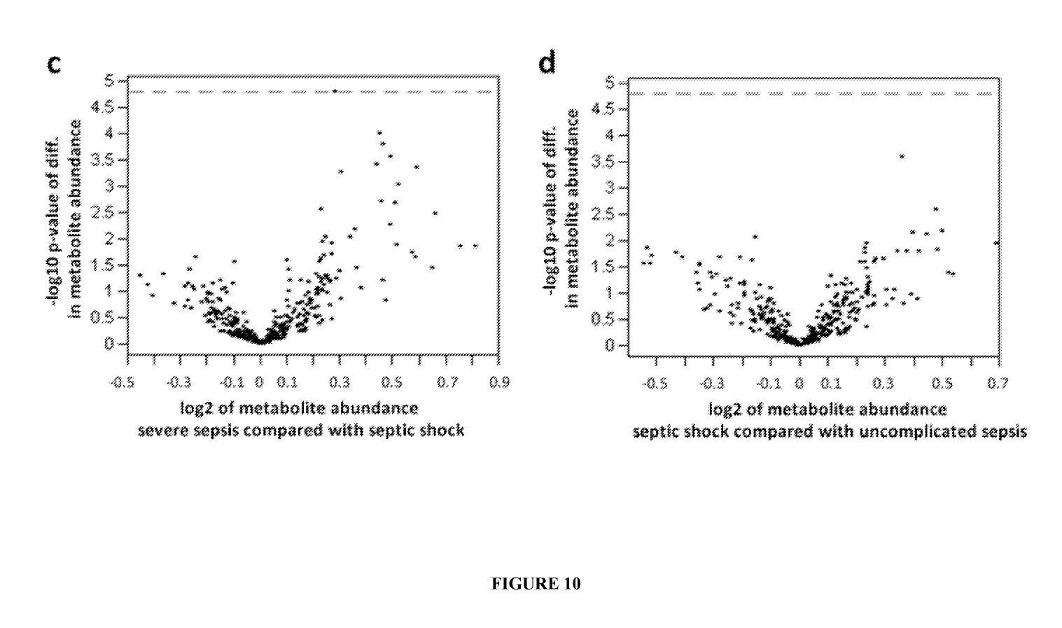

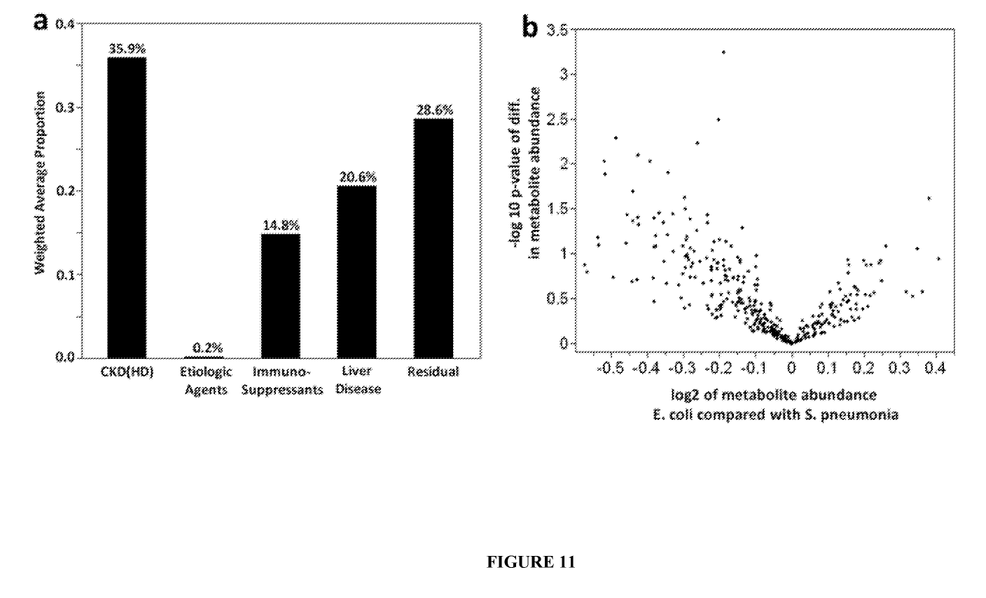

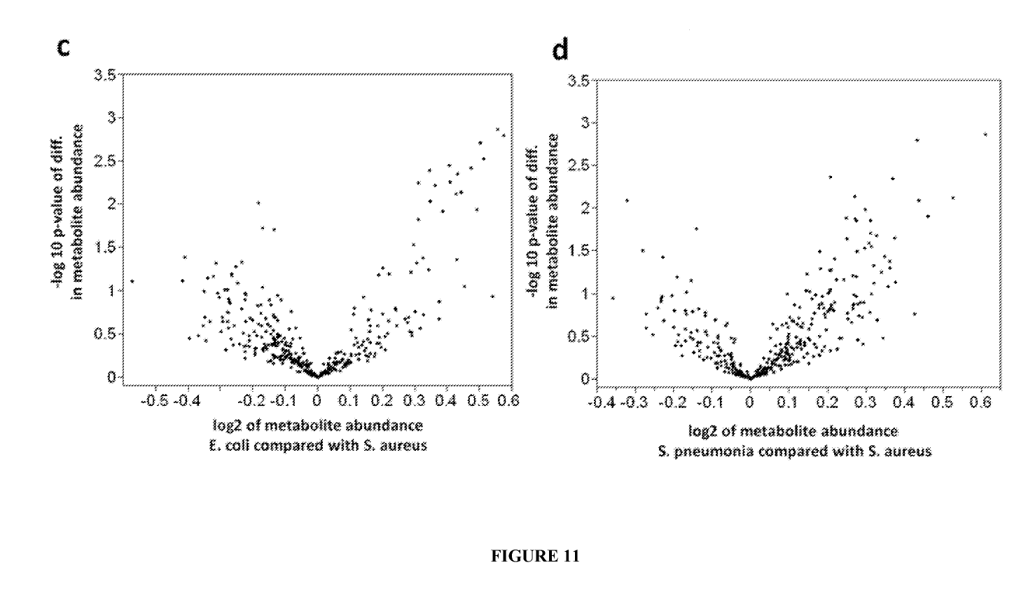

There were no significant metabolic differences among sepsis survivor groups (uncomplicated sepsis, severe sepsis and septic shock), nor unique to sepsis due to S. pneumoniae, S. aureus and E. coli (FIG. 10 and FIG. 11). These data suggest sepsis survivors to represent a metabolic continuum at presentation, irrespective of imminent clinical course. Similarly, they suggest that the initial host metabolic response to bacterial sepsis is largely unaltered by etiology.

FIG. 10 shows the principal components of plasma metabolomic variance (with Pearson product-moment correlation) of sepsis survivor groups at to (A) and volcano plots of FDR-corrected (5%) ANOVAs with non-hypothesis components of variance as fixed effects in (B)-(D). No significant differences were found between survivors with uncomplicated sepsis and severe sepsis (B), severe sepsis and septic shock (C), or uncomplicated sepsis and septic shock (D). Significantly altered metabolite levels would be indicated by dots above the red line. The variation explicable by these groups (1.7%) is too small to detect meaningful changes in host plasma mebolite values.

FIG. 11 shows principal components of plasma metabolomic variation associated with etiologic agent in sepsis at t₀ and volcano plots of weighted ANOVAs. In FIG. 11A, the principal components of variance decomposition (with Pearson product-moment correlation) for etiologic agents and clinical parameters are shown. Volcano plots of FDR-corrected (5%) ANOVAs (with non-hypothesis components of variance as fixed effects) indicate no significant differences between host metabolic response to bacteremia with E. coli (n=16) and S. pneumoniae (n=31) in (B), with E. coli and S. aureus (n=27) in (C), and S. pneumoniae and S. aureus in (D). The variation explicable by these groups (0.2%) is too small to detect meaningful changes in host plasma mebolite values.

### (I). Metabolic Differentiation of Sepsis Survivors and NIS at T₀ & T₂₄

Sepsis survivors differed from non-infected controls in plasma levels of 49 and 42 metabolites at t₀ and t₂₄, respectively, which—be reference characteristics (stratified ANOVA, FDR 5%; FIG. 14). Sixty of 63 metabolites detected at both times had concordant changes, suggesting a persistent metabolic response to sepsis (FIGS. 6-9, 12; Table 6) reference characteristic. Sepsis survivors exhibited significant decreases in many carnitine and glycerolphosphocholine esters, amino acids and their catabolites, complex sugars, citrate, malate, glycerol, glycerate and phosphate (ANOVA; FIGS. 6-9; Table 6) reference characteristic. Several free fatty acids and steroids were increased, reference characteristic. Lactate and ketones were unchanged, reference characteristic. In addition, sepsis survivors exhibited an average elevation in temperature of 1.3° C., reference characteristic. Cumulatively, these alterations attest to elevated mobilization and aerobic metabolism of lipid, protein and sugar substrates in sepsis survivors, reference characteristic (Giovannini et al. 1983; Bernard et al. 2001; Brealey et al. 2002; Fukumoto et al. 2003; Herzum and Renz 2008; Pierrakos and Vincent 2010).

TABLE 6
DIRECTION OF CHANGE OF SIGNIFICANT PLASMA
METABOLITE DIFFERENCES

| | Sepsis Diagnosis | | Sepsis Outcome | |
|---|---|---|---|---|
| | Increased¹ | Decreased | Increased² | Decreased |
| Discovery Set t₀ | 7 | 42 | 61 | 15 |
| Discovery Set t₂₄ | 12 | 30 | 112 | 16 |
| Replication Set t₀ | | | 12 | 6 |
| Replication Set t₂₄ | | | 13 | 7 |

In Table 6, sepsis diagnosis shows the comparison of sepsis survivors with non-infected SIRS-positive patients and sepsis outcome shows the comparison of sepsis survivors and deaths. Significant differences reflect weighted ANOVAs with 5% FDR (t₀ and t₂₄ in the discovery set), 25% FDR (to in the replication set) or 15% FDR (t₂₄ in the replication set).

### (II) Metabolic Differentiation of Sepsis Survivors & Sepsis Deaths

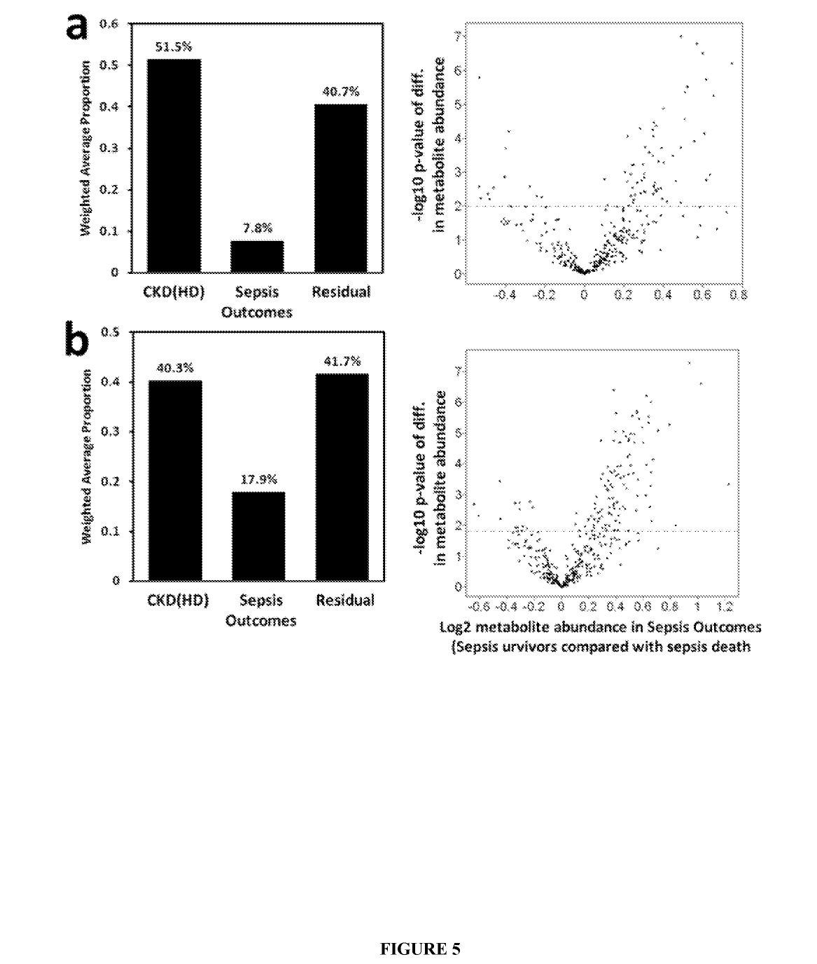

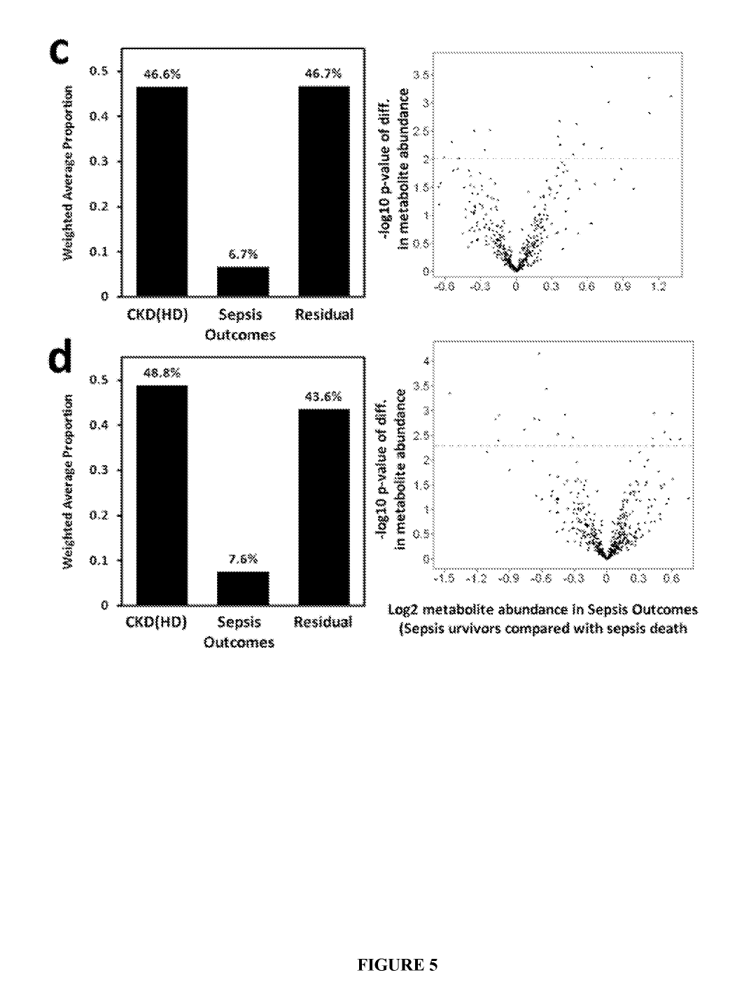

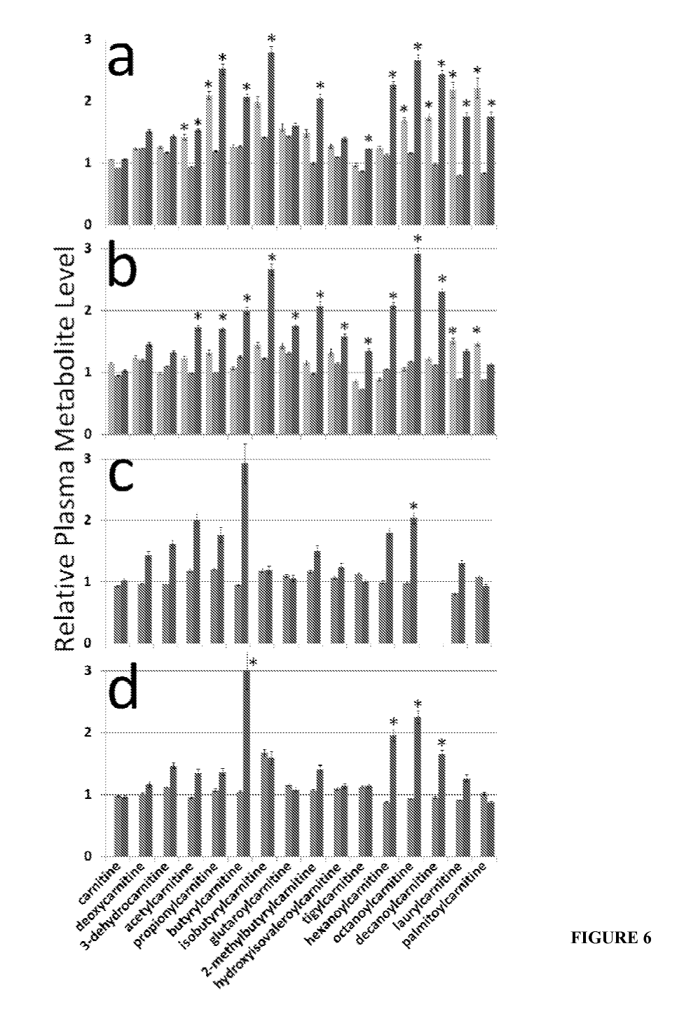

Sepsis survivors and day 28 sepsis deaths differed in levels of 76 and 128 plasma metabolites at t₀ and t₂₄, respectively (stratified ANOVA, FDR 5%; FIGS. 5-9, 14; Table 6). Fifty five of these were significant and 84 had concordant changes at t₀ and t₂₄, indicating sustained metabolic differentiation of sepsis survivors and future deaths. Strikingly, patients who would die exhibited antithetical metabolic changes from survivors that were present upon seeking medical attention and burgeoned with time: Thus, camitine esters, amino acid catabolites, modified nucleosides, citrate, malate, pyruvate, lactate and fatty acids were significantly elevated in sepsis patients who would die both by ANOVA, reference characteristic. Lactate elevation is a widely-used marker of adverse outcome in sepsis. Interestingly, anabolic steroids were decreased in sepsis deaths, suggesting an antecedent to some changes in intermediary metabolism, reference characteristic. Exceptions were acyl-GPCs, which showed additional decreases in sepsis deaths, in agreement with previous studies (Drobnik et al., 2003; Lissauer et al., 2007).

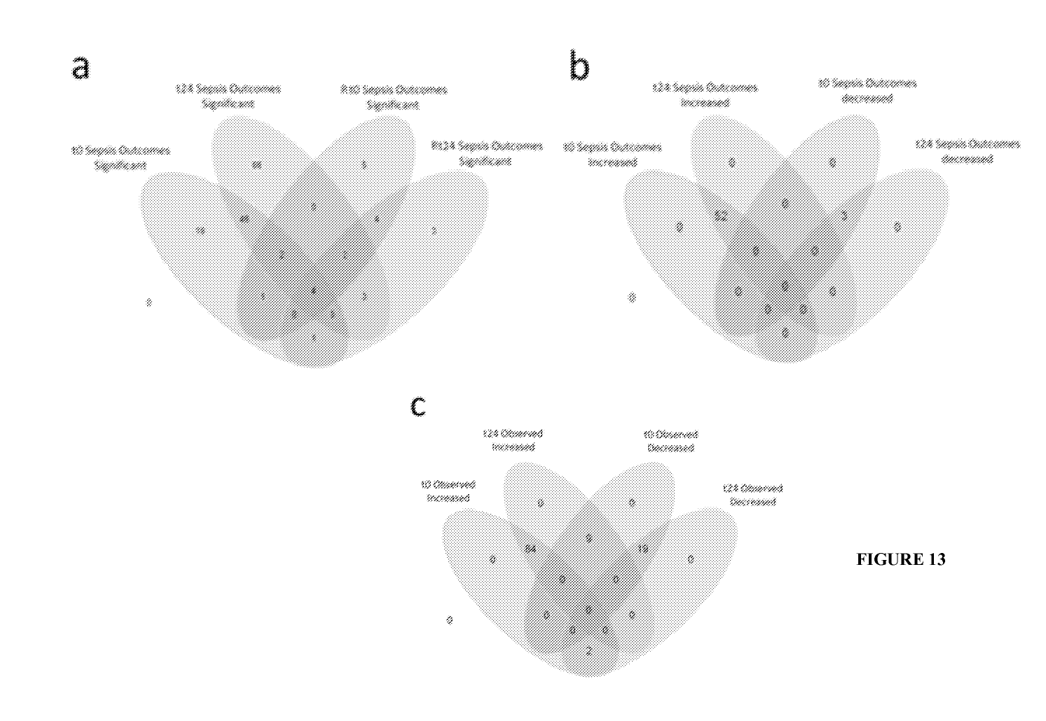

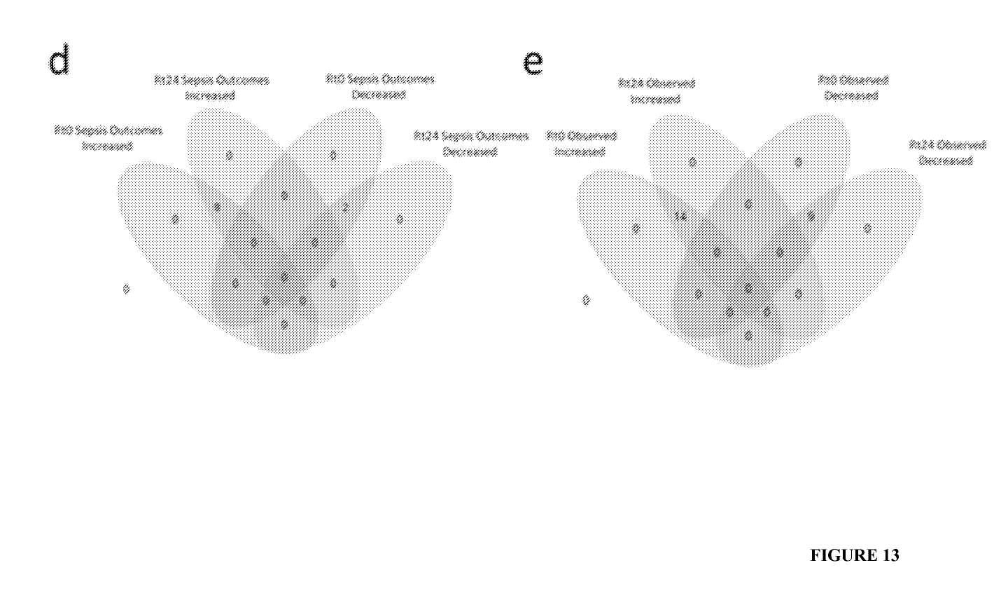

FIG. 13 shows Venn diagrams of significant differences in plasma metabolite levels between sepsis survivors and deaths at t₀ and t₂₄ in the discovery and replication (R) cohorts (A), concordance of direction of change of significantly different metabolites (B and D), and concordance of direction of change of metabolites with significant differences at one of the time points (C and E). Significant differences reflect weighted ANOVAs with 5% FDR (t₀ and t₂₄ in the discovery set), 25% FDR (t₀ in the replication set) or 15% FDR (t₂₄ in the replication set).

FIG. 5 shows plasma metabolite changes in sepsis outcomes (survival or death) in the discovery cohort at t₀ (A) and t₂₄ (B), and in the replication cohort at t₀ (C) and t₂₄ (D). In FIG. 5A-5D, the left panel shows variance decomposition with Pearson correlation of known parameters. In FIG. 5A-5D, the right panel shows volcano plots illustrating significant metabolite differences (i.e., the points above the line) by ANOVA with non-hypothesis variance parameters as fixed effects. At t₀ and t₂₄, FDR=5%. At t₀, replication=25% at t₂₄, replication=15%;

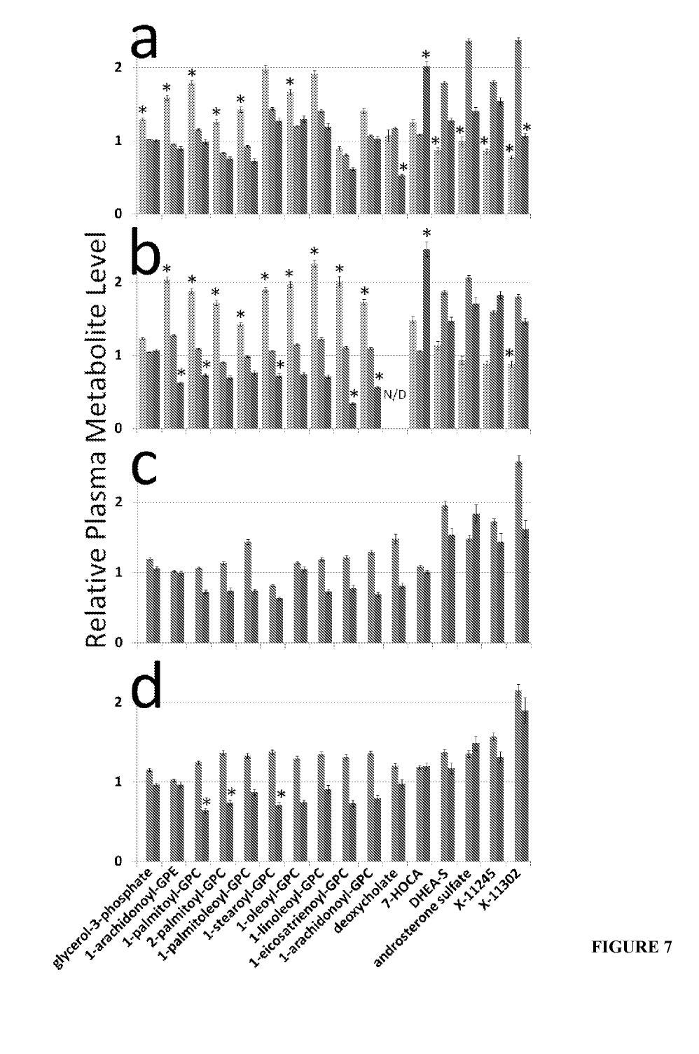

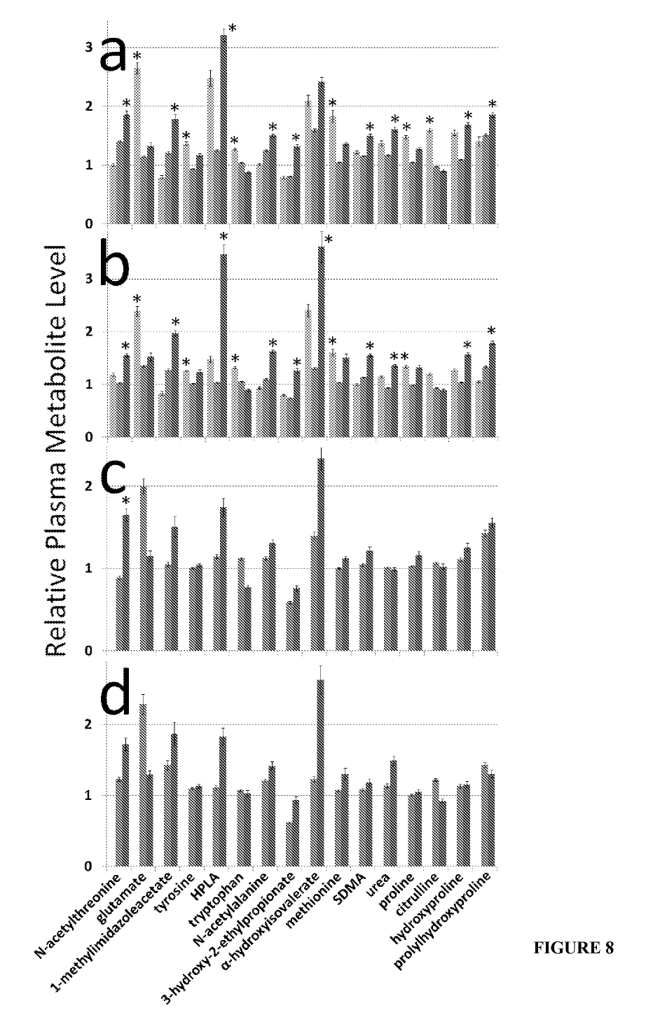

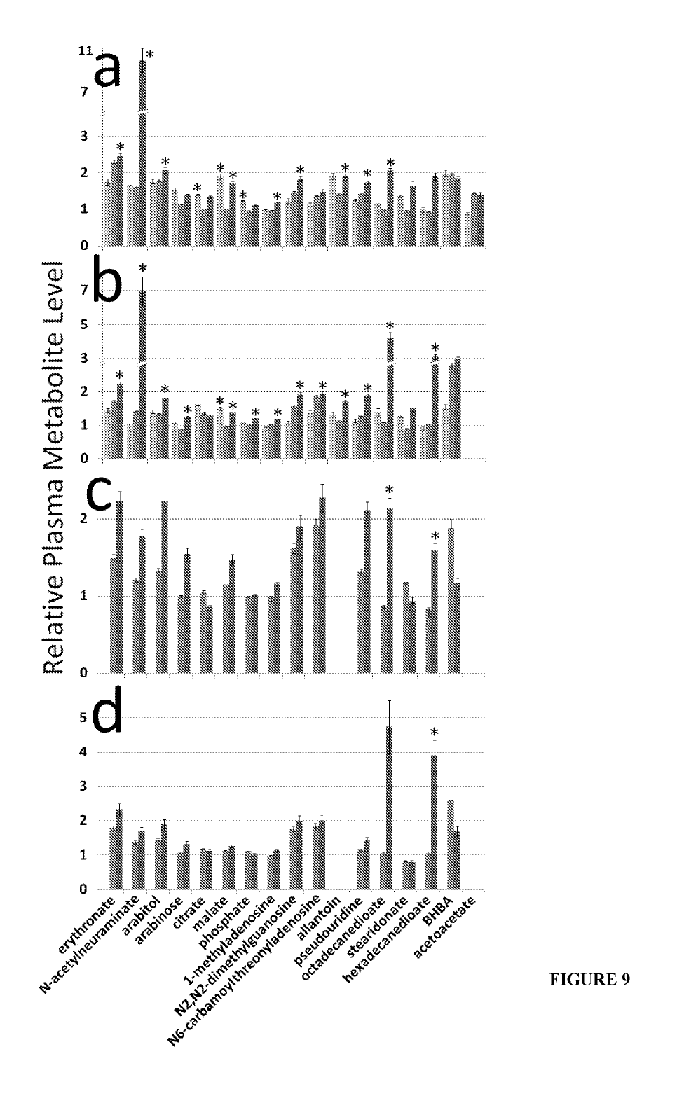

FIGS. 6-9 shows plasma metabolite levels for 16 (FIG. 6), 16 (FIG. 7), 16 (FIG. 8), and 17 (FIG. 9) metabolites in sepsis at t₀ (A), t₂₄ (B) and in replication patients at t₀ (C) and t₂₄ (D). Y-axis displays average, scaled, plasma metabolite concentrations. Error bars are SEM. In (A) and (B), from left to right for each metabolite, the three columns respectively represent controls (non-infected, SIRS positive), sepsis survivors, and sepsis deaths. In (C) and (D), from left to right for each metabolite, the columns respectively represent sepsis survivors and sepsis death. Asterisks indicate significant differences from sepsis survivors by weighted ANOVA with 5% FDR in (A) and (B), 25% FDR in (C), or 15% FDR in (D). All but carnitine, deoxycarnitine, 3-dehydrocarnitine, 3-dehydrocarntine, steridonate, 3-hybrodroxybutyrate (BHBA) and acetoacetate were significant; the latter were relevant negatives. Abbreviations: Glycerophosphethanolamine (-GPE), glycerolphosphocholine (-GPC), 7-α-hydroxy-3-oxo-4-cholestenoate (7-HOCA), dehydroepiandrosterone sulfate (DHEA-S), 3-[4-hydroxyphenyl]lactate (HPLA), symmetric dimethylarginine (SDMA), unannotated disulfated steroids (X-11245 and X-11301).

Replication of these findings was sought by MS-based metabolic profiling of plasma from 52 additional, sepsis survivors and deaths at t₀ and t₂₄. The replication set exhibited smaller effect sizes and longitudinal increases in differences between sepsis outcomes than the discovery set, consistent with much longer average and median times to death (15.7 and 18.5 days, respectively; FIG. 5). As a result, fewer significant differences were detected between sepsis survivors and deaths (18 at t₀ and 20 at t₂₄; FIGS. 6, 12, 14, Table 6). However, 10 of these were concordant and significant at both time points and all of the major metabolic perturbations in sepsis deaths were replicated (elevated carnitine esters, amino acid catabolites, modified nucleosides, citrate, malate and fatty acids, decreased anabolic steroids and GPC esters), reference characteristic. Together with absence of fever in sepsis deaths, these changes indicate a remarkable dichotomy of host metabolic response to sepsis long before death. Patients with sepsis who will die show similar mobilization of lipid, protein and sugar energetic substrates to those who survive, but fail to fully catabolize these aerobically, in stark contrast to sepsis survival.

Predictive (prognostic) modeling was performed using t₀ metabolite profiles, clinical variables and laboratory values using the etiologically homogenous SD (n=31) and sepsis survivor (n=90) groups as a training set. Of four predictive modeling methods employed, partition trees performed best (using predictor reductions of using K-means clustering, seven filtered predictors after t-test and FDR correction and forest analysis), yielding the classifier serum sodium, lactate, age, hematocrit, HPLA, 3-methoxytyrosine and the unannotated metabolite X-11302, reference characteristic. Accuracy (95.0%), and AUC (97.7%) were high, while RMSE (25.6%) was low; PPV was 98.9% and NPV was 83.9% (Table 7). Cross validation (50 iterations, 15% random holdouts) of the partition trees model yielded 76% accuracy, 74% AUC, and 40% RMSE. Validation t₂₄ comparisons yielded 93.5% accuracy, 97.5% AUC and 26.8% RMSE. PPV at t₂₄ was 100%, while NPV was 75.9% (Table 7). This is the prognostic performance anticipated in patients for whom infectious etiology has been established.

TABLE 7
PREDICTIVE MODELING FOR SEPSIS DEATH VS. SEPSIS SURVIVORS

| | | | | | Prediction | |
|---|---|---|---|---|---|---|
| Sepsis Death vs. Sepsis Survivors | | | | | Accuracy | |
| Comparison | Model | Accuracy | AUC | RMSE | PPV | NPV |
| | Sepsis Death (n = 31) vs. Sepsis Survivors (n = 90) | | | | | |
| Unsupervised | Partition | 0.9504 | 0.9771 | 0.2562 | 98.9 | 83.9 |
| | Trees | | | | | |
| Cross Validation | Partition | 0.76 ± 0.01 | 0.74 ± 0.02 | 0.40 ± 0.009 | * | * |
| | Trees | | | | | |
| | Sepsis Death (n = 29) vs. Sepsis Survivors (n = 78) | | | | | |
| t24 Validation | Partition | 0.9346 | 0.9748 | 0.2679 | 100.0 | 75.9 |
| | Trees | | | | | |

Predictive modeling was next performed using all survivors (sepsis and NIS survivors, n=116) and all 28-day deaths (sepsis and NIS deaths, n=34) as a training set. These groups represent the clinical complexity at time of presentation in the ED. Of the four predictive modeling methods, logistic regression and partition trees performed best. The logistic regression classifier contained six factors (mean arterial pressure, serum sodium, hematocrit, HPLA, piperine, and γ-tocopherol), reference characteristic, and was generated using predictor reductions of K-means clustering, less than 100 filtered predictors after t-test and FDR correction, and penalized reduction. The classifier generated by partition trees contained serum sodium, lactate, HPLA, acetylcarnitine, 3-methoxytyrosine and the unannotated metabolite X-11538, reference characteristic, (using K-means clustering, six filtered predictors after t-test and FDR correction and forest analysis). Performance of logistic regression and partition trees classifiers was clearly superior to APACHE II and SOFA, with 91.3% accuracy, 93.8% and 96.8% AUC, respectively, and 25.9% RMSE; PPV was 97.4% and NPV 70.6% for both models (Table 8). Cross validation (50 iterations, 15% random holdouts) of both models yielded 80.0% and 78% accuracy, 75% and 78% AUC and 40% and 38% RMSE respectively (Table 8). The models were validated using t₂₄ values. The partition trees classifier demonstrated highest t₂₄ accuracy at 92.4%, 96.5% AUC and 26.6% RMSE. PPV was 100% and NPV was 68.8% (Table 8). In comparison, the logistic regression classifier had 83.7% accuracy, 84.3% AUC, 34.9% RMSE and a PPV of 94.6% and NPV of 50%. Several of these factors selected are components of APACHE II and SOFA, including serum sodium, MAP, hematocrit and age.

TABLE 8
PREDICTIVE MODELING FOR SEPSIS DEATH VS. SEPSIS SURVIVORS

| | | | | | Prediction | |
|---|---|---|---|---|---|---|
| Death vs. Survivors | | | | | Accuracy | |
| Comparison | Model | Accuracy | AUC | RMSE | PPV | NPV |
| | SD (n = 34) vs. SS (n = 116) | | | | | |
| Unstructured | Logistic | 0.9133 | 0.9381 | 0.2585 | 97.4 | 70.6 |
| | Regression | | | | | |
| | Partition Trees | 0.9133 | 0.9675 | 0.2592 | 97.4 | 70.6 |
| Cross | Logistic | 0.8 ± 0.011 | 0.75 ± 0.18 | 0.4 ± 0.005 | * | * |
| Validation | Regression | | | | | |
| | Partition Trees | 0.78 ± 0.011 | 0.78 ± 0.18 | 0.38 ± 0.005 | * | * |
| | SD (n = 31) vs. SS (n = 100) | | | | | |
| t₂₄ validation | Logistic | 0.8374 | 0.8430 | 0.3494 | 94.6 | 50.0 |
| | Regression | | | | | |
| | Partition Trees | 0.9242 | 0.9650 | 0.2657 | 100.0 | 68.8 |

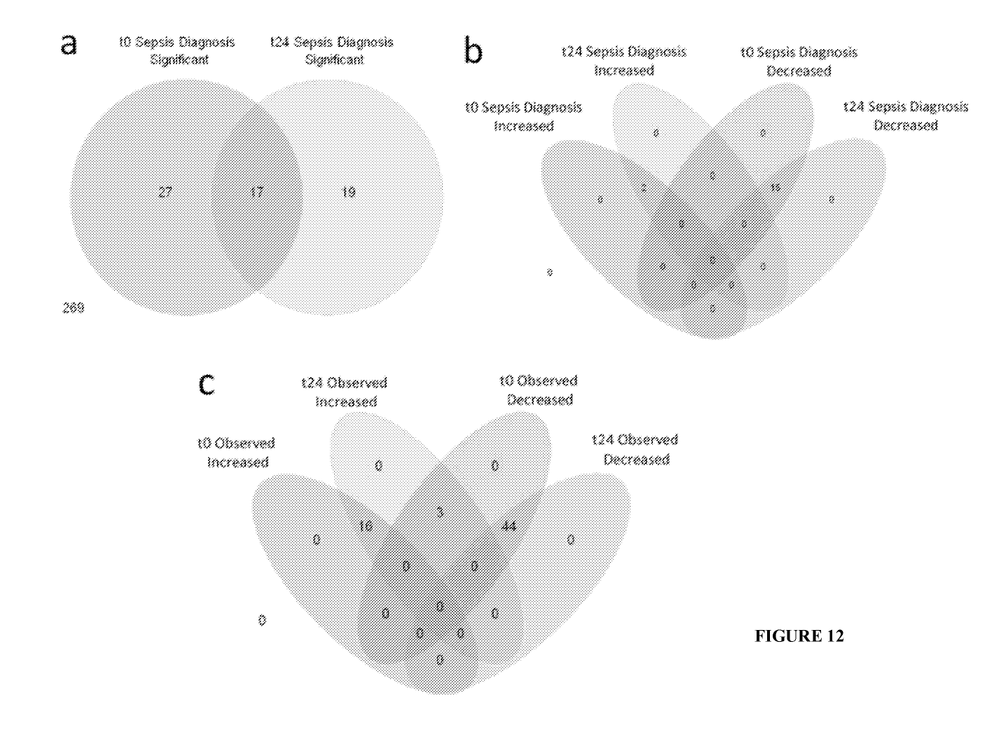

FIG. 12 shows Venn diagrams of significant differences (weighted ANOVA, 5% FDR) in plasma metabolite levels between non-infected patients (with SIRS) and sepsis survivors at t₀ and t₂₄ (a), concordance of direction of change of significantly altered metabolites (b), and concordance of direction of change of metabolites exhibiting significant differences at one of the time points (c).

Applied predictive modeling was applied to NIS (n=29) and sepsis patients (n=121). Partition trees, allowing 100 filtered predictors after t-test and FDR correction, and standard partitioning analysis, yielded a classifier comprising mean arterial pressure, temperature, epiandrosterone-sulfate, X-09789, and X-11302, reference characteristic. Accuracy was 92.7%, AUC was 95.4%, RMSE was 25.1%, PPV was 96.7% (sepsis diagnosis) and NPV was 75.9% (NIS diagnosis) (Table 9). This classifier performed poorly in cross validation performed poorly (76% accuracy, 63% AUC, and 44% RMSE). Validation in t₂₄ samples yielded 88.6% accuracy, 96.9% AUC, 28.1% RMSE, 98.1% PPV and 48.0% NPV.

Classification improved when sepsis death was omitted from the training set, leaving NIS (n=29) and sepsis survivors (n=90) (Table 9). Partition trees using K-means clustering, six filtered variables after t-test and FDR correction, and forest analysis selected body temperature, alanine, glycine, acetylcarnitine, DHEAS and X-11302 as predictive variables, reference characteristic. This classifier had 95.0% accuracy, 97.4% AUC, 26.2% RMSE and high predictive accuracy with PPV of 98.9% and NPV of 82.8%. Cross validation yielded 76% accuracy, 74% AUC and 40% RSME. In t₂₄ validation samples, accuracy was 91.3%, AUC was 97.4%, RMSE was 27.2%, PPV was 97.4% and NPV was 72.0%.

TABLE 9
PREDICTIVE MODELING FOR NIS VS. SEPSIS

| | | | | | Prediction | |
|---|---|---|---|---|---|---|
| NIS vs. Sepsis | | | | | Accuracy | |
| Comparison | Model | Accuracy | AUC | RSME | PPV | NPV |
| t0 Comparison | NIS (n = 29) vs. Sepsis (n = 121) | | | | | |
| Unsupervised | Partition | 0.9267 | 0.9543 | 0.2509 | 96.7 | 75.9 |
| | Trees | | | | | |
| Cross Validation | Partition | 0.76 ± 0.01 | 0.63 ± 0.02 | 0.44 ± 0.01 | * | * |
| | Trees | | | | | |
| | NIS (n = 25) vs. Sepsis (n = 107) | | | | | |
| t24 Validation | Partition | 0.8864 | 0.9690 | 0.2806 | 98.1 | 48.0 |
| | Trees | | | | | |
| | NIS (n = 29) vs. Sepsis Survivors (n = 90) | | | | | |
| Unsupervised | Partition | 0.9496 | 0.9762 | 0.2618 | 98.9 | 82.8 |
| | Trees | | | | | |
| Cross Validation | Partition | 0.76 ± 0.01 | 0.74 ± 0.02 | 0.40 ± 0.01 | * | * |
| | Trees | | | | | |
| | NIS (n = 25) vs. Sepsis Survivors (n = 78) | | | | | |
| t24 Validation | Partition | 0.9126 | 0.9738 | 0.2722 | 97.4 | 72.0 |
| | Trees | | | | | |

Predictive Modeling with Replication Datasets

After the replication datasets were completed predictive modeling was repeated to widen the scope of predictors for future “tuning” of the models. Two approaches were employed: Classical predictive modeling, and a Bayesian elastic net approach using beta process factor analysis and group probit regression (BPFA-GPR) (Zaas et al. 2009). In the classical modeling approach, four modeling methods were employed (JMP Genomics 4.0): 1) discriminant analysis; 2) general linear model selection; 3) logistic regression; and 4) partition trees. The results for all but logistic regression with penalized parameter reduction were poor, or exhibited poor cross validation or replication.

To distinguish among sepsis outcomes, t₀ data, including clinical parameters, was used in a training set of sepsis survivors versus sepsis death. Piperine, palmitoylcarnitine, 3-methoxytyrosine, octanoylcarnitine, clinical blood lactate, and unannotated analytes X-12775, and the single sulfated unannotated steroid X-11302 were selected, reference characteristic (Table 10). Accuracy, receiver operating characteristic area under the curve (AUC) were high (0.923, 0.957 respectively), while root mean square error (RMSE) was low (0.254). Positive predictive value (PPV) was 96.7% and negative predictive value (NPV) was 80.7%. Validity of the model was challenged in three other data sets. Accuracy and AUC remained high in these (t₂₄, Rt₀, and Rt₂₄; accuracy, 0.804, 0.784, 0.816 respectively; AUC, 0.838, 0.789, 0.784 respectively), while RMSE was low (0.379, 0.425, 0.435, respectively). PPVs were excellent (91.0° A, 94.1%, 96.7% respectively), while NPVs were reasonable (51.7%, 47.1%, 50.0%, respectively).

A model was also developed for the more clinically relevant comparison of all sepsis patients versus non-infected SIRS-positive controls (Table 10): Galactonate, uridine, maltose, glutamate, creatine and unannotated metabolite X-12644 were selected and exhibited high accuracy in the t₀, and t₂₄ datasets, reference characteristic, (0.880, 0.788, respectively; AUC, 0.927, 0.742; RMSE, 0.2841, 0.424; PPV, 95.9%, 87.9%; NPV, 55.2%, 40.0%). Results improved when confounding sepsis deaths were omitted from the analysis (sepsis diagnosis).

In another model, citrulline, laurylcamitine, androsterone sulfate, isoleucine, X-11838, X-12644, and X-11302 were selected, reference characteristic. Here, the accuracy was 0.924, 0.806, respectively; the AUC was 0.963, 0.852, respectively; RMSE was 0.247, 0.424, respectively; PPV was 94.4%, 84.6%; NPV was 84.6%, 68.0%).

In Table 10, the following definitions apply: BPFA-GPR is the B process factor analysis and group probit regression; AUC is the receiver operator characteristics area under the curve; RMSE is the root mean square error; APACHE II is the Acute Physiology And Chronic Health Evaluation Score II; and SOFA is the Sequential Organ Failure Assessment score. In Table 10, the footnotes are as follows: ¹reference characteristics: piperine, palmitoylcarnitine, 3-methoxytyrosine, octanoylcarnitine, blood lactate, unannotated LC/MS analytes X-12775 and sulfated steroid X-11302; ²reference characteristics: 8 of 11 clusters (Table 10); ³reference characteristic: creatinine, 4-vinylphenol sulfate, c-glycosyltryptophan, X-11261, X-12095, X-12100, X-13553; ⁴reference characteristics: galactonate, uridine, maltose, glutamate, creatine, X-12644; ⁵reference characteristics: 3 of 67 clusters (Table 10); ⁶reference characteristics: citrulline, laurylcamitine, androsterone sulfate, isoleucine, X-11838, X-12644, X-11302; ⁷reference characteristics: 5 of 74 cluster (Table 10).

TABLE 10
METABOLOMIC PREDICTIVE MODELS

| | | APACHE II (≧25) | | |
|---|---|---|---|---|
| | | Accuracy | PPV | NPV |
| Sepsis Prognosis | t₀ | 77.20% | 90.00% | 36.40% |
| (Day 28 death) | t₂₄ | 79.10% | 87.30% | 56.50% |
| | Rt₀ | 73.90% | 93.90% | 23.10% |
| | Rt₂₄ | 75.60% | 96.70% | 18.20% |

| | | Logistic Regression¹ | | | | |
|---|---|---|---|---|---|---|
| | | Accuracy | AUC | RMSE | PPV | NPV |
| Sepsis Prognosis | t₀ | 92.30% | 95.70% | 25.40% | 96.70% | 80.70% |
| (Day 28 death) | t₂₄ | 80.40% | 83.80% | 37.90% | 91.00% | 51.70% |
| | Rt₀ | 78.40% | 78.90% | 42.50% | 94.10% | 47.10% |
| | Rt₂₄ | 81.60% | 78.40% | 43.50% | 96.70% | 50.00% |
| | | Logistic Regression⁴ | | | | |
| | | Accuracy | AUC | RMSE | PPV | NPV |
| Sepsis Diagnosis | t₀ | 88.00% | 92.70% | 28.40% | 95.90% | 55.20% |
| (All sepsis) | t₂₄ | 80.00% | 74.20% | 42.40% | 87.90% | 40.00% |
| | | Logistic Regression⁶ | | | | |
| | | Accuracy | AUC | RMSE | PPV | NPV |
| Sepsis Diagnosis | t₀ | 92.40% | 96.30% | 24.70% | 94.40% | 86.20% |
| (Sepsis | t₂₄ | 80.60% | 85.20% | 40.00% | 84.60% | 68.00% |
| survivors) | | | | | | |

| | | | | | Capillary Lactate | | |
|---|---|---|---|---|---|---|---|
| | | SOFA (≧7) | | | (≧4.0 mg/dL) | | |
| | | Accuracy | PPV | NPV | Accuracy | PPV | NPV |
| Sepsis Prognosis | t₀ | 68.50% | 70.00% | 63.60% | 75.00% | 90.80% | 37.00% |
| (Day 28 death) | t₂₄ | 65.20% | 64.30% | 66.70% | 61.20% | 85.70% | 20.00% |
| | Rt₀ | 61.80% | 75.00% | 30.00% | 60.60% | 85.70% | 16.70% |
| | Rt₂₄ | 47.60% | 62.50% | 38.50% | 75.00% | 100.00% | 25.00% |

| | | BPFA-GPR² | | | BPFA-GPR³ | | |
|---|---|---|---|---|---|---|---|
| | | Accuracy | PPV | NPV | Accuracy | PPV | NPV |
| Sepsis Prognosis | t₀ | 98.40% | 100.00% | 93.50% | 85.10% | 94.40% | 58.10% |
| (Day 28 death) | t₂₄ | 90.70% | 97.40% | 72.40% | 87.90% | 94.90% | 69.00% |
| | Rt₀ | 76.50% | 100.00% | 29.40% | 72.50% | 94.10% | 29.40% |
| | Rt₂₄ | 77.60% | 100.00% | 31.30% | 76.90% | 100.00% | 37.50% |

| | | BPFA-GPR⁵ | | |
|---|---|---|---|---|
| | | Accuracy | PPV | NPV |
| Sepsis Diagnosis | t₀ | 88.00% | 100.00% | 38.00% |
| (All sepsis) | t₂₄ | 83.30% | 99.10% | 16.00% |

| | | BPFA-GPR⁷ | | |
|---|---|---|---|---|
| | | Accuracy | PPV | NPV |
| Sepsis Diagnosis | t₀ | 97.50% | 98.90% | 93.10% |
| (Sepsis | t₂₄ | 80.60% | 89.70% | 52.00% |
| survivors) | | | | |

For the Bayesian approach, BPFA-GPR was performed as described in the statistical analysis. In sepsis outcomes, two tests were employed using BPFA-GPR. The first limited the number of feature groups, or clusters, to a total of 11, of which eight were employed in the probit regression. The top 50 features were determined to be predictive (Table 11; t₀ and t₂₄ accuracy 98.4%, 90.7%, respectively; PPV, 100.0%, 97.4%, respectively; NPV, 93.5% and 72.4%). However, the replication study results were less impressive (Rt₀ and Rt₂₄ accuracy, 76.5%, 77.65 respectively; PPV, 100.0%, 100.0%; NPV, 29.4%, 31.3%, respectively). A second analysis was performed that increased sparseness in the model (creatinine, 4-vinylphenol sulfate, c-glycosyltryptophan, X-11261, X-12095, X-12100 and X-13553, reference characteristic). Again t₀ and t₂₄ performed well (PPV=94.4%; 94.9% respectively; NPV=58.1%; 69.0% respectively). However, While PPV remained high in the replication dataset (Rt₀=94.1%; Rt₂₄=100.0%), NPV was less impressive (Rt₀=29.4%; Rt₂₄=37.5%). For diagnosis in all sepsis patients, factor analysis revealed 67 feature groups, of which most of the top 50 were found within group 67 (Table 11). Classification results, however, were not as impressive in these groups, since the model had difficulty distinguishing non-infected SIRS-positive patients at both t₀ and t₂₄ (accuracy, 88.0%, 83.3% respectively; PPV, 100.0%, 99.1% respectively; NPV, 38.0%, 16.0%). Sepsis diagnosis in comparisons of sepsis survivors and controls was more accurate: 74 clusters were found in the factor analysis, with most of the significant features taken from the final cluster, and the top 50 were determined to be predictive, reference characteristics. (see Table 11; accuracy, PPV and NPV at t₀ of 97.5%, 98.9% and 93.1%, respectively, and 80.6%, 89.7% and 52.0% at t₂₄). The logistic regression analyses provided accurate classifiers for sepsis diagnosis and sepsis outcomes, and the Bayesian analyses provided additional predictive features that can “tune” the model for improved accuracy, sensitivity and specificity.

TABLE 11
PREDICTIVE OUTCOME

| t24 Sepsis Prognosis (Sepsis Death vs. | Regression | Feature |
|---|---|---|
| Survival) | Coefficients | Groups |
| dihydroxyacetone | 1 | 4 |
| 1-methylimidazole acetate | −0.2641 | 6 |
| palmitoylcarnitine (C16) | −0.225 | 1 |
| X-12095 | −0.1993 | 6 |
| X-02249 | 0.151 | 6 |
| 3-(4-hydroxyphenyl)lactate (HPLA) | −0.1014 | 5 |
| N-acetylneuraminate | −0.0869 | 6 |
| X-11478 | 0.0814 | 1 |
| X-11445 | 0.0794 | 6 |
| piperine | 0.0765 | 6 |
| butyrylcarnitine (C4) | −0.0763 | 6 |
| X-12217 | 0.0748 | 6 |
| 7-alpha-hydroxy-3-oxo-4-cholestenoate | −0.0707 | 1 |
| (7-HOCA) | | |
| laurylcarnitine (C12) | −0.0663 | 1 |
| 3-methoxytyrosine | −0.0643 | 1 |
| laurate (12:0) | 0.0633 | 1 |
| decanoylcarnitine (C10) | −0.0626 | 1 |
| 1-palmitoleoyl-GPC (16:1) | 0.0608 | 4 |
| X-14626 | 0.0585 | 4 |
| X-11302 | 0.0542 | 6 |
| 1-arachidonoyl-GPC (20:4) | −0.0536 | 4 |
| heme | 0.0518 | 8 |
| X-06126 | −0.0518 | 2 |
| X-11826 | 0.0515 | 6 |
| X-12695 | 0.0497 | 6 |
| gulono-1,4-lactone | 0.0486 | 6 |
| X-11809 | 0.0475 | 1 |
| 4-vinylphenolsulfate | 0.0464 | 2 |
| lactate | −0.0446 | 5 |
| X-07765 | 0.0439 | 2 |
| epiandrosterone sulfate | 0.043 | 6 |
| X-12742 | 0.0414 | 6 |
| X-11255 | 0.0413 | 4 |
| X-11546 | 0.041 | 4 |
| X-11444 | 0.0409 | 6 |
| X-12100 | 0.0403 | 2 |
| X-11450 | −0.04 | 6 |
| alanine | 0.0398 | 1 |
| pyruvate | −0.0392 | 5 |
| androsterone sulfate | 0.0371 | 6 |
| 2-methoxyacetaminophen sulfate | 0.0368 | 6 |
| biliverdin | 0.0368 | 4 |
| X-12405 | 0.036 | 2 |
| quinate | −0.0345 | 6 |
| X-11843 | −0.0344 | 6 |
| X-03056 | 0.0341 | 1 |
| X-12051 | 0.0331 | 8 |
| X-11421 | −0.0328 | 1 |
| X-13553 | −0.0328 | 2 |
| Creatinine | 0.0328 | 2 |
| | Regression | Feature |
| t24 Sepsis Diagnosis (All) | Coefficients | Groups |
| cortisol | −1 | 67 |
| creatine | −0.2639 | 15 |
| X-12802 | −0.2309 | 67 |
| laurylcarnitine (C12) | 0.1406 | 67 |
| phenol sulfate | 0.1297 | 67 |
| citrulline | 0.128 | 67 |
| acetylcarnitine (C2) | 0.1231 | 67 |
| N-acetylthreonine | −0.121 | 67 |
| X-12029 | −0.1138 | 67 |
| caprate (10:0) | −0.0909 | 67 |
| heptanoate (7:0) | −0.0881 | 67 |
| X-12644 | 0.0873 | 67 |
| allantoin | 0.0837 | 67 |
| 4-acetamidophenol | −0.0794 | 67 |
| palmitoylcarnitine (C16) | 0.0744 | 67 |
| caproate (6:0) | −0.0743 | 67 |
| 5-oxoproline | −0.0689 | 67 |
| X-14588 | −0.067 | 67 |
| 1-methylimidazoleacetate | −0.0649 | 67 |
| bilirubin (E, E) | −0.0648 | 67 |
| 1-arachidonoyl-GPE (20:4) | 0.0619 | 67 |
| hexadecanedioate (C16) | −0.0613 | 67 |
| pantothenate (Vitamin B5) | −0.0603 | 67 |
| X-12688 | −0.0586 | 67 |
| 2-hydroxypalmitate | −0.0574 | 67 |
| X-12794 | −0.0539 | 67 |
| 4-acetamidobutanoate | −0.0506 | 67 |
| arabinose | 0.0492 | 67 |
| X-11793 | −0.0484 | 67 |
| 1-palmitoyl-GPC (16:0) | 0.0482 | 67 |
| mannose | −0.0469 | 67 |
| X-12860 | 0.0466 | 67 |
| 3-(cystein-S-yl)acetaminophen | −0.0463 | 67 |
| X-07765 | −0.0456 | 67 |
| 2-hydroxybutyrate (AHB) | 0.0447 | 67 |
| X-12092 | −0.0445 | 67 |
| malate | 0.0441 | 67 |
| phosphate | 0.0431 | 67 |
| tyrosine | 0.0419 | 67 |
| C-glycosyltryptophan | 0.0417 | 67 |
| X-12442 | 0.0416 | 67 |
| lysine | 0.0373 | 65 |
| X-11838 | −0.0369 | 67 |
| docosapentaenoate (DPA; 22:5n3) | 0.0366 | 67 |
| X-11423 | 0.036 | 67 |
| erythronate | −0.035 | 67 |
| 5-dodecenoate (12:1n7) | −0.0344 | 67 |
| X-11381 | −0.033 | 67 |
| erythrose | 0.0322 | 67 |
| 3-methyl-2-oxovalerate | 0.0319 | 67 |
| | Normalized | |
| t24 | Regression | Feature |
| Sepsis Diagnosis (Sepsis Survivors) | Coefficients | Groups |
| X-11302 | −1 | 15 |
| creatine | −0.9463 | 5 |
| decanoylcarnitine (C10) | 0.2886 | 74 |
| laurylcarnitine (C12) | 0.2342 | 74 |
| allantoin | 0.2096 | 74 |
| X-12855 | 0.2084 | 74 |
| acetylcarnitine (C2) | 0.2053 | 74 |
| citrulline | 0.2034 | 74 |
| X-11793 | −0.1812 | 74 |
| hexadecanedioate (C16) | −0.1657 | 74 |
| X-12465 | 0.1649 | 74 |
| caprate (10:0) | −0.1637 | 74 |
| X-12644 | 0.1511 | 74 |
| X-12794 | −0.149 | 74 |
| 5-dodecenoate (12:1n7) | −0.1396 | 5 |
| 3-methyl-2-oxovalerate | 0.1378 | 74 |
| propionylcarnitine (C3) | 0.1332 | 74 |
| cortisol | −0.1313 | 72 |
| docosahexaenoate (DHA; 22:6n3) | 0.1312 | 74 |
| threonine | 0.1155 | 74 |
| stearidonate (18:4n3) | 0.1114 | 74 |
| N-acetylornithine | −0.1094 | 74 |
| 4-acetaminophen sulfate | −0.1083 | 74 |
| glycerol | 0.1082 | 74 |
| phenylalanine | −0.1059 | 74 |
| caproate (6:0) | −0.1021 | 74 |
| phosphate | 0.0985 | 74 |
| heptanoate (7:0) | −0.0975 | 74 |
| docosapentaenoate (DPA; 22:5n3) | 0.0963 | 74 |
| caprylate (8:0) | −0.0888 | 74 |
| 4-acetamidobutanoate | −0.0887 | 74 |
| X-12206 | 0.0864 | 74 |
| erythronate | −0.0854 | 74 |
| laurate (12:0) | −0.0841 | 74 |
| 1-palmitoyl-GPC (16:0) | 0.0821 | 74 |
| glycerate | 0.08 | 74 |
| X-11421 | 0.0754 | 74 |
| X-12802 | −0.0739 | 74 |
| octanoylcarnitine (C8) | 0.0725 | 74 |
| 2-aminobutyrate | 0.0709 | 74 |
| bilirubin (E, E) | −0.068 | 5 |
| myo-inositol | 0.0664 | 74 |
| X-07765 | −0.066 | 56 |
| creatinine | −0.0633 | 74 |
| leucine | −0.063 | 72 |
| citrate | 0.0616 | 74 |
| X-11423 | 0.0614 | 74 |
| isoleucine | 0.0605 | 74 |
| proline | 0.0604 | 74 |
| X-12405 | −0.0595 | 74 |

### Changes in Plasma Proteins Relevant to Metabolic Perturbation

Peptides corresponding to 2,583 proteins were measured with linear ion-trap MS coupled with HPLC in 150 t₀ plasma samples. No statistically significant differences were observed between NIS and UCS, or between UCS, SS and SShock. However, 616 proteins differed significantly between UCS and SD (weighted ANOVA, 5% FDR). Of these, 527 with annotations were mapped to metabolic pathways; 25 of these were salient to concurrent metabolite changes and are reported here. Consistent with altered mitochondrial integrity were elevated t₀ plasma levels of (a) mitochondrial citrate synthase (1.30-fold in SD versus UCS), (b) succinate dehydrogenase (2.44-fold), (c) glyceraldehyde-3-phosphate dehydrogenase (1.41-fold), (d) the alpha subunit of ATP synthase (1.23-fold), and (e) pantothenate kinase 4 (1.62-fold), reference characteristic. Consistent with altered glycogenolysis were decreased levels of (a) glycogenin (0.53-fold in SD versus UCS), (b) glycogen synthase kinase-3 alpha (0.77-fold), and (c) neutral alpha glucosidase C (0.74-fold) reference characteristic. Consistent with perturbed lipid transport and catabolism were decreases in the levels of six plasma apolipoproteins SD (1) A-I (0.68-fold in SD versus UCS), (2) A-II (0.52-fold), (3) A-IV (0.78-fold), (4) C-IV (0.80-fold), (5) L-1 (0.78-fold), and (6) steroid carrier protein 2 (0.73-fold)) reference characteristic. Also consistent with perturbed lipid transport and catabolism were alterations in three lipid transfer enzymes: (1) lysocardiolipin acyltransferase 1 (0.71-fold in SD versus UCS), (2) phospholipid-transporting ATPase ATP11B (1.57-fold), and (3) fatty acid binding protein 4 (1.54-fold)). Consistent with altered steroid synthesis was a notable increase in 3-hydroxy-3-methylglutaryl-Coenzyme A reductase (HMG-CoA reductase) (3.4-fold increase in SD versus UCS). Indicative of energy stress were increases in the levels of adenylate cyclase type 3 (1.88-fold increase in SD versus UCS), AMPD3 (0.46-fold), cAMP-specific 3′,5′-cyclic phosphodiesterase 4D (0.53-fold), and 5′-nucleotidase, cytosolic III-like (0.59-fold) reference characteristic.

### (IV). Discussion of Examples

Host metabolic responses to sepsis and its complications were defined using unbiased plasma metabolic profiles of 150 patients at time of presentation and 24 hours later. No significant differences were detected between sepsis survivors with uncomplicated courses and those who progressed to SS or SShock within three days. Thus, adult sepsis survivors appear to be metabolically homogeneous, irrespective of clinical course or etiologic agent. In contrast, numerous metabolic differences were detected at presentation and 24 hours later between sepsis survivors, 28-day sepsis deaths and NIS. Metabolic differences between adults with NIS and sepsis survivors are inferred to represent a beneficial or physiologic host response to sepsis. Metabolic differences between sepsis survivors and deaths at time of presentation and at 24-hours were essentially identical and indicate a metabolic basis for sepsis death to which a subset of patients appears programmed. The potential diagnostic, prognostic and therapeutic implications of these differences for management of sepsis are profound.

Host metabolic response to sepsis was extensive. At time of presentation, plasma of sepsis survivors and NIS patients exhibited 40 annotated, metabolic differences, reference characteristic. Most of these reflected increased plasma androgenic steroids and decreased plasma intermediates in glycolysis, the TCA cycle, and fatty acid and amino acid catabolism, reference characteristic. Many of the metabolic differences between sepsis survivors and NIS were supported by previous studies: Hypoglycemia has previously been reported in sepsis and peripheral and hepatic insulin sensitivity has been observed early in uncomplicated sepsis (van der Crabben et al., 2009; Alamgir et al., 2006). Protein catabolism, as measured by weight loss, urinary nitrogen loss and conversion of alanine to glucose, is markedly elevated in UCS (Long et al., 1977). However, the literature lacks consensus regarding plasma amino acid concentrations in sepsis, which reflect variation in sampling times during sepsis progression (Clowes et al., 1980; Vente et al., 1989; Druml et al., 2001; Wannemacher 1977; Freund et al., 1979; Freund et al., 1978; Ploder et al., 2008). Moreover, because plasma amino acids constitute a small pool with rapid turn-over, changes in plasma levels need to be interpreted with caution (Wilmore et al., 1980). The data indicate that aerobic glycolysis, gluconeogenesis, β-oxidation of fatty acids and the TCA cycle are increased in sepsis survivors, reference characteristic. These metabolic differences are likely to represent physiologic responses to host energy stress that are reflected by SIRS criteria and can be necessary for survival. This is in agreement with marked elevation of metabolic rate in uncomplicated sepsis (Alamgir et al., 2006; Kreymann et al., 1993) and with significant pyrexia in the UCS group at presentation.

Three lysophosphatidylcholines (LPCs) were significantly lower in UCS/sepsis survivors than NIS at t₀, reference characteristic. LPCs are bioactive lipids generated in plasma by phospholipase A₂ (PLA₂). LPCs function in immune regulation (Kabarowski et al., 2002), monocyte chemotaxis (Jing et al., 2000), macrophage activation (Yamamoto et al., 1991) and are ligands for the G protein-coupled receptor, G2A, resulting in immunosuppression (Kabarowksi et al., 2001). LPC levels decrease in early sepsis (Drobnik et al., 2003; Lissauer et al., 2007) and the degree of change is predictive of mortality (Drobnik et al., 2003). Decreased plasma LPCs reflect either reduced PLA₂ activity or increased activity of enzymes that convert LPC to lysophosphatidic acid (Umezu-Goto et al., 2002; Tokumura et al., 2002; Clair et al., 2003). PLA₂ has potent bactericidal activity (Beers et al., 2002; Koduri et al., 2002; Weinrauch et al., 1996). LPC therapy has been proposed in sepsis (Murch et al., 2006; Yan et al., 2004; Chen et al., 2005; Blondeau et al., 2002) as a non-competitive inhibitor of PLA₂ (Cunningham et al., 2008).

Biomarkers that supplement clinical criteria are needed for rapid diagnosis of sepsis. A positive diagnostic biomarker result is anticipated to be useful for early selection of patients for antibiotic therapy and early goal directed therapy. A negative diagnostic biomarker result avoids empiric antibiotic therapy and prompts caregivers to pursue differential diagnosis. The specificity of clinical criteria alone for diagnosis of sepsis (evidence of infection and presence of at least 2 SIRS criteria) in the CAPSOD study was 85% at time of presentation (Glickman et al., 2010). Predictive models were developed that incorporated metabolic and clinical differences between sepsis survivors and NIS patients. A six-factor partition trees classifier had an AUC of 97.4% in training and replication sets. This is superior to previous biomarkers for sepsis diagnosis, such as procalcitonin (Brunkhorst et al., 2000; Cheval et al., 2000; Rau et al., 2000; Reith et al., 2000) or cytokine combinations (Oberhoffer et al., 2000; Shapiro et al., 2009; Kingsmore et al., 2005). This diagnostic classifier was also conceptually more satisfactory since it was the result of an unbiased, comprehensive analysis and since the individual factors (body temperature, alanine, glycine, acetylcarnitine, DHEAS and X-11302) fit a unifying clinico-metabolomic hypothesis, reference characteristic.

Numerous metabolites differentiated sepsis patients who survived from those who died by day 28. These differences were evident at presentation and became more pronounced 24 hours. Given the relatively long time between detection of metabolic divergence and sepsis death (median day 8), it appears that the initial metabolic response to sepsis programs the host for death or survival. The metabolic pathway alterations that distinguished sepsis survivors and deaths were essentially the reverse of those that distinguished sepsis survivors from NIS patients: Thus, while sepsis survivors and deaths both exhibited metabolic changes reflective of lipolysis and protein catabolism, sepsis death was uniquely distinguished by accumulation in plasma of downstream intermediates that are typically within mitochondria (amino acid catabolites, carnitine esters, fatty acids, malate and phosphate), increased anaerobic glycolysis (evidenced by lactate) and tRNA turnover, reference characteristic. There is considerable literature support for mitochondrial loss and dysfunction in sepsis patients with high mortality (Vanhorebeek et al., 2005; Brealey et al., 2002; Fredriksson et al., 2006; Fredriksson et al. 2008). A clinical correlate of mitochondrial dysfunction at presentation in patients that later die was absence of fever. The primary origin of mitochondrial differences between sepsis survivors and deaths is unclear and merits further functional and genetic analysis.

Biomarkers that supplement clinical indices are needed for prognostic assessment and personalized treatment of sepsis. Prognostic biomarkers allow early nomination of patients for hospital admission, intensive care, early goal directed therapy or treatment with activated protein C, intensive glycemic control or corticosteroid administration. Prognostic biomarkers also provide metabolic surrogate end-points by which to assess efficacy of interventions and guide treatment duration and intensity. The metabolic pathway alterations that distinguish sepsis survivors and deaths provide a target profile for therapeutic interventions in patients at high risk of sepsis death. The current gold standards for prognostic assessment in sepsis are the Sequential Organ Failure Assessment (SOFA) and the Acute Physiology and Chronic Health Evaluation (APACHE II) tests (Knaus et al., 1985; Vincent et al., 1996). However, neither is used routinely since they are time consuming and contain subjective components with variable interpretation. The two clinico-metabolomic biomarker panels described herein, reference characteristic, (mean arterial pressure, serum sodium, hematocrit, HPLA, piperine, and γ-tocopherol); and (temperature, alanine, glycine, acetylcarnitine, DHEAS, and X-11302) had considerably better performance in distinguishing sepsis survivors from sepsis deaths than SOFA or APACHE II (AUC>95% in training and validation sets, compared with <75%). Since 28-day mortality in the CAPSOD cohort (n=1152) was 7%, the availability of an effective prognostic indicator in sepsis can greatly improve the cost-effectiveness of expensive interventions such as EGDT or treatment with APC. Furthermore, such prognostic indicators add value in dynamic assessment of efficacy, allowing tailoring of therapy to patient needs. Such adjustments can improve the effectiveness of such treatments and/or reduce the risk of adverse events, such as cerebrovascular accidents with APC therapy. In addition, it has been difficult to assess the benefit of novel interventions in sepsis, such as intensive glycemic control or administration of exogenous steroids, in the absence of a biochemical surrogate marker. The prognostic assessment biomarker panels described herein provide both a patient selection mechanism for novel sepsis therapies and a surrogate marker for efficacy. Several of the individual components of the prognostic classifiers have previously been described. For example, clinical correlates of mitochondrial dysfunction at presentation in patients that die were absence of fever and low mean arterial pressure. These have previously been identified in the CAPSOD cohort as predictive of adverse outcomes in sepsis (Glickman et al., 2010).

When required for energy, stored triacylglycerols are cleaved by hormone-regulated lipases, releasing fatty acids and glycerol into plasma. Upon cellular uptake, fatty acids are activated with CoA and cross the outer mitochondrial membrane, where the acyl group is transferred to carnitine to form acylcarnitine (Lieberman et al., 2009). Acylcarnitines are shuttled across the inner mitochondrial membrane by a translocase and the acyl group is transferred back to CoA (Berg et al., 2002). In the mitochondrion, fatty acids are metabolized by 13-oxidation into acetyl-CoA, which enters the TCA cycle. Acylcarnitines are logical candidates for implication in sepsis outcome, given their requirement for mitochondrial uptake of fatty acids for 13-oxidation. Variable changes in acylcarnitine levels have been reported in humans and in animal models of sepsis (Linz et al., 1995; Davis et al., 1991; Lanza-Jacoby et al., 1988; Lanza-Jacoby et al., 1991; Keskin et al., 1997; Nanni et al., 1985; Dugas et al., 2000; Giovannini et al., 1983). Increased plasma levels of acylcarnitines in SD are likely to result from impaired mitochondrial oxidation of fatty acids, resulting in leakage of esterified carnitines into plasma. Levels of free fatty acids and ketone bodies were relatively unchanged in SD, lending some support to deficient β-oxidation in a setting of high energy need. Impaired fatty acid β-oxidation (Giovannini et al., 1983) and mitochondrial function (Brealey et al., 2002; Fukumoto et al., 2003) have been reported during sepsis. In plasma, acylcarnitines have deleterious effects, including reduced levels of proinflammatory cytokines (Winter et al. 1995) and cardiotoxicity.

Consistent with these results were a dramatic changes in proteins that are involved in lipid transport and acyltransfereases, reference characteristic. These include group-specific component (gc-globin; −1.21) also known as vitamin D binding protein, serum amyloid A 1 (SAA1; −1.14), apolipoprotein A-I (APOA1; −1.48), apolipoprotein A-II (APOA2; −1.91), apoliprotein LI (APOL1; −1.28), apoliporotein C-1V (APOC4; −1.25), transthyretin (TTR; −1.64), reference characteristic. APO1 levels have been noted to decrease during acute inflammatory responses (Chait et al., 2005). Further, APO1 can bind to LPS and has been shown to be protective from LPS induced SShock in a mouse model. It is also interesting to note that activated protein C (APC) treatment increases APO1 (Sharma et al., 2008). Gc-globin is a member of the albumin family that binds to vitamin D and its plasma metabolites and transports them to target tissues. Gc-globin also binds to extracellular actin released by necrotic cells protecting against DIC induced by polymerized actin and can also scavenge endotoxin (Meier et al., 2006). Several clinical studies have shown that low gc-globin is associated with poor prognosis (reviewed in Meier et al., 2006). Decreased levels of TTR has previously been reported in sepsis (Biolo et al., 1997), and in odontogenic infections the length of hospital stay has been negatively correlated to the concentration of TTR (Cunningham et al., 2006).

Consistent with the decrease in lipoproteins is the corollary decrease in a number of proteins involved in fatty acid metabolism, including: paraoxonase 1 (PON1; −1.25), lysocardiolipin acyltransferase (LCLAT1; −1.41), glycosylphosphatidylinositol specific phospholipase DI (GPLD1; −1.18), ectonucleotide pyrophosphatase/phosphodiesterase 2 (ENPP2; −1.59), reference characteristic. PON1 is an arylesterase that resides primarily on HDL and hydrolyzes paroxon to produce p-nitrophenol. Decrease in acyltransferase has been noted in septic rats and treatments with dexamethasone increased the expression of bile acyltransferease and improved outcome (Chen et al., 2008). PON1 is believed to have a protective role in vascular disease (James 2006). GPLD1 hydrolyzes the inositol phosphate linkage in proteins anchored by phosphtidylinositol glycans, releasing the protein from the plasma membrane serum increases are associated with insulin resistance (Kurtz et al., 2004). ENPP2, also known as lysoPLD functions as a phosphodiesterase and a phospholipase catalyzing the production of lysophosphatidylcholine (LPC) into lysophosphatidic acid (LPA) in extracellular fluids (Li and Zhang, 2009; Nakasaki et al., 2008). LPC's are a bioactive lipid generated by PLA2-mediated hydrolysis of phosphatidylcholine and can inhibit apoptosis and neuritogenesis (Yun et al., 2006).

Appreciable amounts of plasma LPC are formed by lecithin:cholesterol acyltransferase (LCAT), which is secreted from the liver. LCAT catalyzes the transfer of fatty acids from the sn-2 position of phosphatidylcholine to cholesterol, resulting in the formation of LPC and cholesterol ester (Glomset 1968). Cholesterol esterification in plasma is essential for maintaining the gradient of free cholesterol in blood. Thus, LCAT plays an important role in reverse cholesterol transport by regulating the transport of cholesterol from peripheral tissues to HDL (Glomset 1968). Profound alterations in lipid metabolism are known to occur during infection and inflammation, including a reduction in plasma concentrations of total cholesterol, HDL, LDL and phospholipids (Pruzanski et al., 2000; Khovidhunkit et al., 2000; Levels et al., 2007). As a result of decreased LCAT activity and the loss of the free cholesterol gradient in plasma, the suppression of reverse cholesterol transport can account for altered lipid homeostasis in sepsis. Indeed LCAT activity was found to be depressed in human plasma during sepsis (Levels et al., 2007; Ly et al., 1995). The fact that plasma levels of LPCs were significantly lower in sepsis survivors compared to NIS is consistent with the biochemical changes that resulted from sepsis-induced decreases in LCAT levels and/or activity.

Sepsis survivors had increased anabolic steroids DHEAS, epiandrosterone-sulfate, androsterone-sulfate, and two unnamed sulfated steroid derivatives (X-11245 and X-11302) compared to NIS, reference characteristic. Further, epiandrosterone-sulfate (0.76-fold; p=0.018), androsterone-sulfate (0.74-fold; p=0.028), X-11302 (0.73-fold; p=0.009) barely missed significance after FDR correction in the comparison of SD and sepsis survivors, reference characteristic. HMG-CoA reductase is an integral Membrane protein of the smooth ER which controls the rate-limiting step in cholesterol synthesis (Nelson et al., 2008). Statins, which have anti-inflammatory properties and inhibit HMG-CoA reductase, have been tested as a therapy in sepsis (Gao et al., 2008; Mackenzie and Lever, 2007). Increases are also seen in aryl-sulfatase H (ARSH; 1.25-fold change). 24-dehydrocholesterol reductase (DHCR24) is reduced (1.37-fold). While little is known about ARSH, arylsulfatases catalyze the hydrolysis of sulfate ester bonds and are involved in hormone biosynthesis, modulating cell signaling and degradation of macromolecules (Sardiello et al., 2005). DHCR24 is an FAD-dependant oxidoreductase which catalyzes the reduction of delta-24 double bonds of sterol intermediates during steroid biosynthesis. (Peri et al., 2005). Plasma levels of DHEAS have been reported to decrease in sepsis (Parker et al., 1985; Beishuizen et al., 2002; Marx et al., 2003). DHEAS is the most abundant adrenal steroid in circulation (Ebeling and Koivisto, 1994) and is present at much greater levels than the non-sulfated form, DHEA. While the adrenals secrete both DHEA and DHEAS, conversion between the two forms is catalyzed by DHEA sulfotransferase (SULT2A1) (Hammer et al., 2005), an enzyme that has been shown to be down-regulated in a rodent sepsis model (Kim et al., 2002). DHEA is considered the biologically active form, functioning as a potent stimulator of the immune system and eliciting effects indirectly via conversion to androgens and estrogens. In animal models of sepsis, exogenous administration of DHEA produced beneficial effects and reduced mortality (Ebeling and Koivisto, 1994; Oberbeck et al., 2001; Angele et al., 1998; Catania et al., 1999; Ben-Nathan et al., 1999; van Griensven et al., 2002; Hildebrand et al., 2003; Hildebrand et al., 2004). Coupled with the reported increase in cortisol, the decrease in circulating DHEAS levels in sepsis has been interpreted as a stress-induced shift from adrenal androgen to glucocorticoid synthesis (Parker et al., 1985; Beishuizen et al., 2002; Vermes and Beishuizen, 2001). In contrast to published data however, DHEAS levels in the present study were observed to increase in UCS. It is possible that differences in the sampling times in relation to sepsis progression account for the discrepancy between this and previously published studies. Other factors, such as the use of dopamine, can contribute to the lower levels measured in published literature (Beishuizen et al., 2002; Van den Berghe et al., 1995). The increase in DHEAS observed in UCS can reflect a homeostatic attempt to boost immune cell function and counteract the immunosuppressant effects of cortisol. Other sulfated steroids exhibited similar changes in levels. Namely, these sulfated steroids were present at greater levels in UCS compared to NIS. Epiandrosterone sulfate is the sulfated form of epiandrosterone, a metabolite of DHEA. Epiandrosterone is a weak androgen, but also functions to inhibit the pentose phosphate pathway and reduce intracellular levels of NADPH (Gordon et al., 1995). NADPH provides reducing equivalents for lipid synthesis, cholesterol synthesis, and fatty acid chain elongation. Androsterone (sulfate) is a breakdown metabolite of testosterone but can also be metabolized from DHEA, dihydrotestosterone (DHT), and androstenedione. Androsterone is a ligand of the farnesoid X receptor (FXR), which plays a critical role in bile acid metabolism (Wang et al., 2006). X-11245 is thought to be a di-sulfated steroid and X-11302 a singly sulfated steroid based on their chromatographic elution times, MS/MS fragmentation, and masses. The functional significance of androsterone sulfate, epiandrosterone sulfate, and the two unnamed sulfated steroid in relation to sepsis is unclear.

HPLA and HPA are produced through anaerobic degradation of aromatic amino acids or dietary polyphenolic compounds by intestinal bacteria (Feotcheva et al., 2008). Increased HPLA has previously been documented in sera of sepsis patients, with the highest levels in non-survivors (Kodakova and Beloborodova, 2007). Further, HPLA and HPA are indicated to contribute to mitochondrial dysfunction in sepsis (Feotcheva et al., 2008; Kodakova and Beloborodova, 200). 3-hydroxy-2-ethylpropionate is an intermediate of isoleucine oxidation (Korman et al., 2005). Dimethylarginine is formed post-translationally in arginine-containing proteins, and its presence in plasma indicates protein catabolism (Teerlink 2004). Dimethylargenine has previously been reported to be elevated in serum of patients with SShock and to be greater in SD than sepsis survivors (O'Dwyer et al., 2006). Dimethylargenine inhibits nitric oxide synthases that regulate vascular tone (Kilbourn 1998; Boyle et al., 2000) and has been proposed to impair nitric oxide-dependent host defenses (O'Dwyer et al., 2006; Boger 2006). Prolylhydroxyproline is a dipeptide marker of muscle and bone collagen degradation (Husek et al., 2008). Markers of collagen metabolism have previously been observed to increase in patients with severe sepsis and were higher in non-survivors of sepsis when compared with survivors (Gaddnas et al., 2008). The presence in blood of acetylated amino acids, such as N-acetylglycine, N-acetylthreonine and N-acetylaspartate, reflects hydrolysis of post-translationally acetylated proteins. These findings are consistent with increased muscle protein catabolism (Cooney et al., 1997) and collagen degradation that have been reported with severe sepsis.

It has been hypothesized that transfer of FAs to carnitine may be to free the CoASH pool (reviewed in Hoppel (Hoppel, 2003)). This buffering capacity has been demonstrated during exercise when acetyl-carnitine accumulates during high-intensity exercise. Carnitines have also been suggested as a method for removal of acyl groups. The accumulation of propionate or propionyl-CoA can disrupt cellular metabolism by inhibiting short-chain FA oxidation due to the loss of free CoASH. An increase of C10orf129, also known as acyl-CoA synthetase, mitochondrial 6 (ACSM6), was found in sepsis survivors and sepsis death as compared to NIS, reference characteristic. Gene Ontology states the protein has butyl-CoA ligase activity, in other words suggesting it can bind Coenzyme A to branched chain-amino acids to allow for β-oxidation. There was a strong positive correlation of ACSM6 to branched chain acyl-carnitine esters tigylcamitine, butyrylcamitine, isobutyrylcarnitine, 2-methylbutyroylcamitine and hydroxyisovaleroylcamitine and branched-chain amino acid β-hydroyisovalerate, reference characteristic. The results suggest a predictive marker for sepsis, sepsis progression as well as a mechanistic target in sepsis induced mitochondrial dysfunction. (See also Hoppel, C., 2003, The role of carnitine in normal and altered fatty acid metabolism: Am J Kidney Dis, v. 41, p. S4-12.0)

TABLE 12
CORRELATION OF C10orf129 TO BRANCHED COMPOUNDS

| | | t0 Pearson | t0-Log10 |
|---|---|---|---|
| Metabolite | Gene Symbol | Correlation | p-value |
| Tigylcarnitine | ACSM6 | 0.322 | 4.236 |
| 2-methylbutyroylcarnitine | ACSM6 | 0.319 | 4.169 |
| Butyrylcarnitine | ACSM6 | 0.312 | 3.994 |
| hydroxyisovaleroylcarnitine | ACSM6 | 0.282 | 3.336 |
| Isobutyrylcarnitine | ACSM6 | 0.279 | 3.262 |
| β-hydroxyisovalerate | ACSM6 | 0.227 | 2.290 |

While several embodiments of the present invention have been shown and described, it is to be understood that many changes and modifications can be made.

1. A method of diagnosing sepsis in a human subject comprising,
a) measuring the amount or level of one or more clinico-metabolomic classifiers of a human subject to determine a value for the clinico-metabolomic classifier;
b) comparing the value of one or more clinico-metabolomic classifiers of the subject to
(i) a reference characteristic that was determined from the same clinico-metabolomic classifier obtained from subjects with sepsis, and to
(ii) a reference characteristic that was determined from the same clinico-metabolomic classifier obtained from non-infected subjects with systemic inflammatory response criteria; and
c) diagnosing the subject as having sepsis if the value of one or more clinico-metabolomic classifiers measured is found to be similar to the reference characteristic of subjects with sepsis.

2. The method of claim 1, further comprising optionally diagnosing the subject as having non-infected systemic inflammatory response if the value of one or more clinico-metabolomic classifiers measured is found to be similar to the reference characteristic of subjects with non-infected systemic inflammatory response.

3. The method of claim 1, wherein at least one clinico-metabolomic classifier measured is 10-undecenoate, 1-arachidonoyl-GPE (20:4), 1-methylimidazoleacetate, 1-palmitoleoylglycerophosphocholine (1-palmitoleoyl-GPC, C16:1), 1-palmitoyl-GPC (16:0), 2-hydroxybutyrate (AHB), 2-hydroxypalmitate, 3-(cystein-5-yl)acetaminophen, 3-methyl-2-oxovalerate, 4-acetamidobutanoate, 4-acetamidophenol, 5-dodecenoate (12:1n7), 5-oxoproline, acetylcarnitine (C2), allantoin, androsterone-sulfate, arabinose, bilirubin (E,E), caprate (10:0), caproate (6:0), C-glycosyltryptophan, citrulline, cortisol, creatine, dihydroepiandrosterone-sulfate (DHEAS), docosapentaenoate (DPA; 22:5n3), epiandrosterone-sulfate, erythronate, erythrose, fructose, galactonate, glutamate, glycerol-3-phosphate, heptanoate (7:0), hexadecanedioate (C16), indolepropionate, laurylcarnitine, lysine, malate, maltose, mannose, N-acetylthreonine, palmitoylcarnitine (C16), pantothenate (Vitamin B5), phenol sulfate, phosphate, serine, tyrosine, uridine, X-07765, X-09789, X-11302, X-11381, X-11423, X-11793, X-11838, X-12029, X-12092, X-12442, X-12644, X-12644, X-12688, X-12794, X-12802, X-12860, X-14588, temperature, mean arterial pressure, or bilirubin.

4. The method of claim 2, wherein sepsis is diagnosed using at least five clinico-metabolomic classifiers.

5. A method of diagnosing sepsis in a human subject presenting with suspected sepsis, comprises
a) testing a human subject for one or more clinico-metabolomic classifiers to determine the amount or level of one or more clinico-metabolomic classifiers in the subject;
b) comparing the amount or level of one or more clinico-metabolomic classifiers from the subject to
(i) a reference characteristic that was determined from the same clinico-metabolomic classifier obtained from subjects with sepsis who survive, and to
(ii) a reference characteristic that was determined from the same clinico-metabolomic classifier obtained from non-infected subjects with systemic inflammatory response criteria; and
c) diagnosing the subject as having sepsis if the amount or level of one or more clinico-metabolomic classifiers tested is found within the reference characteristic of subjects with sepsis who survive.

6. The method of claim 5, wherein at least one clinico-metabolomic classifier is 1-palmitoleoyl-GPC, 1-palmitoyl-GPC (16:0), 2-aminobutyrate, 3-methyl-2-oxovalerate, 4-acetamidobutanoate, 4-acetaminophen sulfate, 5-dodecenoate (12:1n7), acetylcarnitine, alanine, allantoin, androsterone sulfate, bilirubin (E,E), caprate, caproate, caprylate, citrate, citrulline, cortisol, creatine, creatinine, decanoylcarnitine, DHEAS, docosahexaenoate (DHA; 22:6n3), docosapentaenoate (DPA; 22:5n3), erythronate, fructose, glycerate, glycerol, glycine, heptanoate, hexadecanedioate, isoleucine, lactate, laurate, laurylcarnitine, leucine, maltose, myo-inositol, N-acetylornithine, octanoylcarnitine, phenylalanine, phosphate, proline, propionylcarnitine, pyruvate, serine, stearidonate (18:4n3), threonine, X-07765, X-11302, X-11421, X-11423, X-11793, X-11838, X-12206, X-12405, X-12465, X-12644, X-12794, X-12802, X-12855, age, body temperature, mean arterial pressure, age, or platelet count.

7. The method of claim 5, wherein sepsis is diagnosed using at least five clinico-metabolomic classifiers.

8. The method of claim 5, wherein sepsis is diagnosed using at least six clinico-metabolomic classifiers comprising body temperature, alanine, glycine, acetylcarnitine, DHEAS, and X-11302.

9. A method of diagnosing sepsis by identifying a subject at high risk of sepsis death comprising,
a) testing a human subject for one or more clinico-metabolomic classifiers to determine a value of one or more clinico-metabolomic classifiers in the subject;
b) comparing the value of one or more clinico-metabolomic classifier from the subject to
(i) a reference characteristic that was determined from the same clinico-metabolomic classifier obtained from subjects with sepsis that died within 28 days of presentation, and to
(ii) a reference characteristic that was determined from the same clinico-metabolomic classifier obtained from subjects with sepsis that did not die; and
c) diagnosing the subject as having a high risk of sepsis death if the value of one or more clinico-metabolomic classifiers tested is found within the reference characteristic of subjects with sepsis who die.

10. The method of claim 9, wherein at least one clinico-metabolomic classifier is 1-arachidonoyl-GPC, 1-methylimidazole acetate, 1-palmitoleoyl-GPC, 2-methoxyacetaminophen sulfate, 3-(4-hydroxyphenyl)lactate (HPLA), 3-methoxytyrosine, 3-methylhistidine, 4-vinylphenol sulfate, 7-alpha-hydroxy-3-oxo-4-cholestenoate (7-HOCA), acetylcarnitine, alanine, androsterone sulfate, biliverdin, butyrylcarnitine, caproate, c-glycosyltryptophan, chenodeoxycholate, creatinine, decanoylcarnitine, dihydroxyacetone, epiandrosterone sulfate, gulono-1,4-lactone, heme, lactate, laurate, laurylcarnitine, N-acetylneuraminate, octanoylcarnitine, palmitoylcarnitine, piperine, piperine, propionylcarnitine, pyruvate, quinate, γ-tocopherol, X-02249, X-03056, X-06126, X-07765, X-10395, X-11255, X-11261, X-11302, X-11421, X-11444, X-11445, X-11450, X-11478, X-11538, X-11546, X-11809, X-11826, X-11843, X-11908, X-12051, X-12095, X-12100, X-12217, X-12405, X-12695, X-12742, X-12755, X-13553, X-14626 sodium, hematocrit, mean arterial pressure, or age.

11. The method of claim 9, wherein at least seven clinico-metabolomic classifiers are tested and comprise sodium, lactate, age, hematocrit, HPLA, 3-methoxytyrosine, and X-11302.

12. The method of claim 9, wherein at least one clinico-metabolomic classifier value is tested from a sample of the subject's blood, serum, or plasma.

13. A method of diagnosing sepsis by identifying a subject at high risk of sepsis death comprising
a) testing a subject for one or more clinico-metabolomic classifiers to determine the amount or level of one or more clinico-metabolomic classifiers in the subject to determine a value for the clinico-metabolomic classifier;
b) comparing the value of one or more clinico-metabolomic classifier from the subject to
(i) a reference characteristic that was determined from the same clinico-metabolomic classifier obtained from subjects with sepsis that died within 28 days of presentation, and to
(ii) a reference characteristic that was determined from the same clinico-metabolomic classifier obtained from non-infected subjects with systemic inflammatory response criteria; and
c) diagnosing the subject as having a high risk of sepsis death if the value of one or more clinico-metabolomic classifiers tested is found to be similar to the reference characteristic of subjects with high risk of sepsis death.

14. The method of claim 13, further comprising optionally diagnosing the subject as having non-infected systemic inflammatory response if the value of one or more clinico-metabolomic classifiers measured is found to be similar to the reference characteristic of subjects with non-infected systemic inflammatory response.

15. The method of claim 13, wherein at least one clinico-metabolomic classifier is 3-(4-hydroxyphenyl)lactate (HPLA), 3-methoxytyrosine, piperine, 3-methylhistidine, lactate, caproate, acetylcarnitine, octanoylcarntine, decanoylcarnitine, propionylcarnitine, creatinine, γ-tocopherol, chenodeoxycholate, X-10395, X-11261, X-11538, X-11908, X-12095, X-12100, X-12775, X-11302, X-13553, heme, sodium, hematocrit, mean arterial pressure, body temperature, age, or respiratory rate.

16. The method of claim 13, wherein at least six clinico-metabolomic classifiers are tested and comprise mean arterial pressure, sodium, hematocrit, HPLA, piperine, and γ-tocopherol.

17. A kit for diagnosing sepsis in a human subject comprising: a reagent for detecting a clinico-metabolomic classifier, wherein the clinico-metabolomic classifier is 1-palmitoleoyl-GPC, 1-palmitoyl-GPC (16:0), 2-aminobutyrate, 3-methyl-2-oxovalerate, 4-acetamidobutanoate, 4-acetaminophen sulfate, 5-dodecenoate (12:1n7), acetylcarnitine, alanine, allantoin, androsterone sulfate, bilirubin (E,E), caprate, caproate, caprylate, citrate, citrulline, cortisol, creatine, creatinine, decanoylcarnitine, DHEAS, docosahexaenoate (DHA; 22:6n3), docosapentaenoate (DPA; 22:5n3), erythronate, fructose, glycerate, glycerol, glycine, heptanoate, hexadecanedioate, isoleucine, lactate, laurate, laurylcarnitine, leucine, maltose, myo-inositol, N-acetylornithine, octanoylcarnitine, phenylalanine, phosphate, proline, propionylcarnitine, pyruvate, serine, stearidonate (18:4n3), threonine, X-07765, X-11302, X-11421, X-11423, X-11793, X-11838, X-12206, X-12405, X-12465, X-12644, X-12794, X-12802, or X-12855.

18. A kit for diagnosing sepsis by identifying a human subject at high risk for sepsis death comprising: a reagent for detecting a clinico-metabolomic classifier, wherein the clinico-metabolomic classifier is 1-arachidonoyl-GPC, 1-methylimidazole acetate, 1-palmitoleoyl-GPC, 2-methoxyacetaminophen sulfate, 3-(4-hydroxyphenyl)lactate (HPLA), 3-methoxytyrosine, 3-methylhistidine, 4-vinylphenol sulfate, 7-alpha-hydroxy-3-oxo-4-cholestenoate (7-HOCA), acetylcarnitine, alanine, androsterone sulfate, biliverdin, butyrylcarnitine, caproate, c-glycosyltryptophan, chenodeoxycholate, creatinine, decanoylcarnitine, dihydroxyacetone, epiandrosterone sulfate, gulono-1,4-lactone, heme, lactate, laurate, laurylcarnitine, N-acetylneuraminate, octanoylcarnitine, palmitoylcarnitine, piperine, piperine, propionylcarnitine, pyruvate, quinate, γ-tocopherol, X-02249, X-03056, X-06126, X-07765, X-10395, X-11255, X-11261, X-11302, X-11421, X-11444, X-11445, X-11450, X-11478, X-11538, X-11546, X-11809, X-11826, X-11843, X-11908, X-12051, X-12095, X-12100, X-12217, X-12405, X-12695, X-12742, X-12755, X-13553, or X-14626.
